SEQUENCE LISTING (255 sequences)

SEQ ID NO: 1 - DNA, 21 bp, Artificial Sequence
  Description of combined DNA/RNA molecule Synthetic oligonucleotide
ggaucuuauuucuucggagtt

SEQ ID NO: 2 - DNA, 21 bp, Artificial Sequence
  Description of combined DNA/RNA molecule Synthetic oligonucleotide
ggaucuuauuucuucggagtt

SEQ ID NO: 3 - DNA, 25 bp, Artificial Sequence
  Description of combined DNA/RNA molecule Synthetic oligonucleotide
agacagcgaccaaaagaauucggat

SEQ ID NO: 4 - DNA, 25 bp, Artificial Sequence
  Description of combined DNA/RNA molecule Synthetic oligonucleotide
agacagcgaccaaaagaauucggat

SEQ ID NO: 5 - DNA, 25 bp, Artificial Sequence
  Description of combined DNA/RNA molecule Synthetic oligonucleotide
augaagaucuguuccaccauugaag

SEQ ID NO: 6 - DNA, 25 bp, Artificial Sequence
  Description of combined DNA/RNA molecule Synthetic oligonucleotide
augaagaucuguuccaccauugaag

SEQ ID NO: 7 - DNA, 25 bp, Artificial Sequence
  Description of combined DNA/RNA molecule Synthetic oligonucleotide
gaucuguuccaccauugaagaactc

SEQ ID NO: 8 - DNA, 25 bp, Artificial Sequence
  Description of combined DNA/RNA molecule Synthetic oligonucleotide
gaucuguuccaccauugaagaactc

SEQ ID NO: 9 - DNA, 25 bp, Artificial Sequence
  Description of combined DNA/RNA molecule Synthetic oligonucleotide
uugaggagugccugauuaaugaucc

SEQ ID NO: 10 - DNA, 25 bp, Artificial Sequence
  Description of combined DNA/RNA molecule Synthetic oligonucleotide
uugaggagugccugauuaaugaucc

SEQ ID NO: 11 - DNA, 21 bp, Artificial Sequence
  Description of combined DNA/RNA molecule Synthetic oligonucleotide
ggcucuuauuucuucggagtt

SEQ ID NO: 12 - DNA, 21 bp, Artificial Sequence
  Description of combined DNA/RNA molecule Synthetic oligonucleotide
ggauccuauuucuucggagtt

SEQ ID NO: 13 - DNA, 21 bp, Artificial Sequence
  Description of combined DNA/RNA molecule Synthetic oligonucleotide
ggaucuuacuucuucggagtt

SEQ ID NO: 14 - DNA, 21 bp, Artificial Sequence
  Description of combined DNA/RNA molecule Synthetic oligonucleotide
ggaucuuauuucuucggagtt

SEQ ID NO: 15 - DNA, 21 bp, Artificial Sequence
  Description of combined DNA/RNA molecule Synthetic oligonucleotide
ggaucuuauuucuucggagtt

SEQ ID NO: 16 - DNA, 21 bp, Artificial Sequence
  Description of combined DNA/RNA molecule Synthetic oligonucleotide
ggaucuuauuucuucggagtt

SEQ ID NO: 17 - DNA, 21 bp, Artificial Sequence
  Description of combined DNA/RNA molecule Synthetic oligonucleotide
ggaucuuauuucuucggagtt

SEQ ID NO: 18 - DNA, 21 bp, Artificial Sequence
  Description of combined DNA/RNA molecule Synthetic oligonucleotide
ggaucuuauuucuucggagtt

SEQ ID NO: 19 - DNA, 21 bp, Artificial Sequence
  Description of combined DNA/RNA molecule Synthetic oligonucleotide
ggaucuuauuucuucggagtt

SEQ ID NO: 20 - DNA, 21 bp, Artificial Sequence
  Description of combined DNA/RNA molecule Synthetic oligonucleotide
ggaucuuauuucuucggagtt

SEQ ID NO: 21 - DNA, 21 bp, Artificial Sequence
  Description of combined DNA/RNA molecule Synthetic oligonucleotide
ggaucuuauuucuucggcgtt

SEQ ID NO: 22 - DNA, 21 bp, Artificial Sequence
  Description of combined DNA/RNA molecule Synthetic oligonucleotide
gaucuuauuucuucggagatt

SEQ ID NO: 23 - DNA, 21 bp, Artificial Sequence
  Description of combined DNA/RNA molecule Synthetic oligonucleotide
gaucuuauuucuucggagatt

SEQ ID NO: 24 - DNA, 21 bp, Artificial Sequence
  Description of combined DNA/RNA molecule Synthetic oligonucleotide
gaucuuauuucuucggagatt

SEQ ID NO: 25 - DNA, 21 bp, Artificial Sequence
  Description of combined DNA/RNA molecule Synthetic oligonucleotide
gaucuuauuucuucggagatt

SEQ ID NO: 26 - DNA, 21 bp, Artificial Sequence
  Description of combined DNA/RNA molecule Synthetic oligonucleotide
gaucuuauuucuucggagatt

SEQ ID NO: 27 - DNA, 21 bp, Artificial Sequence
  Description of combined DNA/RNA molecule Synthetic oligonucleotide
gaucuuauuucuucggagatt

SEQ ID NO: 28 - DNA, 21 bp, Artificial Sequence
  Description of combined DNA/RNA molecule Synthetic oligonucleotide
gaucuuauuucuucggagatt

SEQ ID NO: 29 - DNA, 21 bp, Artificial Sequence
  Description of combined DNA/RNA molecule Synthetic oligonucleotide
gcucuuauuucuucggagatt

SEQ ID NO: 30 - DNA, 21 bp, Artificial Sequence
  Description of combined DNA/RNA molecule Synthetic oligonucleotide
gauccuauuucuucggagatt

SEQ ID NO: 31 - DNA, 21 bp, Artificial Sequence
  Description of combined DNA/RNA molecule Synthetic oligonucleotide
gaucuuacuucuucggagatt

SEQ ID NO: 32 - DNA, 21 bp, Artificial Sequence
  Description of combined DNA/RNA molecule Synthetic oligonucleotide
gaucuuauuucuucggcgatt

SEQ ID NO: 33 - DNA, 24 bp, Artificial Sequence
  Description of combined DNA/RNA molecule Synthetic oligonucleotide
gaucuuauuucuucggagacaatg

SEQ ID NO: 34 - DNA, 23 bp, Artificial Sequence
  Description of combined DNA/RNA molecule Synthetic oligonucleotide
aucuuauuucuucggagacaatg

SEQ ID NO: 35 - DNA, 25 bp, Artificial Sequence
  Description of combined DNA/RNA molecule Synthetic oligonucleotide
ggaucuuauuucuucggagacaatg

SEQ ID NO: 36 - DNA, 25 bp, Artificial Sequence
  Description of combined DNA/RNA molecule Synthetic oligonucleotide
ggaucuuauuucuucggagacaatg

SEQ ID NO: 37 - DNA, 25 bp, Artificial Sequence
  Description of combined DNA/RNA molecule Synthetic oligonucleotide
ggaucuuauuucuucggagacaatg

SEQ ID NO: 38 - DNA, 25 bp, Artificial Sequence
  Description of combined DNA/RNA molecule Synthetic oligonucleotide
ggaucuuauuucuucggagacaatg

SEQ ID NO: 39 - DNA, 25 bp, Artificial Sequence
  Description of combined DNA/RNA molecule Synthetic oligonucleotide
ggaucuuauuucuucggagacaatg

SEQ ID NO: 40 - DNA, 25 bp, Artificial Sequence
  Description of combined DNA/RNA molecule Synthetic oligonucleotide
ggaucuuauuucuucggagacaatg

SEQ ID NO: 41 - DNA, 21 bp, Artificial Sequence
  Description of combined DNA/RNA molecule Synthetic oligonucleotide
gaucuguuccaccauugaatt

SEQ ID NO: 42 - DNA, 21 bp, Artificial Sequence
  Description of combined DNA/RNA molecule Synthetic oligonucleotide
augaagaucuguuccaccatt

SEQ ID NO: 43 - DNA, 21 bp, Artificial Sequence
  Description of combined DNA/RNA molecule Synthetic oligonucleotide
gcaauugaggagugccugatt

SEQ ID NO: 44 - DNA, 21 bp, Artificial Sequence
  Description of combined DNA/RNA molecule Synthetic oligonucleotide
uugaggagugccugauuaatt

SEQ ID NO: 45 - DNA, 21 bp, Artificial Sequence
  Description of combined DNA/RNA molecule Synthetic oligonucleotide
cgggacucuagcauacuuatt

SEQ ID NO: 46 - DNA, 21 bp, Artificial Sequence
  Description of combined DNA/RNA molecule Synthetic oligonucleotide
acugacagccagacagcgatt

SEQ ID NO: 47 - DNA, 21 bp, Artificial Sequence
  Description of combined DNA/RNA molecule Synthetic oligonucleotide
agacagcgaccaaaagaautt

SEQ ID NO: 48 - DNA, 21 bp, Artificial Sequence
  Description of combined DNA/RNA molecule Synthetic oligonucleotide
ggatcttatttcttcggagtt

SEQ ID NO: 49 - DNA, 25 bp, Artificial Sequence
  Description of combined DNA/RNA molecule Synthetic oligonucleotide
ggaucuuauuucuucggagacaatg

SEQ ID NO: 50 - DNA, 25 bp, Artificial Sequence
  Description of combined DNA/RNA molecule Synthetic oligonucleotide
ggaucuuauuucuucggagacaatg

SEQ ID NO: 51 - DNA, 25 bp, Artificial Sequence
  Description of combined DNA/RNA molecule Synthetic oligonucleotide
ggaucuuauuucuucggagacaatt

SEQ ID NO: 52 - DNA, 25 bp, Artificial Sequence
  Description of combined DNA/RNA molecule Synthetic oligonucleotide
ggatcttatttcttcggagacaatg

SEQ ID NO: 53 - DNA, 25 bp, Artificial Sequence
  Description of combined DNA/RNA molecule Synthetic oligonucleotide
ggaucuuauuucuucggagacaatg

SEQ ID NO: 54 - DNA, 25 bp, Artificial Sequence
  Description of combined DNA/RNA molecule Synthetic oligonucleotide
ggaucuuauuucuucggagacaatg

SEQ ID NO: 55 - DNA, 25 bp, Artificial Sequence
  Description of combined DNA/RNA molecule Synthetic oligonucleotide
ggaucuuauuucuucggagacaaug

SEQ ID NO: 56 - DNA, 25 bp, Artificial Sequence
  Description of combined DNA/RNA molecule Synthetic oligonucleotide
ggaucuuauuucuucggagacaatg

SEQ ID NO: 57 - DNA, 25 bp, Artificial Sequence
  Description of combined DNA/RNA molecule Synthetic oligonucleotide
ggatcttatttcttcggagacaatg

SEQ ID NO: 58 - DNA, 25 bp, Artificial Sequence
  Description of combined DNA/RNA molecule Synthetic oligonucleotide
ggatcttatttcttcggagacaatg

SEQ ID NO: 59 - DNA, 21 bp, Artificial Sequence
  Description of combined DNA/RNA molecule Synthetic oligonucleotide
cuccgaagaaauaagaccutt

SEQ ID NO: 60 - DNA, 21 bp, Artificial Sequence
  Description of combined DNA/RNA molecule Synthetic oligonucleotide
cuccgaagaaauaagaucctt

SEQ ID NO: 61 - DNA, 27 bp, Artificial Sequence
  Description of combined DNA/RNA molecule Synthetic oligonucleotide
auccgaauucuuuuggucgcugucutt

SEQ ID NO: 62 - DNA, 27 bp, Artificial Sequence
  Description of combined DNA/RNA molecule Synthetic oligonucleotide
auccgaauucuuuuggucgcugucuuu

SEQ ID NO: 63 - DNA, 27 bp, Artificial Sequence
  Description of combined DNA/RNA molecule Synthetic oligonucleotide
cuucaaugguggaacagaucuucautt

SEQ ID NO: 64 - DNA, 27 bp, Artificial Sequence
  Description of combined DNA/RNA molecule Synthetic oligonucleotide
cuucaaugguggaacagaucuucauuu

SEQ ID NO: 65 - DNA, 27 bp, Artificial Sequence
  Description of combined DNA/RNA molecule Synthetic oligonucleotide
gaguucuucaaugguggaacagauctt

SEQ ID NO: 66 - DNA, 27 bp, Artificial Sequence
  Description of combined DNA/RNA molecule Synthetic oligonucleotide
gaguucuucaaugguggaacagaucuu

SEQ ID NO: 67 - DNA, 27 bp, Artificial Sequence
  Description of combined DNA/RNA molecule Synthetic oligonucleotide
ggaucauuaaucaggcacuccucaatt

SEQ ID NO: 68 - DNA, 27 bp, Artificial Sequence
  Description of combined DNA/RNA molecule Synthetic oligonucleotide
ggaucauuaaucaggcacuccucaauu

SEQ ID NO: 69 - DNA, 21 bp, Artificial Sequence
  Description of combined DNA/RNA molecule Synthetic oligonucleotide
cuccgaagaaauaagancctt

SEQ ID NO: 70 - DNA, 21 bp, Artificial Sequence
  Description of combined DNA/RNA molecule Synthetic oligonucleotide
cuccgaagaaauangaucctt

SEQ ID NO: 71 - DNA, 21 bp, Artificial Sequence
  Description of combined DNA/RNA molecule Synthetic oligonucleotide
cuccgaagaanuaagaucctt

SEQ ID NO: 72 - DNA, 21 bp, Artificial Sequence
  Description of combined DNA/RNA molecule Synthetic oligonucleotide
cuccgaagaaauaagaucctt

SEQ ID NO: 73 - DNA, 21 bp, Artificial Sequence
  Description of combined DNA/RNA molecule Synthetic oligonucleotide
cuccgaanaaauaagaucctt

SEQ ID NO: 74 - DNA, 21 bp, Artificial Sequence
  Description of combined DNA/RNA molecule Synthetic oligonucleotide
cuccnaagaaauaagaucctt

SEQ ID NO: 75 - DNA, 21 bp, Artificial Sequence
  Description of combined DNA/RNA molecule Synthetic oligonucleotide
cuccgaagaaauaagancctt

SEQ ID NO: 76 - DNA, 21 bp, Artificial Sequence
  Description of combined DNA/RNA molecule Synthetic oligonucleotide
cuccgaagaaauangaucctt

SEQ ID NO: 77 - DNA, 21 bp, Artificial Sequence
  Description of combined DNA/RNA molecule Synthetic oligonucleotide
cuccgaagaanuaagaucctt

SEQ ID NO: 78 - DNA, 21 bp, Artificial Sequence
  Description of combined DNA/RNA molecule Synthetic oligonucleotide
cnccgaagaaauaagaucctt

SEQ ID NO: 79 - DNA, 21 bp, Artificial Sequence
  Description of combined DNA/RNA molecule Synthetic oligonucleotide
cnccgaagaaauaagaucctt

SEQ ID NO: 80 - DNA, 21 bp, Artificial Sequence
  Description of combined DNA/RNA molecule Synthetic oligonucleotide
ucuccgaagaaauaagauctt

SEQ ID NO: 81 - DNA, 21 bp, Artificial Sequence
  Description of combined DNA/RNA molecule Synthetic oligonucleotide
ucuccgaanaaauaagauctt

SEQ ID NO: 82 - DNA, 21 bp, Artificial Sequence
  Description of combined DNA/RNA molecule Synthetic oligonucleotide
ucuccnaagaaauaagauctt

SEQ ID NO: 83 - DNA, 21 bp, Artificial Sequence
  Description of combined DNA/RNA molecule Synthetic oligonucleotide
ucuccgaagaaauaaganctt

SEQ ID NO: 84 - DNA, 21 bp, Artificial Sequence
  Description of combined DNA/RNA molecule Synthetic oligonucleotide
ucuccgaagaaauangauctt

SEQ ID NO: 85 - DNA, 21 bp, Artificial Sequence
  Description of combined DNA/RNA molecule Synthetic oligonucleotide
ucuccgaagaanuaagauctt

SEQ ID NO: 86 - DNA, 21 bp, Artificial Sequence
  Description of combined DNA/RNA molecule Synthetic oligonucleotide
ucnccgaagaaauaagauctt

SEQ ID NO: 87 - DNA, 21 bp, Artificial Sequence
  Description of combined DNA/RNA molecule Synthetic oligonucleotide
ucuccgaagaaauaaganctt

SEQ ID NO: 88 - DNA, 21 bp, Artificial Sequence
  Description of combined DNA/RNA molecule Synthetic oligonucleotide
ucuccgaagaaauangauctt

SEQ ID NO: 89 - DNA, 21 bp, Artificial Sequence
  Description of combined DNA/RNA molecule Synthetic oligonucleotide
ucuccgaagaanuaagauctt

SEQ ID NO: 90 - DNA, 21 bp, Artificial Sequence
  Description of combined DNA/RNA molecule Synthetic oligonucleotide
ucnccgaagaaauaagauctt

SEQ ID NO: 91 - DNA, 27 bp, Artificial Sequence
  Description of combined DNA/RNA molecule Synthetic oligonucleotide
cauugucuccgaagaaauaagauccuu

SEQ ID NO: 92 - DNA, 27 bp, Artificial Sequence
  Description of combined DNA/RNA molecule Synthetic oligonucleotide
cauugucuccgaagaaauaagauccuu

SEQ ID NO: 93 - DNA, 27 bp, Artificial Sequence
  Description of combined DNA/RNA molecule Synthetic oligonucleotide
cauugucuccgaagaaauaagaucctt

SEQ ID NO: 94 - DNA, 27 bp, Artificial Sequence
  Description of combined DNA/RNA molecule Synthetic oligonucleotide
cauugucuccgaagaaauaagauccuu

SEQ ID NO: 95 - DNA, 27 bp, Artificial Sequence
  Description of combined DNA/RNA molecule Synthetic oligonucleotide
cauugucuccgaagaaauaagaucctt

SEQ ID NO: 96 - DNA, 26 bp, Artificial Sequence
  Description of combined DNA/RNA molecule Synthetic oligonucleotide
auugucuccgaagaaauaagauccuu

SEQ ID NO: 97 - DNA, 25 bp, Artificial Sequence
  Description of combined DNA/RNA molecule Synthetic oligonucleotide
uugucuccgaagaaauaagauccuu

SEQ ID NO: 98 - DNA, 27 bp, Artificial Sequence
  Description of combined DNA/RNA molecule Synthetic oligonucleotide
cauugucuccgaagaaauaagauccuu

SEQ ID NO: 99 - DNA, 21 bp, Artificial Sequence
  Description of combined DNA/RNA molecule Synthetic oligonucleotide
uucaaugguggaacagauctt

SEQ ID NO: 100 - DNA, 21 bp, Artificial Sequence
  Description of combined DNA/RNA molecule Synthetic oligonucleotide
ugguggaacagaucuucautt

SEQ ID NO: 101 - DNA, 21 bp, Artificial Sequence
  Description of combined DNA/RNA molecule Synthetic oligonucleotide
ucaggcacuccucaauugctt

SEQ ID NO: 102 - DNA, 21 bp, Artificial Sequence
  Description of combined DNA/RNA molecule Synthetic oligonucleotide
uuaaucaggcacuccucaatt

SEQ ID NO: 103 - DNA, 21 bp, Artificial Sequence
  Description of combined DNA/RNA molecule Synthetic oligonucleotide
uaaguaugcuagagucccgtt

SEQ ID NO: 104 - DNA, 21 bp, Artificial Sequence
  Description of combined DNA/RNA molecule Synthetic oligonucleotide
ucgcugucuggcugucagutt

SEQ ID NO: 105 - DNA, 21 bp, Artificial Sequence
  Description of combined DNA/RNA molecule Synthetic oligonucleotide
auucuuuuggucgcugucutt

SEQ ID NO: 106 - DNA, 21 bp, Artificial Sequence
  Description of combined DNA/RNA molecule Synthetic oligonucleotide
ctccgaagaaataagatcctt

SEQ ID NO: 107 - DNA, 27 bp, Artificial Sequence
  Description of combined DNA/RNA molecule Synthetic oligonucleotide
cauugucuccgaagaaauaagauccuu

SEQ ID NO: 108 - DNA, 27 bp, Artificial Sequence
  Description of combined DNA/RNA molecule Synthetic oligonucleotide
cauugucuccgaagaaauaagauccuu

SEQ ID NO: 109 - DNA, 27 bp, Artificial Sequence
  Description of combined DNA/RNA molecule Synthetic oligonucleotide
aauugucuccgaagaaauaagauccuu

SEQ ID NO: 110 - DNA, 27 bp, Artificial Sequence
  Description of combined DNA/RNA molecule Synthetic oligonucleotide
cattgtctccgaagaaataagatccuu

SEQ ID NO: 111 - DNA, 27 bp, Artificial Sequence
  Description of combined DNA/RNA molecule Synthetic oligonucleotide
cauugucuccgaagaaauaagauccuu

SEQ ID NO: 112 - DNA, 27 bp, Artificial Sequence
  Description of combined DNA/RNA molecule Synthetic oligonucleotide
cauugucuccgaagaaauaagauccuu

SEQ ID NO: 113 - DNA, 27 bp, Artificial Sequence
  Description of combined DNA/RNA molecule Synthetic oligonucleotide
cauugucuccgaagaaauaagauccuu

SEQ ID NO: 114 - DNA, 27 bp, Artificial Sequence
  Description of combined DNA/RNA molecule Synthetic oligonucleotide
cauugucuccgaagaaauaagauccuu

SEQ ID NO: 115 - DNA, 27 bp, Artificial Sequence
  Description of combined DNA/RNA molecule Synthetic oligonucleotide
cattgtctccgaagaaataagatcctt

SEQ ID NO: 116 - DNA, 27 bp, Artificial Sequence
  Description of combined DNA/RNA molecule Synthetic oligonucleotide
cattgtctccgaagaaataagatccuu

SEQ ID NO: 117 - RNA, 19 nt, Artificial Sequence
  Description of Artificial Sequence Synthetic oligonucleotide
agacagcgaccaaaagaau

SEQ ID NO: 118 - RNA, 19 nt, Artificial Sequence
  Description of Artificial Sequence Synthetic oligonucleotide
agacagcgaccaaaaggau

SEQ ID NO: 119 - RNA, 19 nt, Artificial Sequence
  Description of Artificial Sequence Synthetic oligonucleotide
agacagcgaccaaaggaau

SEQ ID NO: 120 - RNA, 19 nt, Artificial Sequence
  Description of Artificial Sequence Synthetic oligonucleotide
agacagcgaccaaaagauu

SEQ ID NO: 121 - RNA, 19 nt, Artificial Sequence
  Description of Artificial Sequence Synthetic oligonucleotide
agacgacgaucaaaagaau

SEQ ID NO: 122 - RNA, 19 nt, Artificial Sequence
  Description of Artificial Sequence Synthetic oligonucleotide
acugacagccagacagcga

SEQ ID NO: 123 - RNA, 19 nt, Artificial Sequence
  Description of Artificial Sequence Synthetic oligonucleotide
acugacagucagacagcga

SEQ ID NO: 124 - RNA, 19 nt, Artificial Sequence
  Description of Artificial Sequence Synthetic oligonucleotide
acugauagccagacagcga

SEQ ID NO: 125 - RNA, 19 nt, Artificial Sequence
  Description of Artificial Sequence Synthetic oligonucleotide
accgacagccagacagcga

SEQ ID NO: 126 - RNA, 19 nt, Artificial Sequence
  Description of Artificial Sequence Synthetic oligonucleotide
acugacagccagacgacga

SEQ ID NO: 127 - RNA, 19 nt, Artificial Sequence
  Description of Artificial Sequence Synthetic oligonucleotide
cgggacucuagcauacuua

SEQ ID NO: 128 - RNA, 19 nt, Artificial Sequence
  Description of Artificial Sequence Synthetic oligonucleotide
cgggacucuagcaugcuua

SEQ ID NO: 129 - RNA, 19 nt, Artificial Sequence
  Description of Artificial Sequence Synthetic oligonucleotide
cgggacuuuagcauacuua

SEQ ID NO: 130 - RNA, 19 nt, Artificial Sequence
  Description of Artificial Sequence Synthetic oligonucleotide
agggacucuagcauacuua

SEQ ID NO: 131 - RNA, 19 nt, Artificial Sequence
  Description of Artificial Sequence Synthetic oligonucleotide
cgagacucuagcauacuua

SEQ ID NO: 132 - RNA, 19 nt, Artificial Sequence
  Description of Artificial Sequence Synthetic oligonucleotide
cggaacucuagcauacuua

SEQ ID NO: 133 - RNA, 19 nt, Artificial Sequence
  Description of Artificial Sequence Synthetic oligonucleotide
cgggacuauagcauacuua

SEQ ID NO: 134 - RNA, 19 nt, Artificial Sequence
  Description of Artificial Sequence Synthetic oligonucleotide
cgggacuccagcauacuua

SEQ ID NO: 135 - RNA, 19 nt, Artificial Sequence
  Description of Artificial Sequence Synthetic oligonucleotide
cgggacucuaacauacuua

SEQ ID NO: 136 - RNA, 19 nt, Artificial Sequence
  Description of Artificial Sequence Synthetic oligonucleotide
gaucuguuccaccauugaa

SEQ ID NO: 137 - RNA, 19 nt, Artificial Sequence
  Description of Artificial Sequence Synthetic oligonucleotide
gaucuguuucaccauugaa

SEQ ID NO: 138 - RNA, 19 nt, Artificial Sequence
  Description of Artificial Sequence Synthetic oligonucleotide
aaucuguuccaccauugaa

SEQ ID NO: 139 - RNA, 19 nt, Artificial Sequence
  Description of Artificial Sequence Synthetic oligonucleotide
gaccuguuccaccauugaa

SEQ ID NO: 140 - RNA, 19 nt, Artificial Sequence
  Description of Artificial Sequence Synthetic oligonucleotide
gaucugcuccaccauugaa

SEQ ID NO: 141 - RNA, 19 nt, Artificial Sequence
  Description of Artificial Sequence Synthetic oligonucleotide
gaucuguuacaccauugaa

SEQ ID NO: 142 - RNA, 19 nt, Artificial Sequence
  Description of Artificial Sequence Synthetic oligonucleotide
gaucuguuccaccauuaaa

SEQ ID NO: 143 - RNA, 19 nt, Artificial Sequence
  Description of Artificial Sequence Synthetic oligonucleotide
gaccuguucuaccauugaa

SEQ ID NO: 144 - RNA, 19 nt, Artificial Sequence
  Description of Artificial Sequence Synthetic oligonucleotide
gaccugcuccaccauugaa

SEQ ID NO: 145 - RNA, 19 nt, Artificial Sequence
  Description of Artificial Sequence Synthetic oligonucleotide
augaagaucuguuccacca

SEQ ID NO: 146 - RNA, 19 nt, Artificial Sequence
  Description of Artificial Sequence Synthetic oligonucleotide
augaagaucuguuucacca

SEQ ID NO: 147 - RNA, 19 nt, Artificial Sequence
  Description of Artificial Sequence Synthetic oligonucleotide
augaggaucuguuccacca

SEQ ID NO: 148 - RNA, 19 nt, Artificial Sequence
  Description of Artificial Sequence Synthetic oligonucleotide
acgaagaucuguuccacca

SEQ ID NO: 149 - RNA, 19 nt, Artificial Sequence
  Description of Artificial Sequence Synthetic oligonucleotide
augaaaaucuguuccacca

SEQ ID NO: 150 - RNA, 19 nt, Artificial Sequence
  Description of Artificial Sequence Synthetic oligonucleotide
augaagaccuguuccacca

SEQ ID NO: 151 - RNA, 19 nt, Artificial Sequence
  Description of Artificial Sequence Synthetic oligonucleotide
augaagaucugcuccacca

SEQ ID NO: 152 - RNA, 19 nt, Artificial Sequence
  Description of Artificial Sequence Synthetic oligonucleotide
augaagaucuguuacacca

SEQ ID NO: 153 - RNA, 19 nt, Artificial Sequence
  Description of Artificial Sequence Synthetic oligonucleotide
cugaagaucuguuccacca

SEQ ID NO: 154 - RNA, 19 nt, Artificial Sequence
  Description of Artificial Sequence Synthetic oligonucleotide
uugaagaucuguuccacca

SEQ ID NO: 155 - RNA, 19 nt, Artificial Sequence
  Description of Artificial Sequence Synthetic oligonucleotide
augaagaccuguucuacca

SEQ ID NO: 156 - RNA, 19 nt, Artificial Sequence
  Description of Artificial Sequence Synthetic oligonucleotide
auaaagaccuguuccacca

SEQ ID NO: 157 - RNA, 19 nt, Artificial Sequence
  Description of Artificial Sequence Synthetic oligonucleotide
augaagaccugcuccacca

SEQ ID NO: 158 - RNA, 19 nt, Artificial Sequence
  Description of Artificial Sequence Synthetic oligonucleotide
uugaggagugccugauuaa

SEQ ID NO: 159 - RNA, 19 nt, Artificial Sequence
  Description of Artificial Sequence Synthetic oligonucleotide
uugaggagugccugguuaa

SEQ ID NO: 160 - RNA, 19 nt, Artificial Sequence
  Description of Artificial Sequence Synthetic oligonucleotide
uugaggaaugccugauuaa

SEQ ID NO: 161 - RNA, 19 nt, Artificial Sequence
  Description of Artificial Sequence Synthetic oligonucleotide
uugaggagugccuaauuaa

SEQ ID NO: 162 - RNA, 19 nt, Artificial Sequence
  Description of Artificial Sequence Synthetic oligonucleotide
gcaauugaggagugccuga

SEQ ID NO: 163 - RNA, 19 nt, Artificial Sequence
  Description of Artificial Sequence Synthetic oligonucleotide
gcaauugaggagugccugg

SEQ ID NO: 164 - RNA, 19 nt, Artificial Sequence
  Description of Artificial Sequence Synthetic oligonucleotide
gcaguugaggagugccuga

SEQ ID NO: 165 - RNA, 19 nt, Artificial Sequence
  Description of Artificial Sequence Synthetic oligonucleotide
gcaauugaggaaugccuga

SEQ ID NO: 166 - RNA, 19 nt, Artificial Sequence
  Description of Artificial Sequence Synthetic oligonucleotide
gcaauugaggagugccuaa

SEQ ID NO: 167 - RNA, 19 nt, Artificial Sequence
  Description of Artificial Sequence Synthetic oligonucleotide
gaucuuauuucuucggaga

SEQ ID NO: 168 - RNA, 19 nt, Artificial Sequence
  Description of Artificial Sequence Synthetic oligonucleotide
gaucuuauuucuucgggga

SEQ ID NO: 169 - RNA, 19 nt, Artificial Sequence
  Description of Artificial Sequence Synthetic oligonucleotide
gaucuuauuucuuuggaga

SEQ ID NO: 170 - RNA, 19 nt, Artificial Sequence
  Description of Artificial Sequence Synthetic oligonucleotide
ggucuuauuucuucggaga

SEQ ID NO: 171 - RNA, 19 nt, Artificial Sequence
  Description of Artificial Sequence Synthetic oligonucleotide
guucuuauuucuucggaga

SEQ ID NO: 172 - RNA, 19 nt, Artificial Sequence
  Description of Artificial Sequence Synthetic oligonucleotide
ggaucuuauuucuucggag

SEQ ID NO: 173 - RNA, 19 nt, Artificial Sequence
  Description of Artificial Sequence Synthetic oligonucleotide
ggaucuuauuucuucgggg

SEQ ID NO: 174 - RNA, 19 nt, Artificial Sequence
  Description of Artificial Sequence Synthetic oligonucleotide
ggaucuuauuucuuuggag

SEQ ID NO: 175 - RNA, 19 nt, Artificial Sequence
  Description of Artificial Sequence Synthetic oligonucleotide
gggucuuauuucuucggag

SEQ ID NO: 176 - RNA, 19 nt, Artificial Sequence
  Description of Artificial Sequence Synthetic oligonucleotide
gguucuuauuucuucggag

SEQ ID NO: 177 - DNA, 21 bp, Artificial Sequence
  Description of combined DNA/RNA molecule Synthetic oligonucleotide
ggagacgugguguugguaatt

SEQ ID NO: 178 - DNA, 21 bp, Artificial Sequence
  Description of combined DNA/RNA molecule Synthetic oligonucleotide
uuaccaacaccacgucucctt

SEQ ID NO: 179 - DNA, 21 bp, Artificial Sequence
  Description of combined DNA/RNA molecule Synthetic oligonucleotide
cgggacucuagcauacuuatt

SEQ ID NO: 180 - DNA, 21 bp, Artificial Sequence
  Description of combined DNA/RNA molecule Synthetic oligonucleotide
uaaguaugcuagagucccgtt

SEQ ID NO: 181 - DNA, 21 bp, Artificial Sequence
  Description of combined DNA/RNA molecule Synthetic oligonucleotide
gcaggcaaaccauuugaautt

SEQ ID NO: 182 - DNA, 21 bp, Artificial Sequence
  Description of combined DNA/RNA molecule Synthetic oligonucleotide
auucaaaugguuugccugctt

SEQ ID NO: 183 - DNA, 21 bp, Artificial Sequence
  Description of combined DNA/RNA molecule Synthetic oligonucleotide
caggauacaccauggauactt

SEQ ID NO: 184 - DNA, 21 bp, Artificial Sequence
  Description of combined DNA/RNA molecule Synthetic oligonucleotide
guauccaugguguauccugtt

SEQ ID NO: 185 - DNA, 21 bp, Artificial Sequence
  Description of combined DNA/RNA molecule Synthetic oligonucleotide
gaucuguuccaccauugaatt

SEQ ID NO: 186 - DNA, 21 bp, Artificial Sequence
  Description of combined DNA/RNA molecule Synthetic oligonucleotide
uucaaugguggaacagauctt

SEQ ID NO: 187 - DNA, 21 bp, Artificial Sequence
  Description of combined DNA/RNA molecule Synthetic oligonucleotide
ugcuucaauccgaugauugtt

SEQ ID NO: 188 - DNA, 21 bp, Artificial Sequence
  Description of combined DNA/RNA molecule Synthetic oligonucleotide
caaucaucggauugaagcatt

SEQ ID NO: 189 - DNA, 21 bp, Artificial Sequence
  Description of combined DNA/RNA molecule Synthetic oligonucleotide
cggcuacauugagggcaagtt

SEQ ID NO: 190 - DNA, 21 bp, Artificial Sequence
  Description of combined DNA/RNA molecule Synthetic oligonucleotide
cuugcccucaauguagccgtt

SEQ ID NO: 191 - DNA, 21 bp, Artificial Sequence
  Description of combined DNA/RNA molecule Synthetic oligonucleotide
gcaauugaggagugccugatt

SEQ ID NO: 192 - DNA, 21 bp, Artificial Sequence
  Description of combined DNA/RNA molecule Synthetic oligonucleotide
ucaggcacuccucaauugctt

SEQ ID NO: 193 - DNA, 21 bp, Artificial Sequence
  Description of combined DNA/RNA molecule Synthetic oligonucleotide
ugaucccuggguuuugcuutt

SEQ ID NO: 194 - DNA, 21 bp, Artificial Sequence
  Description of combined DNA/RNA molecule Synthetic oligonucleotide
aagcaaaacccagggaucatt

SEQ ID NO: 195 - DNA, 21 bp, Artificial Sequence
  Description of combined DNA/RNA molecule Synthetic oligonucleotide
ugcuucuugguucaacucctt

SEQ ID NO: 196 - DNA, 21 bp, Artificial Sequence
  Description of combined DNA/RNA molecule Synthetic oligonucleotide
ggaguugaaccaagaagcatt

SEQ ID NO: 197 - DNA, 21 bp, Artificial Sequence
  Description of combined DNA/RNA molecule Synthetic oligonucleotide
uagagagaauggugcucuctt

SEQ ID NO: 198 - DNA, 21 bp, Artificial Sequence
  Description of combined DNA/RNA molecule Synthetic oligonucleotide
gagagcaccauucucucuatt

SEQ ID NO: 199 - DNA, 21 bp, Artificial Sequence
  Description of combined DNA/RNA molecule Synthetic oligonucleotide
uaaggcgaaucuggcgccatt

SEQ ID NO: 200 - DNA, 21 bp, Artificial Sequence
  Description of combined DNA/RNA molecule Synthetic oligonucleotide
uggcgccagauucgccuuatt

SEQ ID NO: 201 - DNA, 21 bp, Artificial Sequence
  Description of combined DNA/RNA molecule Synthetic oligonucleotide
ggaucuuauuucuucggagtt

SEQ ID NO: 202 - DNA, 21 bp, Artificial Sequence
  Description of combined DNA/RNA molecule Synthetic oligonucleotide
cuccgaagaaauaagaucctt

SEQ ID NO: 203 - DNA, 22 bp, Artificial Sequence
  Description of combined DNA/RNA molecule Synthetic oligonucleotide
ggaucuuauuucuucggagatt

SEQ ID NO: 204 - DNA, 22 bp, Artificial Sequence
  Description of combined DNA/RNA molecule Synthetic oligonucleotide
ucuccgaagaaauaagaucctt

SEQ ID NO: 205 - DNA, 21 bp, Artificial Sequence
  Description of combined DNA/RNA molecule Synthetic oligonucleotide
ccgaggucgaaacguacgutt

SEQ ID NO: 206 - DNA, 21 bp, Artificial Sequence
  Description of combined DNA/RNA molecule Synthetic oligonucleotide
acguacguuucgaccucggtt

SEQ ID NO: 207 - DNA, 21 bp, Artificial Sequence
  Description of combined DNA/RNA molecule Synthetic oligonucleotide
cagauugcugacucccagctt

SEQ ID NO: 208 - DNA, 21 bp, Artificial Sequence
  Description of combined DNA/RNA molecule Synthetic oligonucleotide
gcugggagucagcaaucugtt

SEQ ID NO: 209 - DNA, 21 bp, Artificial Sequence
  Description of combined DNA/RNA molecule Synthetic oligonucleotide
uggcuggaucgagugagcatt

SEQ ID NO: 210 - DNA, 21 bp, Artificial Sequence
  Description of combined DNA/RNA molecule Synthetic oligonucleotide
ugcucacucgauccagccatt

SEQ ID NO: 211 - DNA, 21 bp, Artificial Sequence
  Description of combined DNA/RNA molecule Synthetic oligonucleotide
gaauaucgaaaggaacagctt

SEQ ID NO: 212 - DNA, 21 bp, Artificial Sequence
  Description of combined DNA/RNA molecule Synthetic oligonucleotide
gcuguuccuuucgauauuctt

SEQ ID NO: 213 - DNA, 21 bp, Artificial Sequence
  Description of combined DNA/RNA molecule Synthetic oligonucleotide
cggcuucgccgagaucagaat

SEQ ID NO: 214 - DNA, 21 bp, Artificial Sequence
  Description of combined DNA/RNA molecule Synthetic oligonucleotide
ucugaucucggcgaagccgat

SEQ ID NO: 215 - DNA, 21 bp, Artificial Sequence
  Description of combined DNA/RNA molecule Synthetic oligonucleotide
guccuccgaugaggacucctt

SEQ ID NO: 216 - DNA, 21 bp, Artificial Sequence
  Description of combined DNA/RNA molecule Synthetic oligonucleotide
ggaguccucaucggaggactt

SEQ ID NO: 217 - DNA, 21 bp, Artificial Sequence
  Description of combined DNA/RNA molecule Synthetic oligonucleotide
ugauaacacaguucgaguctt

SEQ ID NO: 218 - DNA, 21 bp, Artificial Sequence
  Description of combined DNA/RNA molecule Synthetic oligonucleotide
gacucgaacuguguuaucatt

SEQ ID NO: 219 - RNA, 21 nt, Artificial Sequence
  Description of Artificial Sequence Synthetic oligonucleotide
ggcuacguccaggagcgcauu

SEQ ID NO: 220 - RNA, 21 nt, Artificial Sequence
  Description of Artificial Sequence Synthetic oligonucleotide
ugcgcuccuggacguagccuu

SEQ ID NO: 221 - DNA, 18 bp, Artificial Sequence
  Description of Artificial Sequence Synthetic primer
tttttttttttttttttt

SEQ ID NO: 222 - DNA, 22 bp, Artificial Sequence
  Description of Artificial Sequence Synthetic primer
ctcgtcgcttatgacaaagaag

SEQ ID NO: 223 - DNA, 36 bp, Artificial Sequence
  Description of Artificial Sequence Synthetic primer
atatcgtctcgtattagtagaaacaagggtattttt

SEQ ID NO: 224 - DNA, 21 bp, Artificial Sequence
  Description of Artificial Sequence Synthetic primer
caggacatactgatgaggatg

SEQ ID NO: 225 - DNA, 35 bp, Artificial Sequence
  Description of Artificial Sequence Synthetic primer
atatcgtctcgtattagtagaaacaagggtgtttt

SEQ ID NO: 226 - DNA, 22 bp, Artificial Sequence
  Description of Artificial Sequence Synthetic primer
ctcgtcgcttatgacaaagaag

SEQ ID NO: 227 - DNA, 21 bp, Artificial Sequence
  Description of Artificial Sequence Synthetic primer
agatcatcatgtgagtcagac

SEQ ID NO: 228 - DNA, 21 bp, Artificial Sequence
  Description of Artificial Sequence Synthetic primer
caggacatactgatgaggatg

SEQ ID NO: 229 - DNA, 21 bp, Artificial Sequence
  Description of Artificial Sequence Synthetic primer
gtttcagagactcgaactgtg

SEQ ID NO: 230 - RNA, 19 nt, Artificial Sequence
  Description of Artificial Sequence Synthetic oligonucleotide
agcaaaagcaggguagaua

SEQ ID NO: 231 - RNA, 19 nt, Artificial Sequence
  Description of Artificial Sequence Synthetic oligonucleotide
gcaaaagcaggguagauaa

SEQ ID NO: 232 - RNA, 19 nt, Artificial Sequence
  Description of Artificial Sequence Synthetic oligonucleotide
caaaagcaggguagauaau

SEQ ID NO: 233 - RNA, 19 nt, Artificial Sequence
  Description of Artificial Sequence Synthetic oligonucleotide
aaaagcaggguagauaauc

SEQ ID NO: 234 - RNA, 19 nt, Artificial Sequence
  Description of Artificial Sequence Synthetic oligonucleotide
aaagcaggguagauaauca

SEQ ID NO: 235 - RNA, 19 nt, Artificial Sequence
  Description of Artificial Sequence Synthetic oligonucleotide
aagcaggguagauaaucac

SEQ ID NO: 236 - DNA, 25 bp, Artificial Sequence
  Description of combined DNA/RNA molecule Synthetic oligonucleotide
ggaucuuauuucuucggagacaatg

SEQ ID NO: 237 - RNA, 27 nt, Artificial Sequence
  Description of Artificial Sequence Synthetic oligonucleotide
cauugucuccgaagaaauaagauccuu

SEQ ID NO: 238 - DNA, 21 bp, Artificial Sequence
  Description of combined DNA/RNA molecule Synthetic oligonucleotide
uucuccgaacgugucacgutt

SEQ ID NO: 239 - DNA, 21 bp, Artificial Sequence
  Description of combined DNA/RNA molecule Synthetic oligonucleotide
acgugacacguucggagaatt

SEQ ID NO: 240 - DNA, 21 bp, Artificial Sequence
  Description of combined DNA/RNA molecule Synthetic oligonucleotide
cuacacaaaucagcgauuutt

SEQ ID NO: 241 - DNA, 21 bp, Artificial Sequence
  Description of combined DNA/RNA molecule Synthetic oligonucleotide
aaaucgcugauuuguguagtc

SEQ ID NO: 242 - RNA, 21 nt, Artificial Sequence
  Description of Artificial Sequence Synthetic oligonucleotide
ggaaagacuguuccaaaaauu

SEQ ID NO: 243 - RNA, 21 nt, Artificial Sequence
  Description of Artificial Sequence Synthetic oligonucleotide
uuuuuggaacagucuuuccuu

SEQ ID NO: 244 - DNA, 21 bp, Artificial Sequence
  Description of combined DNA/RNA molecule Synthetic oligonucleotide
ggaucuuauuucuucggagtt

SEQ ID NO: 245 - DNA, 21 bp, Artificial Sequence
  Description of combined DNA/RNA molecule Synthetic oligonucleotide
cuccgaagaaauaagaucctt

SEQ ID NO: 246 - DNA, 25 bp, Artificial Sequence
  Description of combined DNA/RNA molecule Synthetic oligonucleotide
agacagcgaccaaaagaauucggat

SEQ ID NO: 247 - RNA, 27 nt, Artificial Sequence
  Description of Artificial Sequence Synthetic oligonucleotide
auccgaauucuuuuggucgcugucuuu

SEQ ID NO: 248 - DNA, 25 bp, Artificial Sequence
  Description of combined DNA/RNA molecule Synthetic oligonucleotide
cgggactctagcatacttactgaca

SEQ ID NO: 249 - DNA, 27 bp, Artificial Sequence
  Description of combined DNA/RNA molecule Synthetic oligonucleotide
tgtcagtaagtatgctagagtcccguu

SEQ ID NO: 250 - DNA, 25 bp, Artificial Sequence
  Description of combined DNA/RNA molecule Synthetic oligonucleotide
gaucuguuccaccauugaagaactc

SEQ ID NO: 251 - RNA, 27 nt, Artificial Sequence
  Description of Artificial Sequence Synthetic oligonucleotide
gaguucuucaaugguggaacagaucuu

SEQ ID NO: 252 - DNA, 25 bp, Artificial Sequence
  Description of combined DNA/RNA molecule Synthetic oligonucleotide
uugaggagugccugauuaaugaucc

SEQ ID NO: 253 - RNA, 27 nt, Artificial Sequence
  Description of Artificial Sequence Synthetic oligonucleotide
ggaucauuaaucaggcacuccucaauu

SEQ ID NO: 254 - DNA, 25 bp, Artificial Sequence
  Description of combined DNA/RNA molecule Synthetic oligonucleotide
augaagaucuguuccaccauugaag

SEQ ID NO: 255 - RNA, 27 nt, Artificial Sequence
  Description of Artificial Sequence Synthetic oligonucleotide
cuucaaugguggaacagaucuucauuu

(19) United States
(12) Patent Application Publication
(10) Pub. No.: US 2009/0042823 A1
(43) Pub. Date: Feb. 12, 2009

(54) USES OF BROAD SPECTRUM RNAI THERAPEUTICS AGAINST INFLUENZA

(75) Inventors: Michael V. Templin, Bothell, WA (US); Shaguna Seth, Bothell, WA (US); Gregory Mark Severson, Lynnwood, WA (US)

Correspondence Address: NASTECH PHARMACEUTICAL COMPANY INC;MDRNA, Inc., 3830 MONTE VILLA PARKWAY, BOTHELL, WA, 98021-7266, US

(73) Assignee: MDRNA, INC., Bothell, WA (US)

(21) Appl. No.: 12/189,563

(22) Filed: Aug. 11, 2008

Related U.S. Application Data
(60) Provisional application No. 60/955,314, filed on Aug. 10, 2007.
(60) Provisional application No. 60/979,772, filed on Oct. 12, 2007.

(51) Int. Cl. A61K 31/7056; A61K 31/215; A61K 31/351; A61P 31/16; A61K 31/195; A61K 31/13

(52) U.S. Cl. 514/43; 514/529; 514/459; 514/561; 514/662

(57) ABSTRACT

Methods and uses of RNAi-inducing agents for medicaments and treating or preventing a viral infection.

This application claims the benefit under 35 U.S.C. § 119(e) of U.S. Provisional Application Nos. 60/979,772 filed Oct. 12, 2007 and 60/955,314 filed Aug. 10, 2007, the contents of each of which is hereby incorporated by reference in its entirety.

### BACKGROUND

Pathogenic viral infections are some of the most widely spread infections worldwide. For example, a family of such viruses is the influenza family. An estimated 20 to 40 million people died during the 1918 influenza A pandemic. In the United States, about 20 to 40 thousand people die from influenza A virus infection or its complications each year. During epidemics, the number of influenza related hospitalizations may reach over 300,000 in a single winter season.

The influenza viral genome is subject to mutations which cause antigenic shift and drift. These genomic changes are believed to be responsible for pandemic events such as the 1918 swine, 1957 Asian, and 1968 Hong Kong influenza outbreaks, as well as the seasonal reoccurrence of various influenza strains. See, for example, Chapter 45, Fields Virology, Vol. 1, 3^{rd} Edition (1996).

Therapeutics for influenza include neuramidase inhibitors such as oseltamivir, zanamivir, and peramivir. In general, these inhibitors affect virus particle aggregation and release. Antiviral drugs for influenza also include M2 inhibitors, for example amantadine and rimanadine, which are known to inhibit viral replication and prevent the export of the viral genom into the cell nucleus.

A limitation of therapeutics for influenza, including the neuramidase inhibitors, is that the virus can develop resistance to the drugs or treatments. In general, resistance means that a therapeutic agent exhibits a loss of potency toward a resistant virus after exposure of the virus to the therapeutic agent. Further, resistance against one therapeutic agent can correlate to cross-resistance against similar therapeutic agents.

A virus may become resistant to an anti-influenza agent through specific mutations within various genes, for example, the neuraminidase gene or the hemagglutinin gene.

For example, resistance to the anti-influenza agent oseltamivir was observed during treatment of children with oseltamivir. See, for example, Moscona, A. “Neuraminidase inhibitors for influenza,” N Engl J Med 2005; 353:1363-1373; “Avian influenza A (H5N1) infection in humans.” N Engl J Med 2005; 353:1374-1385. Influenza gene mutations correlated to resistance to neuraminidase inhibitors are discussed in Y. Abed, N. Goyette, and G. Bovin, “A reverse genetics study of resistance to neuramidase inhibitors in an influenza A/H1N1 virus,” Antiviral Therapy Vol. 9, pps. 577-81 (2004).

For neuramidase inhibitors such as amantadine and rimantadine, cross-resistance is generally expected. Influenza A variants with reduced in vitro sensitivity to amantadine and rimantadine have been isolated from epidemic strains in areas where adamantane derivatives are being used. Influenza viruses with reduced in vitro sensitivity have been shown to be transmissible and to cause typical influenza illness.

RNA Interference (RNAi) refers to methods of sequence-specific post-transcriptional gene silencing which is mediated by a double-stranded RNA (dsRNA) called a short interfering RNA (siRNA). See Fire, et al., Nature 391:806, 1998, and Hamilton, et al., Science 286:950-951, 1999. RNAi is shared by diverse flora and phyla and is believed to be an evolutionarily-conserved cellular defense mechanism against the expression of foreign genes. See Fire, et al., Trends Genet. 15:358, 1999.

RNAi is therefore a ubiquitous, endogenous mechanism that uses small noncoding RNAs to silence gene expression. See Dykxhoorn, D. M. and J. Lieberman, Annu. Rev. Biomed. Eng. 8:377-402, 2006. RNAi can regulate important genes involved in cell death, differentiation, and development. RNAi may also protect the genome from invading genetic elements, encoded by transposons and viruses. When a siRNA is introduced into a cell, it binds to the endogenous RNAi machinery to disrupt the expression of mRNA containing complementary sequences with high specificity. Any disease-causing gene and any cell type or tissue can potentially be targeted. This technique has been rapidly utilized for gene-function analysis and drug-target discovery and validation. Harnessing RNAi also holds great promise for therapy, although introducing siRNAs into cells in vivo remains an important obstacle.

The mechanism of RNAi, although not yet fully characterized, is through cleavage of a target mRNA. The RNAi response involves an endonuclease complex known as the RNA-induced silencing complex (RISC), which mediates cleavage of a single-stranded RNA complementary to the antisense strand of the siRNA duplex. Cleavage of the target RNA takes place in the middle of the region complementary to the antisense strand of the siRNA duplex (Elbashir, et al., Genes Dev. 15:188, 2001).

One way to carry out RNAi is to introduce or express a siRNA in cells. Another way is to make use of an endogenous ribonuclease III enzyme called dicer. One activity of dicer is to process a long dsRNA into siRNAs. See Hamilton, et al., Science 286:950-951, 1999; Berstein, et al., Nature 409:363, 2001. A siRNA derived from dicer is typically about 21-23 nucleotides in overall length with about 19 base pairs duplexed. See Hamilton, et al., supra; Elbashir, et al., Genes Dev. 15:188, 2001. In essence, a long dsRNA can be introduced in a cell as a precursor of a siRNA.

What is needed are compositions, medicaments, and their uses in modalities for effective therapeutics in treating, ameliorating, or preventing pathogenic viral infections, diseases and disorders.

### BRIEF SUMMARY

This disclosure encompasses a method for preventing or treating an influenza infection in a subject caused by a drug resistant strain of influenza comprising administering to the subject a therapeutically-effective amount of one or more RNAi-inducing agents having efficacy against the drug resistant strain.

In one aspect, the drug resistant strain may be resistant to an anti-viral drug. In certain aspects, the anti-viral drug may be a neuramidase inhibitor drug, for example, oseltamivir, zanamivir, and peramivir. In another aspect, the anti-viral drug is an M2 inhibitor, for example amantadine or rimantadine. In yet another aspect, the anti-viral drug is a amantadine or ribavarin.

In other aspects, the one or more RNAi-inducing agents may be administered by intranasal delivery to a subject. In certain aspects, the dose of the one or more RNAi-inducing agents may be from about 0.001 mg/kg to about 2 mg/kg or about 0.006 mg/kg to about 0.6 mg/kg.

In other aspects, the one or more RNAi-inducing agents may be administered by pulmonary delivery to a subject. In a related aspect, the dose of the one or more RNAi-inducing agents may be from about 0.001 mg/kg to about 5 mg/kg or from about 1 mg/kg to about 4 mg/kg.

This disclosure encompasses a method for preventing or treating an influenza infection in a subject in need thereof comprising administering to the subject a therapeutically-effective amount of one or more RNAi-inducing agents in combination with a neuramidase inhibitor drug. In one aspect, the one or more RNAi-inducing agents and the neuramidase inhibitor drug may be administered in series. In a related aspect, the subject may have used the neuramidase inhibitor within 24 hours of the administration of the RNAi-inducing agents.

This disclosure encompasses, a method for preventing or treating an influenza infection in a subject in need thereof comprising administering to the subject a therapeutically-effective amount of one or more RNAi-inducing agents having therapeutic efficacy against at least 90% of influenza viruses. In one aspect, the one or more RNAi-inducing agents may have therapeutic efficacy against influenza A, influenza B, and highly pathogenic influenza viruses, for example H1N1, H3N2, and H5N1.

This disclosure encompasses, a method for preventing or treating an influenza infection in a subject in need thereof comprising administering to the subject a therapeutically-effective amount of one or more RNAi-inducing agents which delay the emergence of a resistant influenza strain that is resistant to an RNAi-inducing agent, which is different from the one or more RNA-inducing agents. In certain aspects, the one or more RNAi-inducing agents may delay the emergence of the influenza strain by at least one or more passages in vitro or by at least two or more passages in vitro.

This disclosure encompasses, a method for preventing or treating an influenza infection in a subject in need thereof comprising administering to the subject a therapeutically-effective amount of two or more RNAi-inducing agents, wherein the two or more RNAi-inducing agents are targeted to different portions of the influenza genome and are administered in series. In certain aspects, the two or more RNAi-inducing agents may be targeted to a portion of an NP influenza gene or a PA influenza gene, or targeted to different portions of an NP influenza gene or a PA influenza gene, or targeted to a portion of an NP influenza gene and at least one of the RNAi-inducing agents is targeted to a portion of a PA influenza gene or a PB1 influenza gene.

This disclosure encompasses, a method for preventing or treating an influenza infection in a subject in need thereof comprising administering to the subject a therapeutically-effective amount of one or more RNAi-inducing agents in combination with a neuramidase inhibitor drug. In one aspect, the one or more RNAi-inducing agents and the neuramidase inhibitor drug may be administered in series. In another aspect, the subject may have used a neuramidase inhibitor within 24 hours of the administration of the one or more RNAi-inducing agents. In yet another aspect, the amount of the neuramidase inhibitor drug administered to the subject may be less than that amount that would have been indicated for treating or preventing the influenza infection in the subject by use of the neuramidase inhibitor drug alone in the absence of the one or more RNAi-inducing agents.

In certain aspects, the RNAi-inducing agents may be DX3030, DX3044, DX4046, DX3048, DX3050, and peptide conjugates thereof.

In general, this disclosure encompasses methods for preventing or treating an influenza infection in a subject by administering to the subject a therapeutically-effective amount of one or more RNAi-inducing agents having a broad spectrum of efficacy against seasonal influenza and highly pathogenic influenza.

This disclosure encompasses methods for preventing or treating an influenza infection in a subject caused by a drug resistant strain of influenza by administering to the subject a therapeutically-effective amount of one or more RNAi-inducing agents having efficacy against the drug resistant strain. The drug resistant strain may result from use of a neuramidase inhibitor drug such as oseltamivir.

This disclosure encompasses methods for preventing or treating an influenza infection in a subject by administering to the subject a therapeutically-effective amount of one or more RNAi-inducing agents which delay the emergence of influenza strains that are resistant to the RNAi-inducing agents.

This disclosure encompasses methods for preventing or treating an influenza infection in a subject by administering to the subject a therapeutically-effective amount of two or more RNAi-inducing agents wherein the RNAi-inducing agents are targeted to different portions of the influenza genome and are administered in series.

This disclosure encompasses methods for preventing or treating an influenza infection in a subject by administering to the subject a therapeutically-effective amount of one or more RNAi-inducing agents in combination with a neuramidase inhibitor drug or an oseltamivir drug.

### BRIEF DESCRIPTION OF THE DRAWINGS

FIG. 1. BALB/c mice were treated intranasally with indicated amounts of NP specific siRNA in PBS or PBS control. Two hours later, all mice were infected intranasally (1000 pfu/mouse) with the PR8 serotype. The lungs were harvested 24 hours post-infection, and viral titer was measured from lung homogenates by MDCK-HA assay. P values between PBS and siRNA groups indicate statistical significance with 0.5, 1 and 2 mg/kg siRNA treated groups.

FIG. 2. BALB/c mice were administered control and NP-targeting siRNA intranasally (10 mg/kg, in PBS). Three hours later all the mice were infected i.n. with PR8 virus (50 pfu/mouse). The lungs were harvested at 24 and 48 hours post-infection and total RNA was isolated from the left lung. Total mRNA was reverse transcribed to cDNA using dT18 primers (SEQ ID NO: 221). Real time PCR was carried out using PB1 specific primers to quantify viral mRNA levels. GAPDH was used as an internal control. The right and middle lungs were homogenized and the viral titer was measured by MDCK-HA assay. The virus titer in the samples at 48 hours post-infection is shown in the figure (statistic significance was found between PBS and NP siRNA treated group using student t test (p=0.01); the titer in the samples 24 hours post-infection was too low to detect, possibly due to siRNA directed suppression.

FIG. 3. BALB/c mice were treated intranasally with 10 mg/kg cyclophilin B specific siRNA or GFP siRNA in PBS or PBS control. There were five mice per group. The mouse lungs were harvested 24 hours later. Total RNA was purified from the lung samples and reverse transcription was conducted using dT18 primer (SEQ ID NO: 221). Cyclophilin B-specific primers were used in real-time PCR to quantify the target mRNA level. GAPDH-specific primers were also used in the PCR reaction as control.

### DETAILED DESCRIPTION OF THE DISCLOSURE

This disclosure relates generally to the fields of RNA interference and delivery of RNA therapeutics. More particularly, this disclosure relates to compositions and formulations for ribonucleic acids, and their uses for medicaments and for delivery as therapeutics. This disclosure relates generally to methods of using ribonucleic acids in RNA interference for gene-specific inhibition of gene expression in mammals.

This disclosure is based in part on the phenomenon of RNA interference (RNAi) wherein the presence in a cell of a double-stranded RNA (dsRNA) containing a portion that is complementary to a target RNA inhibits expression of the target RNA in a sequence-specific manner. Generally, inhibition is caused by cleavage of the target or inhibition of its translation.

This disclosure also encompasses anti-influenza agents. Influenza viruses are enveloped, negative-stranded RNA viruses of the Orthomyxoviridae family which are generally classified as influenza types A, B, and C.

This disclosure encompasses the use of dsRNAs targeted to various viral genes including viral nucleoprotein sequences, to disrupt viral pathways and inhibit viral replication in an infected cell. Thus, RNAi can be an effective mechanism to reduce viral titers and infection in an animals and humans.

This disclosure provides a range of compositions, formulations and methods which includes an interfering nucleic acid or a precursor thereof in combination with various components including lipids, lipoid moieties, peptides, natural or synthetic polymers, and conjugate moieties thereof.

In some aspects, this disclosure provides compositions to facilitate the delivery of RNAi-inducing agents to cells, tissues, organs, and in living animals, for example, mammals and humans.

The term “dsRNA” as used herein refers to any nucleic acid molecule comprising at least one ribonucleotide molecule and capable of inhibiting or down regulating gene expression, for example, by promoting RNA interference (“RNAi”) or gene silencing in a sequence-specific manner. The dsRNAs of this disclosure may be suitable substrates for Dicer or for association with RISC to mediate gene silencing by RNAi. One or both strands of the dsRNA can further comprise a terminal phosphate group, such as a 5′-phosphate or 5′, 3′-diphosphate. As used herein, dsRNA molecules, in addition to at least one ribonucleotide, can further include substitutions, chemically-modified nucleotides, and non-nucleotides. In certain embodiments, dsRNA molecules comprise ribonucleotides up to about 100% of the nucleotide positions.

Examples of dsRNA molecules can be found in, for example, U.S. patent application Ser. No. 11/681,725, U.S. Pat. Nos. 7,022,828 and 7,034,009, and PCT International Application Publication No. WO/2003/070897. The entire contents of the above identified patent applications and patents are hereby incorporated by reference.

In addition, as used herein, the terms “dsRNA,” “RNAi-inducing agent,” and “RNAi-agent” are meant to be synonymous with other terms used to describe nucleic acid molecules that are capable of mediating sequence specific RNAi including meroduplex RNA (mdRNA), nicked dsRNA (ndsRNA), gapped dsRNA (gdsRNA), short interfering nucleic acid (siNA), siRNA, microRNA (miRNA), short hairpin RNA (shRNA), short interfering oligonucleotide, short interfering substituted oligonucleotide, short interfering modified oligonucleotide, chemically-modified dsRNA, and post-transcriptional gene silencing RNA (ptgsRNA), among others.

The term “large double-stranded (ds) RNA” refers to any double-stranded RNA longer than about 40 base pairs (bp) to about 100 bp or more, particularly up to about 300 bp to about 500 bp. The sequence of a large dsRNA may represent a segment of an mRNA or an entire mRNA. A double-stranded structure may be formed by self-complementary nucleic acid molecule or by annealing of two or more distinct complementary nucleic acid molecule strands.

In some aspects, a dsRNA comprises two separate oligonucleotides, comprising a first strand (antisense) and a second strand (sense), wherein the antisense and sense strands are self-complementary (i.e., each strand comprises a nucleotide sequence that is complementary to a nucleotide sequence in the other strand and the two separate strands form a duplex or double-stranded structure, for example, wherein the double-stranded region is about 10 to about 24 base pairs, 15 to about 24 base pairs or about 26 to about 40 base pairs); the antisense strand comprises a nucleotide sequence that is complementary to a nucleotide sequence in a target nucleic acid molecule or a portion thereof (e.g., a human mRNA); and the sense strand comprises a nucleotide sequence corresponding (i.e., homologous) to the target nucleic acid sequence or a portion thereof (e.g., a sense strand of about 15 to about 25 nucleotides or about 26 to about 40 nucleotides corresponds to the target nucleic acid or a portion thereof).

In some aspects, the dsRNA may be assembled from a single oligonucleotide in which the self-complementary sense and antisense strands of the dsRNA are linked together by a nucleic acid based-linker or a non-nucleic acid-based linker. In some embodiments, the first (antisense) and second (sense) strands of the dsRNA molecule are covalently linked by a nucleotide or non-nucleotide linker as described herein and known in the art. In some embodiments, a first dsRNA molecule is covalently linked to at least one second dsRNA molecule by a nucleotide or non-nucleotide linker known in the art, wherein the first dsRNA molecule can be linked to a plurality of other dsRNA molecules that can be the same or different, or any combination thereof. In some embodiments, the linked dsRNA may include a third strand that forms a meroduplex with the linked dsRNA.

In some respects, dsRNA molecules described herein form a meroduplex RNA (mdRNA) having three or more strands, for example, an ‘A’ (first or antisense) strand, ‘S1’ (second) strand, and ‘S2’ (third) strand in which the ‘S1’ and ‘S2’ strands are complementary to and form base pairs (bp) with non-overlapping regions of the ‘A’ strand (e.g., an mdRNA can have the form of A:S1S2). The S1, S2, or more strands together essentially comprise a sense strand to the ‘A’ strand. The double-stranded region formed by the annealing of the ‘S1’ and ‘A’ strands is distinct from and non-overlapping with the double-stranded region formed by the annealing of the ‘S2’ and ‘A’ strands. An mdRNA molecule is a “gapped” molecule, meaning a “gap” ranging from 0 nucleotides up to about 10 nucleotides. In some embodiments, the A:S1 duplex is separated from the A:S2 duplex by a gap resulting from at least one unpaired nucleotide (up to about 10 unpaired nucleotides) in the ‘A’ strand that is positioned between the A:S1 duplex and the A:S2 duplex and that is distinct from any one or more unpaired nucleotide at the 3′-end of one or more of the ‘A’, ‘S1’, or ‘S2’ strands. In some embodiments, the A:S1 duplex is separated from the A:B2 duplex by a gap of zero nucleotides (i.e., a nick in which only a phosphodiester bond between two nucleotides is broken or missing in the polynucleotide molecule) between the A:S1 duplex and the A:S2 duplex—which can also be referred to as nicked dsRNA (ndsRNA). For example, A:S1S2 may be comprised of a dsRNA having at least two double-stranded regions that combined total about 14 base pairs to about 40 base pairs and the double-stranded regions are separated by a gap of about 0 to about 10 nucleotides, optionally having blunt ends, or A:S1S2 may comprise a dsRNA having at least two double-stranded regions separated by a gap of up to 10 nucleotides wherein at least one of the double-stranded regions comprises between about 5 base pairs and 13 base pairs.

As described herein, a dsRNA molecule which contains three or more strands may be referred to as a “meroduplex” RNA (mdRNA). Examples of mdRNA molecules can be found in U.S. Provisional Patent Application Nos. 60/934,930 and 60/973,398, and International Patent Application No. PCT/US07/081836. The entire contents of the above identified patent applications are hereby incorporated by reference.

A dsRNA or large dsRNA may include a substitution or modification in which the substitution or modification may be in a phosphate backbone bond, a sugar, a base, or a nucleoside. Such nucleoside substitutions can include natural non-standard nucleosides (e.g., 5-methyluridine or 5-methylcytidine or a 2-thioribothymidine), and such backbone, sugar, or nucleoside modifications can include an alkyl or heteroatom substitution or addition, such as a methyl, alkoxyalkyl, halogen, nitrogen or sulfur, or other modifications known in the art.

In addition, as used herein, the term “RNAi” is meant to be equivalent to other terms used to describe sequence specific RNA interference, such as post transcriptional gene silencing, translational inhibition, or epigenetics. For example, dsRNA molecules of this disclosure can be used to epigenetically silence genes at the post-transcriptional level or the pre-transcriptional level or any combination thereof.

In some aspects, this disclosure provides compositions containing one or more RNAi-inducing agents which are targeted to one or more genes or target transcripts, along with one or more delivery components. Examples of delivery components include lipids, peptides, polymers, polymeric lipids, conjugates, and complexes thereof.

The compositions and formulations of this disclosure may be used for delivery of RNAi-inducing entities such as dsRNA, siRNA, mdRNA, miRNA, shRNA, or RNAi-inducing vectors to cells in intact mammalian subjects, and may also be used for delivery of these agents to cells in culture.

This disclosure also provides methods for the delivery of one or more RNAi-inducing agents or entities to cells, organs and tissues within the body of a mammal. In some respects, compositions containing an RNAi-inducing entity may be introduced by various routes to be transported within the body and taken up by cells in one or more organs or tissues, where expression of a target transcript is modulated.

In general, this disclosure encompasses RNAi-inducing agents that are useful therapeutics to prevent and treat diseases or disorders characterized by various aberrant processes. For instance, viruses that infect mammals can replicate by taking control of cellular machinery of the host cell. Thus, dsRNAs are useful in disrupting viral pathways which control virus production (e.g., viral replication, assembly, and/or release).

This disclosure includes methods for treating or preventing a viral infection in a subject by use of one or more therapeutic RNAi-inducing agents having a broad spectrum of efficacy against multiple strains of a target virus. An RNAi-inducing agent of this disclosure can be targeted to a sequence of a viral gene in a known variant strain or variants of a virus, and exhibit sequence-specific gene silencing of the targeted viral gene in those variants. For example, an RNAi-inducing agent may be targeted to, and exhibit efficacy against a seasonal strain, a highly pathogenic strain, a clinical strain, and/or a subclinical strain of influenza virus, as well as variant strains of influenza. In general, not limiting examples of influenza strains include H1N₂, H1N1, H7N₇, H5N₁, H7N₃, H9N₂, H7N₂, H3N₂, and variants thereof.

Examples of suitable RNAi-inducing agents for use in the present disclosure are described in U.S. patent application Ser. No. 11/687,564, the contents of which are hereby incorporated by reference in its entirety.

In some embodiments, an RNAi-inducing agent targeted to a conserved sequence of a viral gene in a known variant or variants of a virus can advantageously exhibit efficacy against other strains of the virus. Other strains of a virus may exist in various subjects or populations, or may emerge in various subjects or populations as a strain that is resistant to a drug.

The emergence of a resistant viral strain may result from selection pressure due to an antiviral agent, including an RNAi-inducing agent. In some embodiments, an RNAi-inducing agent targeted to a sequence of a viral gene in a known variant or variants of a virus can advantageously delay the emergence of a resistant strain of the virus.

In some embodiments, a use of RNAi-inducing agents encompasses administrating the RNAi-inducing agents to a subject prior to the subject showing any clinical signs or illness of a viral infection.

In some aspects, the RNAi-inducing agents are administrated throughout the duration of influenza activity in the community.

Uses of RNAi-inducing agents and medicaments for treatment modalities contemplated in this disclosure include the use or administration of RNAi-inducing agents in series. A use of RNAi-inducing agents in series includes modalities in which one RNAi-inducing agent is used, followed by the use of a different RNAi-inducing agent.

The time between the use of a first RNAi-inducing agent and a second RNAi-inducing agent in series may be 1, 2, 3, 4, 5, 6, 8, 9, 10, 12 hours or more. In some embodiments, the time between the use of a first RNAi-inducing agent and a second RNAi-inducing agent in series may be 1, 2, 3, 4, 5, or 6 days or more.

In some aspects, a use of RNAi-inducing agents in series encompasses alternating uses of different RNAi-inducing agents. For example, a use in series includes the use of a first RNAi-inducing agent, followed by the use of a second RNAi-inducing agent, which in turn is followed by another use of the first RNAi-inducing agent, or by the use of an additional RNAi-inducing agent.

In some aspects, a use of RNAi-inducing agents in series includes modalities in which one or more RNAi-inducing agent is used, whereby each RNAi-inducing agent used may have a different nucleotide sequence (e.g., each RNAi-inducing agent targets a different viral gene or different regions of the same viral gene) or each RNAi-inducing agent used may have an overlapping nucleotide sequence (i.e., the RNAi-inducing agents target an overlapping region of a target gene) or each RNAi-inducing agent may have a different type of nucleotide modification, or any combination thereof.

In some embodiments, a use of RNAi-inducing agents in series or parallel encompasses administrating the RNAi-inducing agents to a subject prior to the subject showing any clinical signs or illness of a viral infection.

In some aspects, the RNAi-inducing agents in series or parallel are administered throughout the duration of influenza activity in the community.

In some embodiments, a use of RNAi-inducing agents in series or parallel encompasses administrating the RNAi-inducing agents to a subject after the subject begins showing clinical signs or illness of a viral infection or the subject having detectable levels of virus in the blood as assayed by any method to one skilled in the art (e.g., RT-PCR, Northern blot, ELISA, or any other method described herein). In some aspects, the time between the subject exhibiting clinical signs or illness of a viral infection or the subject having detectable levels of virus in the blood and the administration of the RNAi-inducing agents in series or parallel may be within from about 1 hour to about 72 hours, or within about 6 hours to about 48 hours, or within about 12 hours to about 36 hours, or within about 24 hours.

In some aspects the time between the subject exhibiting clinical signs or illness of a viral infection or the subject having detectable levels of virus in the blood and the administration of the RNAi-inducing agents in series or parallel may be 1, 2, 3, 4, 5, 6, 7, 8, 9, 10 or more days.

A use of an RNAi-inducing agent of this disclosure includes the use by a subject, or the administration of the RNAi-inducing agent to the subject in combination with the use or administration of an antiviral agent. Non-limiting examples of antiviral agents include a neuramidase inhibitor (e.g., oseltamivir, zanamivir, and peramivir), an M2 inhibitor (e.g., amantadine and rimantadine), amantiadine, and ribavirin.

A dosage of such antiviral agents used in combination with one or more RNAi-inducing agents of this disclosure may be from about 0.01 mg to about 200 mg daily, or about 1 mg to about 150 mg daily, or about 5 mg to about 140 mg daily. Dosage amounts of RNAi-inducing agents that may be used within the present disclosure may include from about 0.01 mg/kg to about 10 mg/kg, or from about 1 mg/kg to about 4 mg/kg, or from about 0.06 mg/kg to about 2 mg/kg body weight of the subject. Doses may be administered to a subject 1, 2, 3, 4, or more times per day.

Uses of RNAi-inducing agents and medicaments in combination with an antiviral agent for treatment modalities contemplated in this disclosure include the use or administration of RNAi-inducing agents and one or more antiviral agents in series. A use of RNAi-inducing agents in series includes modalities in which one or more RNAi-inducing agents is used, followed by the use of one or more antiviral agents.

Uses of RNAi-inducing agents and medicaments in combination with an antiviral agent for treatment modalities contemplated in this disclosure include the use or administration of RNAi-inducing agents and one or more antiviral agents in parallel.

In some embodiments, the amount of an antiviral agent (e.g., a neuramidase inhibitor) used or administered concurrently with an RNAi-inducing agent may be less than that amount which would have been indicated for preventing or treating a viral infection in a subject by use of the neuramidase inhibitor alone in the absence of the RNAi-inducing agent.

A use of an RNAi-inducing agent in combination with the use or administration of a neuramidase inhibitor includes uses or embodiments in which a subject may be administered an RNAi-inducing agent and a neuramidase inhibitor in sequentially alternating series or doses.

The time between the use of one or more RNAi-inducing agents and one or more antiviral gents in series may be about 1, 2, 3, 4, 5, 6, 8, 9, 10, 12 hours or more. In some embodiments, the time between the use of one or more RNAi-inducing agents and one or more antiviral agents in series may be about 1, 2, 3, 4, 5, 6, 7, 8, 9, 10 days or more.

In some embodiments, a use of RNAi-inducing agents in combination with an antiviral agent either in series or parallel encompasses administrating the RNAi-inducing agents in combination with an antiviral agent either in series or parallel to a subject prior to the subject showing any clinical signs or illness of a viral infection.

In some embodiments, a use of RNAi-inducing agents in combination with an antiviral agent for treatment modalities contemplated in this disclosure include administrating the RNAi-inducing agents in combination with an antiviral agent in series or parallel to a subject after the subject begins showing clinical signs or illness of a viral infection or the subject having detectable levels of virus in the blood. In some aspects, the time between the subject exhibiting clinical signs or illness of a viral infection or the subject having detectable levels of virus in the blood and the administration of the RNAi-inducing agents in combination with an antiviral agent in series or parallel may be within from about 1 hour to about 72 hours, or about 6 hours to about 48 hours, or about 12 hours to about 36 hours, or about 24 hours.

In some aspects, the time between the subject exhibiting clinical signs or illness of a viral infection or the subject having detectable levels of virus in the blood and the administration of the RNAi-inducing agents in combination with an antiviral agent either in series or parallel may be 1, 2, 3, 4, 5, 6, 7, 8, 9, 10 or more days.

In some embodiments, a use of one or more RNAi-inducing agents in combination with one or more antiviral agents for treatment modalities contemplated in this disclosure include administrating the RNAi-inducing agents in series or parallel, as described above, and the one or more antiviral agents in series or parallel, as describe above, to a subject.

This disclosure also contemplates uses and methods for inhibiting replication of a drug resistant respiratory virus by RNA interference in a mammalian cell or subject.

### Broad Spectrum RNAi Therapeutics

RNAi-inducing agents of this disclosure may advantageously have a broad spectrum of efficacy against various strains of a target virus.

A goal for influenza therapy is to have RNAi-inducing agents that are effective against a broad spectrum of a patient population.

For influenza, an RNAi-inducing agent may be targeted to, and exhibit efficacy against a seasonal strain of influenza virus, as well as variants of the seasonal strain and other variant strains of influenza.

In some embodiments, an RNAi-inducing agent contemplated in this disclosure has efficacy or has therapeutic efficacy against at least 10%, 20%, 30,%, 40%, 50%, 60%, 70%, 80%, 90% or more influenza viruses.

In some embodiments, an RNAi-inducing agent can be targeted to a sequence of a viral gene in a known variant or variants of a virus and advantageously provide antiviral activity against other strains of the virus. Other strains of a virus may exist in various subjects or populations, or may emerge in various subjects or populations as a strain that is resistant to a drug.

For example, a drug resistant strain of influenza may result from use of a neuramidase inhibitor drug such as oseltamivir in a subject or population. In some embodiments of this disclosure, an RNAi-inducing agent targeted to a sequence of an influenza gene in a known variant or variants of an influenza virus can advantageously retain efficacy against strains of the virus that are resistant to an antiviral neuramidase inhibitor or M2 ion channel inhibitors.

### RNAi Therapeutics for Drug Resistant Influenza

In an individual patient, it is possible for viral resistance to a drug to develop after even a single dose of a drug. In particular, resistance may develop when the drug is used by subjects who are not in need of treatment, or where the drug is inappropriately prescribed and dosed.

As used herein, “resistance”, “drug resistance”, “drug resitant virus”, “drug resistant variant”, “drug resistant strain” is used to refer to a therapeutic agent, drug or anti-influenza agent meant to inhibit or prevent viral infection that exhibits a loss of potency toward inhibiting or preventing viral infection after exposure of the virus to the therapeutic agent, drug, or anti-influenza agent.

A limitation of drugs for influenza, including the neuramidase inhibitors, is that the virus can develop resistance to the drugs or treatments. Further, a resistance or drug resistance against one therapeutic agent or drug may correlate to cross-resistance against other therapeutic agents or drugs.

A virus may become resistant to an anti-influenza agent through specific mutations within the various genes, for example, the neuraminidase gene or the hemagglutinin gene.

For example, resistance to the anti-influenza agent oseltamivir was observed during treatment of children with oseltamivir. See, for example, Moscona, A. “Neuraminidase inhibitors for influenza,” N Engl J Med 2005; 353:1363-1373; “Avian influenza A (H5N1) infection in humans.” N Engl J Med 2005; 353:1374-1385. Influenza gene mutations correlated to resistance to neuraminidase inhibitors are discussed in Y. Abed, N. Goyette, and G. Bovin, “A reverse genetics study of resistance to neuramidase inhibitors in an influenza A/H1N1 virus,” Antiviral Therapy Vol. 9, pps. 577-81 (2004).

For neuramidase inhibitors such as amantadine and rimantadine, cross-resistance is generally expected. Influenza A variants with reduced in vitro sensitivity to amantadine and rimantadine have been isolated from epidemic strains in areas where adamantane derivatives are being used. Influenza viruses with reduced in vitro sensitivity have been shown to be transmissible and to cause typical influenza illness.

Pressures that can induce resistant strains of a virus to emerge include inappropriate use, for example, long-term use, use of lowered doses to extend the course of treatment with a limited supply, intermittent or infrequent use below the prescribed dose or frequency, or lack of an age- and weight-tailored treatment regimen.

For example, populations that are in close contact during influenza season pose a threat for emergence and propagation of resistant strains. For example, hospitals and care facilities, schools, military, and other high population density conditions.

Other factors that could affect the emergence of resistance include use by high risk groups such as children below 5 years of age, or use by immune-compromised patients.

In general, children can have a long infective period and can carry and transmit resistant virus.

Influenza strains are reported at The Influenza Sequence Database, Macken, C., Lu, H., Goodman, J., and Boykin, L., “The value of a database in surveillance and vaccine selection,” in Options for the Control of Influenza IV. A.D.M.E., Osterhaus, N. Cox and A. W. Hampson (eds.) Science, 2001, 103-106.

The extensive use of a single anti-influenza agent can induce resistance. Availability of alternative therapeutics is desirable. Use of a dsRNA therapeutic of this disclosure advantageously provides an alternative to reduce the emergence and spread of resistant strains.

In general, once a resistant strain is known or suspected to be in circulation, all patients are at risk. In that case, medical practice for viral infection generally dictates that new patients be started on an alternative therapy. Further, patients using the resistance-causing agent should be switched to an alternative therapeutic.

A goal for influenza therapy is to have a treatment for a broad spectrum of the patient population. An anti-viral RNAi therapeutic of this disclosure can be advantageously effective against drug resistant influenza strains. The dsRNA therapeutics of this disclosure may not be subject to cross-resistance because the sequence-specific targeting of the dsRNA avoids the viral mutations responsible for resistance to other drugs. Moreover, multiple dsRNA therapeutics of this disclosure may be utilized to avoid any resistance that may develop to one single dsRNA.

A dsRNA influenza medicament of this disclosure would be useful for any population using the resistance-causing agent, as well as any patient exposed to a resistant strain. At the same time, the dsRNA influenza medicament can be prescribed to patients for non-resistant strains.

In some embodiments, a dsRNA influenza agent of this disclosure is useful in a medicament for prophylaxis in patients post-exposure to a known or suspected strain of a resistant virus. In some embodiments, the medicament is useful during the period of exposure.

In some aspects, a dsRNA influenza agent of this disclosure is useful in a medicament for treatment of active infection in patients. In some embodiments, the medicament is useful within one or two days after onset of symptoms of influenza infection, or before onset of symptoms.

In some aspects, the dsRNA RNAi influenza agents of this disclosure are useful in preparation of medicaments for patients including the healthy individuals, immuno-compromised patients such as those having organ transplants, undergoing chemotherapy, or HIV-AIDS patients, as well as patients exposed to seasonal, resistant, or pandemic influenza strains.

In some embodiments, the dose regimen is at least once daily for about 1 to 20 days, or for about 3 to 10 days.

### Antiviral Combination Therapeutics

The diversity of a viral genome exhibited over the course of an infection in general makes combination therapy a useful approach for antiviral agents. In combination therapy it may be advantageous to delay emergence of resistant strains.

A use of an RNAi-inducing agent of this disclosure includes the use by a subject, or the administration of the RNAi-inducing agent to the subject in combination with the use or administration of other antiviral agents.

Antiviral medications with activity against influenza viruses for the prevention and treatment of influenza include the neuraminidase inhibitors oseltamivir and zanamivir having activity against both influenza A and B viruses. Oseltamivir and zanamivir are used as chemoprophylaxis agents.

Another class of influenza antiviral medications are the adamantanes, amantadine and rimantadine, used for the treatment and prevention of influenza. However, a high proportion of circulating influenza viruses in the U.S. in recent years have been resistant to the adamantanes. Thus, the use of amantadine and rimantadine may not be recommended for the treatment or chemoprophylaxis of influenza in a particular influenza season.

In some embodiments, methods for preventing or treating an influenza infection encompass the use by a subject of a neuramidase inhibitor within 24 or 48 hours of the use or administration of an RNAi-inducing agent.

In some embodiments, the amount of a neuramidase inhibitor used in combination with, or administered concurrently with an RNAi-inducing agent may be less than that amount which would have been indicated for preventing or treating a viral infection in a subject by use of the neuramidase inhibitor alone in the absence of the RNAi-inducing agent.

A use of an RNAi-inducing agent in combination with the use or administration of a neuramidase inhibitor includes uses or embodiments in which a subject may be administered an RNAi-inducing agent and a neuramidase inhibitor in series.

In some embodiments, a subject may be administered a dsRNA followed by a neuramidase inhibitor in series after at least 1, 2, 3, 4, 5, 6, 8, 10, 12 hours or more. In some embodiments, a use of an RNAi-inducing agent and a neuramidase inhibitor in series encompasses the use of an RNAi-inducing agent followed by a neuramidase inhibitor after 1, 2, 3, 4, 5, or 6 days or more.

### RNA Therapeutics and Structures

As used herein, a nucleic acid may include naturally occurring nucleosides (e.g., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose).

As used herein, the terms “RNA” and “DNA” include naturally produced as well as laboratory produced materials. The terms “target mRNA” and “target transcript” are synonymous as used herein.

The terms “RNA interference,” “RNAi,” and “gene silencing” refer to selective intracellular modulation of RNA involved in endogenous or exogenous gene expression, including production of RNAi agents intracellularly, for example, from a plasmid or transgene, or by introduction of precursors of RNAi agents which are processed by dicer enzyme, to modulate or silence the expression of one or more target genes.

The terms “small interfering RNA,” “siRNA” and “short interfering RNA” refer to an RNA or RNA analog comprising from about 10-60 nucleotides or nucleotide analogs that is capable of initiating, directing and/or mediating RNA interference. Generally, as used herein the term “siRNA” refers to double stranded siRNA having a sense strand and an antisense strand.

The term “short hairpin RNA” (“shRNA”) refers to a precursor of an siRNA or siRNA analog that can provide an siRNA or siRNA analog. An shRNA is typically folded into a hairpin structure and contains a single stranded or “loop” portion of at least one nucleotide. For example, an shRNA may be described as an RNA molecule that contains at least two complementary portions hybridized or capable of hybridizing to form a double-stranded or duplex structure sufficiently long to mediate RNAi, as described above for siRNA duplexes, and at least one single-stranded portion, typically from 1 to about 10 nucleotides in length that forms a loop connecting the regions of the shRNA that form the duplex portion. The duplex portion may, but typically does not, contain one or more mismatches and/or one or more bulges consisting of one or more unpaired nucleotides in either or both strands. shRNAs are capable of inhibiting expression of a target transcript that is complementary to a portion of the shRNA, referred to as the antisense or guide strand of the shRNA.

In some embodiments of this disclosure, the 5′ end of an shRNA has a phosphate group. In some embodiments of this disclosure, the 3′ end of an shRNA has a hydroxyl group.

The terms “RNAi-inducing agent” or “RNAi agent” include dicer-processed precursors such as dicer substrates and dicer substrate conjugates which are capable of initiating, directing and/or mediating RNA interference.

Dicer substrate conjugates are disclosed in U.S. Patent Application No. 60/945,868. Dicer substrate conjugates employ an interfering ribonucleic acid, or a precursor thereof, in combination with a polynucleotide delivery-enhancing polypeptide. The polynucleotide delivery-enhancing polypeptide may be a natural or artificial polypeptide selected for its ability to enhance intracellular delivery or uptake of polynucleotides, including interfering RNAs and their precursors.

Dicer substrate conjugates encompasses polynucleotide delivery-enhancing polypeptides conjugated to dicer-active dsRNAs. As used herein, the term “dicer substrate” refers to a dicer-active dsRNA, which is a dsRNA that is capable of being processed by dicer ribonuclease. Dicer-active dsRNA peptide conjugates of this disclosure can be used as novel therapeutic pro-drug delivery systems in the treatment of disease. These dicer-active dsRNA peptide conjugates function analogous to a pro-drug or precursor siRNA in that upon delivery into a cell, the dsRNA peptide conjugate can be processed and cleaved by dicer, whereupon an siRNA is liberated that is capable of loading into the RISC complex. The liberated siRNA may then enter the RISC complex to effect post-transcriptional gene silencing. Thus, the dicer-active dsRNA peptide conjugate, and the dicer-liberated siRNA are RNAi-inducing agents.

A dicer substrate peptide conjugate may contain a double stranded ribonucleic acid (dsRNA) having a sense strand and an antisense strand and a double-stranded region of from 25 to 30 base pairs, and a peptide comprising from about 5 to about 100 amino acids, wherein the dsRNA is conjugated to the peptide.

In some embodiments of this disclosure, an siRNA contains a strand that inhibits expression of a target RNA via a translational repression pathway utilized by endogenous small RNAs referred to as microRNAs. In certain embodiments of the disclosure an shRNA may be processed intracellularly to generate an siRNA that inhibits expression of a target RNA via this microRNA translational repression pathway. Any “target RNA” may be referred to as a “target transcript” regardless of whether the target RNA is a messenger RNA. The terms “target RNA” and “target transcript” are used interchangeably herein. The term RNAi-inducing agent encompasses RNAi agents and vectors other than naturally occurring molecules not modified or transported by the hand of man whose presence within a cell results in RNAi.

An “RNAi-inducing vector” includes a vector whose presence within a cell or fusion to a cell leads to transcription of one or more RNAs that self-hybridize or hybridize to each other to form an RNAi agent. In various embodiments of the disclosure this term encompasses plasmids, for example, DNA vectors whose sequence may comprise sequence elements derived from a virus, or viruses. In general, the vector comprises a nucleic acid operably linked to an expression signal or signals so that one or more RNA molecules that hybridize or self-hybridize to form an RNAi agent is transcribed when the vector is present within a cell. Thus the vector provides a template for intracellular synthesis of the RNAi agent.

An RNAi-inducing entity is considered to be targeted to a target transcript for the purposes described herein if the agent comprises a strand that is substantially complementary to the target transcript over a window of evaluation between 15-29 nucleotides in length, for example, at least about 15, 17, 18, or 19 to about 21 to 23 or 24 to 29 nucleotides in length.

In some embodiments of this disclosure, the RNAi-inducing agent may contain a strand that has at least about 70%, or at least about 80%, 84%, 89%, 90%, 91%, 92%, 93%, 94%, 95%, 96%, 97%, 98%, 99%, or 100% precise sequence complementarity with the target transcript over a window of evaluation between 15-29 nucleotides in length, for example, over a window of evaluation of at least 15, more preferably at least about 17, yet more preferably at least about 18 or 19 to about 21-23 or 24-29 nucleotides in length.

The term “complementary” is used herein in accordance with its art-accepted meaning to refer to the capacity for precise pairing between particular bases, nucleosides, nucleotides or nucleic acids. For example, adenine (A) and uridine (U) are complementary; adenine (A) and thymidine (T) are complementary; and guanine (G) and cytosine (C), are complementary and are referred to in the art as Watson-Crick base pairings. If a nucleotide at a certain position of a first nucleic acid sequence is complementary to a nucleotide located opposite in a second nucleic acid sequence, the nucleotides form a complementary base pair, and the nucleic acids are complementary at that position. One of ordinary skill in the art will appreciate that the nucleic acids are aligned in antiparallel orientation (i.e, one nucleic acid is in 5′ to 3′ orientation while the other is in 3′ to 5′ orientation). A degree of complementarity of two nucleic acids or portions thereof may be evaluated by determining the total number of nucleotides in both strands that form complementary base pairs as a percentage of the total number of nucleotides over a window of evaluation when the two nucleic acids or portions thereof are aligned in antiparallel orientation for maximum complementarity.

“Administering” includes all routes of administration. Examples of routes of administration include parenteral (e.g., intravenous, intraarterial, intramuscular, subcutaneous injection), oral, inhalation, topical, nasal, and rectal.

The terms “subject,” “animal,” and “patient” include humans and other mammals, as well as cultured cells therefrom, and transgenic species thereof.

In some embodiments, the use or route of administration may be nasal spray, inhalation of liquid or powder forms, or spray or aerosol forms. In some embodiments, delivery devices include nebulizers, spray pumps, metered dose inhalers, dry powder inhalers (DPI), rotahalers, or aerosol inhalers.

In some embodiments, delivery may include intravenous injection, or a needle-free delivery device such as a powderject.

A composition or formulation to be administered will vary according to the route of administration selected (e.g., solution, emulsion, gels, aerosols, capsule). An appropriate composition comprising the compound to be administered can be prepared in a physiologically acceptable vehicle or carrier and optional adjuvants and preservatives. For solutions or emulsions, suitable carriers include, for example, aqueous or alcoholic/aqueous solutions, emulsions or suspensions, including saline and buffered media, sterile water, creams, ointments, lotions, oils, pastes and solid carriers. Parenteral vehicles can include sodium chloride solution, Ringer's dextrose, dextrose and sodium chloride, lactated Ringer's or fixed oils. Intravenous vehicles can include various additives, preservatives, or fluid, nutrient or electrolyte replenishers. See, for example, Remington's Pharmaceutical Science, 16th Edition, Mack, Ed. (1980).

For example, dosages of the active substance may be from about 0.01 mg/kg/day to about 25 mg/kg/day, advantageously from about 0.1 mg/kg/day to about 10 mg/kg/day, or from about 0.1 mg/kg/day to about 2 mg/kg/day. In some embodiments, an RNAi-inducing agent may be delivered to a subject in need thereof at a dosage of from about 0.1 mg/kg/day to about 5 mg/kg/day.

Pharmaceutically acceptable carriers include solvents, dispersion media, coatings, antibacterial and antifungal agents, isotonic and absorption delaying agents, and the like which are compatible with the activity of the compound and are physiologically acceptable to the subject.

Additional ingredients include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the disclosure are known in the art and described, for example, Remington's Pharmaceutical Science, 16th Edition, Mack, Ed. (1980).

### Viral RNAi Targets

This disclosure includes compositions and methods using RNAi for treating or preventing virus replication or infection in a subject, such as a human or non-human mammal. The virus may be an RNA virus, a negative strand virus, a positive strand virus, or a double-stranded (ds) virus.

This disclosure encompasses embodiments in which any viral gene or transcript of a viral gene that accomplishes the function of a viral protein may be an RNAi target.

For example, a target transcript may encode a protein which may be a polymerase, a nucleocapsid protein, a neuraminidase, a hemagglutinin, a matrix protein, or a nonstructural protein. In some embodiments, a target transcript may encode an influenza virus protein such as hemagglutinin, neuraminidase, membrane protein 1, membrane protein 2, nonstructural protein 1, nonstructural protein 2, polymerase protein PB1, polymerase protein PB2, polymerase protein PA, or nucleoprotein NP.

In some embodiments, a viral target RNA is the nucleoprotein or nucleocapsid transcript, or a transcript of a viral gene that accomplishes the function of the viral nucleoprotein.

Any virus containing a nucleoprotein gene or the functional equivalent thereof is suitable as an siRNA target.

Influenza nucleocapsid protein or nucleoprotein (NP) is the major structural protein that interacts with the RNA segments to form RNP. It is encoded by RNA segment 5 of influenza A virus and is 1,565 nucleotides in length. NP contains 498 amino acids. NP protein is involved in virus replication. NP-specific siRNA can inhibit the accumulation of viral RNAs in infected cells.

The ability of nucleoprotein-directed RNAi-inducing agents of this disclosure to mediate RNAi is advantageous considering the rapid mutation rate of some of the genes of some viruses, such as genes of an influenza virus. In general, the nucleoprotein gene has a lower rate of mutations as compared to other viral genes. In some embodiments, RNAi-inducing agents are targeted to conserved regions of the viral nucleoprotein gene.

Thus, the RNAi agents of this disclosure may inhibit expression of at least one target transcript involved in virus production, virus infection, virus replication, and/or transcription of viral mRNA.

### Negative Strand RNA Viruses

A viral “nucleoprotein” (also termed a “capsid protein” or a “nucleocapsid protein”) is a viral polypeptide that sequesters viral RNA and affects viral transcription. The viral nucleoprotein is capable of forming a nucleic acid/protein complex (i.e, a ribonucleoprotein (RNP) complex). Nucleoproteins are also termed “NS” in double stranded viruses (e.g., NS-6). A nucleoprotein is distinguished from an outer capsid protein, which generally does not contact and sequester the viral genome. The terms “nucleoprotein mRNA,” “NP mRNA”, “nucleoprotein transcript,” and “NP transcript” are understood to include any mRNA that encodes a viral nucleoprotein or its functional equivalent as described herein.

As will be appreciated by one of ordinary skill in the art, proteins fulfilling one or more functions of a viral nucleoprotein are referred to by a number of different names, depending on the particular virus of interest. For example, in the case of certain viruses such as influenza the protein is known as nucleoprotein (NP) while in the case of a number of other single-stranded RNA viruses, proteins that fulfill a similar role are referred to as nucleocapsid (NC or N) proteins. In yet other viruses, analogous proteins that both interact with genomic nucleic acid and play a structural role in the viral particle are considered to be capsid (C) proteins.

As used herein, the terms “nucleoprotein mRNA,” “NP mRNA”, “nucleoprotein transcript,” and “NP transcript” are understood to include any mRNA that encodes a viral nucleoprotein or its functional equivalent as described herein. Any virus containing a nucleoprotein gene or the functional equivalent thereof is suitable as a target for an RNAi-inducing agent.

Negative strand RNA viruses have a viral genome that is in the complementary sense of mRNA. Therefore, one of the first activities of negative strand RNA viruses following entry into a host cell is transcription and production of viral mRNAs. For this purpose, the virions carry an N-RNA structure that consists of the viral RNA (vRNA) that is tightly associated with the viral nucleoprotein (N or NP, sometimes called nucleocapsid protein). The RNA-dependent RNA polymerase binds either directly to the N-RNA, as is the case for influenza virus, or it binds with the help of a co-factor, like the phosphoprotein of the paramyxoviruses and the rhabdoviruses. The intact N-RNA is the actual template for transcription rather than the naked vRNA and nucleoprotein contributes to exposure of the nucleotide bases of the N-RNA for efficient reading by the polymerase.

Commonalities in expression and replication of ssRNA(−) viruses appear to include distinct transcription and replication functions for the RdRp, probably triggered by binding of the virion nucleoprotein (N or NP) subunits. Thus, both RNA(−) and RNA(+) may be found complexed with N proteins in replication complexes.

### RNA Interference

An RNAi-inducing agent can have a nucleotide length from about 10 to about 60 or more nucleotides or nucleotide analogs, or about 15-25 nucleotides or nucleotide analogs, or about 19-23 nucleotides or nucleotide analogs. The RNAi-inducing agent can have nucleotide or nucleotide analog lengths of about 10-20, 20-30, 30-40, 40-50, 15, 16, 17, 18, 19, 20, 21, 22, 23, 24, 25, 26, 27, 28, or 29 base pairs.

In some embodiments, an RNAi-inducing agent includes a 5′ terminal phosphate and/or a 3′ short overhang of about 1 or 2 nucleotides.

It is recognized that 100% sequence identity between the RNAi-inducing agent and the target gene is not required. For example, an RNAi-inducing agent having a sequence with insertions, deletions, or single point mutations relative to the target sequence, or nucleotide analog substitutions or insertions can be effective for gene silencing.

In some aspects, an RNAi-inducing agent may be designed as described in Technical Bulletin #3, Revision B, “siRNA Oligonucleotides for RNAi Applications,” Technical Bulletin #4, and “RNAi Technical Reference & Application Guide,” by Dharmacon Research Inc. (Lafayette, Colo.). Additional design considerations are described in Semizarov, D., et al., Proc. Natl. Acad. Sci. 100(11):6347-6352.

In some embodiments, greater than 70% or 80% sequence identity, for example, 84%, 89%, 91%, 92%, 93%, 94%, 95%, 96%, 97%, 98%, 99% or even 100% sequence identity, between the siRNA (e.g., the antisense strand of the siRNA) and the portion of the target gene is suitable. In the context of an siRNA of about 19-25 nucleotides, for example, at least 16-21 identical nucleotides are preferred, more preferably at least 17-22 identical nucleotides, and even more preferably at least 18-23 or 19-24 identical nucleotides. Alternatively worded, in an siRNA of about 19-25 nucleotides in length, siRNAs having no greater than about 4 mismatches are preferred, preferably no greater than 3 mismatches, more preferably no greater than 2 mismatches, and even more preferably no greater than 1 mismatch. For example, the siRNA contains an antisense strand having 1, 2, 3 or 4 mismatches with the target sequence.

In some embodiments, the RNAi molecules of this disclosure are modified, such as to improve stability in serum or in growth medium for cell cultures. In order to enhance the stability, the 3′-residues may be stabilized against degradation, for example, they may be selected such that they consist of purine nucleotides, for example, adenosine or guanosine nucleotides. Alternatively, substitution of pyrimidine nucleotides by modified analogues, for example., substitution of uridine by 2′-deoxythymidine is tolerated and does not affect the efficiency of RNA interference. For example, the absence of a 2′ hydroxyl may significantly enhance the nuclease resistance of the siRNAs in tissue culture medium.

In some embodiments, the RNAi agent may contain at least one modified nucleotide analogue. The nucleotide analogue may be located at a position where the target-specific activity, for example, the RNAi mediating activity is not substantially affected, for example, in a region at the 5′-end and/or the 3′-end of the RNA molecule. In particular, the ends may be stabilized by incorporating a modified nucleotide analogue. Such nucleotide analogues include sugar- and/or backbone-modified ribonucleotides. For example, the phosphodiester linkages of natural RNA may be modified to include a nitrogen or sulfur heteroatom. In some backbone-modified ribonucleotides the phosphoester group connecting adjacent ribonucleotides may be replaced by a modified group, for example, a phosphorothioate group. In some sugar-modified ribonucleotides, the 2′-OH group may be replaced by a group selected from H, OR, R, halo, SH, SR, NH₂, NHR, NR₂ or ON, wherein R is C₁-C₆ alkyl, alkenyl, or alkynyl, and halo is F, Cl, Br or I.

Other nucleobase-modified ribonucleotides include ribonucleotides containing at least one non-naturally occurring nucleobase instead of a naturally occurring nucleobase. Bases may be modified to block the activity of adenosine deaminase. Exemplary modified nucleobases include uridine and/or cytidine modified at the 5-position, for example, 5-(2-amino)propyl uridine, 5-bromo uridine; adenosine and/or guanosines modified at the 8 position, for example, 8-bromo guanosine; deaza nucleotides, for example, 7-deaza-adenosine; O- and N-alkylated nucleotides, for example, N6-methyl adenosine, or a combination thereof.

In some embodiments, an RNAi-agent can be modified by the substitution of at least one nucleotide with a modified nucleotide. The RNAi-agent can have one or more mismatches when compared to the target sequence of the nucleoprotein transcript and still mediate RNAi as demonstrated in the examples below.

An RNAi-agent may be synthesized either in vivo or in vitro. Endogenous RNA polymerase of the cell may mediate transcription in vivo, or cloned RNA polymerase can be used for transcription in vivo or in vitro. For transcription from a transgene in vivo or an expression construct, a regulatory region (e.g., promoter, enhancer, silencer, or splice donor and acceptor) may be used to transcribe the RNAi-agent. Inhibition may be targeted by specific transcription in an organ, tissue, or cell type; stimulation of an environmental condition (e.g., infection, stress, temperature, chemical inducers); and/or engineering transcription at a developmental stage or age. A transgenic organism that expresses an RNAi-agent from a recombinant construct may be produced by introducing the construct into a zygote, an embryonic stem cell, or another multipotent cell derived from the appropriate organism.

RNA may be produced enzymatically or by partial/total organic synthesis, any modified ribonucleotide can be introduced by in vitro enzymatic or organic synthesis. In some embodiments, a siRNA is prepared chemically. Methods of synthesizing RNA molecules are known in the art, in particular, the chemical synthesis methods as de scribed in Verma and Eckstein, Annul Rev. Biochem. 67:99-134 (1998). In some embodiments, an RNAi-agent is prepared enzymatically. For example, an RNAi-agent can be prepared by enzymatic processing of a long dsRNA having sufficient complementarity to the desired target RNA. Processing of long dsRNA can be accomplished in vitro, for example, using appropriate cellular lysates and ds-siRNAs can be subsequently purified by gel electrophoresis or gel filtration. In an exemplary embodiment, RNA can be purified from a mixture by extraction with a solvent or resin, precipitation, electrophoresis, chromatography, or a combination thereof. Alternatively, the RNA may be used with no or a minimum of purification to avoid losses due to sample processing.

An RNAi-agent can also be prepared by enzymatic transcription from synthetic DNA templates or from DNA plasmids isolated from recombinant bacteria. Typically, phage RNA polymerases are used such as T7, T3 or SP6 RNA polymerase (Milligan & Uhlenbeck, Methods Enzymol. 180:51-62 (1989)). The RNA may be dried for storage or dissolved in an aqueous solution. The solution may contain buffers or salts to inhibit annealing, and/or promote stabilization of the single strands.

An RNAi-agent may be derived from a vector that expresses one or more RNAi-agents that include sequences sufficiently complementary to a portion of the target gene to mediate RNAi. The vector can be administered in vivo to thereby initiate RNAi therapeutically or prophylactically by expression of one or more copies of the RNAi-agents. In one embodiment, synthetic shRNA is expressed in a plasmid vector. In another, the plasmid is replicated in vivo. In another embodiment, the vector can be a viral vector, for example, a retroviral vector.

Some target viruses mutate rapidly and may result in a mismatch of even one nucleotide that can, in some instances, impede RNAi. Accordingly, in one embodiment, a vector is contemplated that expresses a plurality of RNAi-agents to increase the probability of sufficient homology to mediate RNAi. These RNAi-agents may be staggered along the nucleoprotein gene, or clustered in one region of the nucleoprotein gene. For example, a plurality of RNAi-agents may be directed towards a region of the nucleoprotein gene that is about 200 nucleotides in length and contains the 3′ end of the nucleoprotein gene.

This disclosure encompasses methods for diagnosing virus infection and for determining whether a subject is infected with a virus. In some embodiments, it may be determined whether a subject is infected with a virus that can be inhibited by one or more RNAi-inducing entities. For example, a sample (e.g., sputum, saliva, nasal washings, nasal swab, throat swab, bronchial washings, broncheal alveolar lavage (BAL) fluid, biopsy specimens, etc.) may be obtained from a subject who may be suspected of having a viral infection, for example, influenza, and may be analyzed to determine whether it contains a virus-specific nucleic acid. Some assays for detection and/or genotyping of infectious agents are described in Molecular Microbiology: Diagnostic Principles and Practice, Persing, D. H., et al., (eds.) Washington, D.C.: ASM Press, 2004.

### Uses of RNAi

This disclosure encompasses both prophylactic and therapeutic methods for treating a subject at risk of, or susceptible to, or having a virus or viral infection. The use of RNAi agents includes uses for preparation of medicaments for healing, alleviating, relieving, altering, remedying, or ameliorating symptoms or conditions caused by the virus or viral infection.

In some aspects, this disclosure provides uses for preventing in a subject, infection by a virus, or a condition associated with a viral infection, by administering to the subject a prophylactically- or therapeutically-effective RNAi agent. The use of an RNAi agent can occur prior to the manifestation of symptoms characteristic of a viral infection, or post-infection, so that the viral infection is prevented or ameliorated.

As used herein, “therapeutically effective”, “efficacy”, “therapeutic efficacy”, “therapeutic methods” as it relates to RNAi-inducing agents, methods, and uses thereof refers to a delay or inhibition of virus progression, reduction in viral titer, prevention or inhibition of viral replication, reduction of viral RNA levels, prevention of viral RNA expression, prevention or reduction of systemic circulation of influenza virus, prevention or reduction of virus-related complications, reduction or prevention of virus-induced illness or symptoms in a subject.

In some embodiments, the RNAi agent may be administered to the subject prior to exposure to the target virus.

In some embodiments, the RNAi agent may be administered to the subject after exposure to the target virus to delay or inhibit its progression, or prevent its integration into healthy cells or cells that do not contain virus.

In some embodiments, the RNAi agent may be administered to the subject after exposure to the target virus to reduce viral titers in the subject.

In some embodiments, target virus formation is inhibited or prevented. In some embodiments, target virus replication is inhibited or prevented.

In some embodiments, the RNAi agent may be administered to the subject before or after exposure to the target virus to reduce viral RNA levels or prevent viral RNA expression.

The therapeutic methods of this disclosure are capable of reducing viral production in a subject, for example, viral titer or provirus titer, by at least about 2 to about 10-fold, about 10 to about 50-fold, about 60 to about 80-fold, or at least 100-fold, 200-fold, 300-fold, or greater.

The therapeutic methods of this disclosure are capable of reducing viral production in a subject, for example, viral titer or provirus titer, by at least about 10%, 20%, 30%, 40%, 50%, 60%, 70%, 80%, 90% or more.

In some embodiments, the therapeutic methods of this disclosure are capable of reducing the duration of influenza infection or illness by about 1, 2, 3, 4, 5, or more days.

In some embodiments, the therapeutic methods of this disclosure are capable of reducing the severity of influenza infection or illness.

In some embodiments, the therapeutic methods of this disclosure are capable of preventing or reducing the systemic circulation levels of influenza virus in a subject. In some aspects, the therapeutic methods of this disclosure reduce the systemic circulation levels of influenza virus, by at least about 10%, 20%, 30%, 40%, 50%, 60%, 70%, 80%, 90% or more.

In some embodiments, the therapeutic methods of this disclosure are capable of preventing or reducing serious influenza-related complications (e.g., bacterial, or viral pneumonia or exacerbation of chronic diseases) in children, adults, elderly, or otherwise subjects with compromised immune systems.

In some embodiments, a subject that has been exposed to the target virus can be treated both prophylactically and therapeutically.

The RNAi agents of this disclosure can be used in combination with other therapeutic components, for example, other anti-viral drugs or therapeutics. Examples of therapeutic components that can be used in conjunction with RNAi therapy include antiviral compounds, immunomodulators, immunostimulants, and antibiotics that can be employed to treat viral infections. Immunomodulators and immunostimulants include, for example, various interleukins, CD4, cytokines, antibody preparations, blood transfusions, and cell transfusions.

### Pharmaceutical Compositions

The RNAi agents of this disclosure can be incorporated into pharmaceutical compositions suitable for administration. Such compositions may include the agent and a carrier.

A pharmaceutical composition of the disclosure is formulated to be compatible with its intended route of administration. Examples of routes of administration include oral, by inhalation, intranasal, parenteral (e.g., intravenous, intradermal, subcutaneous, intraperitoneal, and intramuscular), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates and agents for the adjustment of tonicity such as sodium chloride or dextrose. The pH of a composition or formulation can be adjusted with acids or bases, such as hydrochloric acid or sodium hydroxide. The parenteral preparation can be enclosed in ampoules, disposable syringes or multiple dose vials made of glass or plastic.

For intranasal administration, an RNAi-inducing agent dose may from about 0.01 mg/mL to about 20 mg/mL, or about 0.5 mg/mL to about 15 mg/mL or about 1 mg/mL to about 10 mg/mL with up to about 1, 2, 3, 4, 5, 6, 7, or 8 100 μL sprays/nostril. For example, this would encompass a 0.06 mg/kg dose in a 70 kg human. An RNAi-inducing agent may be administered to a subject in a dose of from about 1 mg/mL to about 100 mg/mL solution with up to about 1, 2, 3, 4, 5, 6, 7, or 8 100 μL sprays/nostril. An RNAi-inducing agent may be administered to a subject in a dose of from about 0.00005 mg/kg to about 5 mg/kg, or about 0.001 mg/kg to about 2 mg/kg, or about 0.006 mg/kg to about 0.6 mg/kg. The dosing regimen may be 8, 7, 6, 5, 4, 3, 2, or 1 time per day.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid for syringability.

The carrier can be a solvent or dispersion medium containing, for example, water, ethanol, polyol (e.g., glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents (e.g., parabens, chlorobutanol, benzalkonium chloride, phenol, ascorbic acid, thimerosal, and the like). In some embodiments, isotonic agents (e.g., sugars, polyalcohols such as mannitol, sorbitol, and sodium chloride) are included in the composition.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation include vacuum drying and freeze-drying which yields a powder of the active ingredient plus any additional desired ingredient from a previously sterile-filtered solution thereof.

Inhalational administration means the RNAi-inducing entity is introduced directly to the respiratory system by inhalation through the nose or mouth and into the lungs. The entity may be in naked form or with a delivery agent. In some embodiments, the RNAi-inducing agent may be administered in an amount effective to treat or prevent a condition that affects the respiratory system, such as a respiratory virus infection, while resulting in minimal absorption into the blood and thus minimal systemic delivery of the RNAi-inducing agent.

For inhalation administration, an RNAi-inducing agent dose may from about 0.01 mg/mL to about 20 mg/mL, or about 0.5 mg/mL to about 15 mg/mL or about 1 mg/mL to about 10 mg/mL. For example, this would encompass a 0.06 mg/kg dose in a 70 kg human. An RNAi-inducing agent may be administered to a subject in a dose of from about 1 mg/mL to about 100 mg/mL. An RNAi-inducing agent may be administered to a subject in a dose of from about 0.00005 mg/kg to about 5 mg/kg, or about 0.001 mg/kg to about 5 mg/kg, or about 1 mg/kg to about 4 mg/kg, The dosing regimen may be 8, 7, 6, 5, 4, 3, 2, or 1 time per day.

In particular, dry powder compositions containing RNAi-inducing entities may be delivered in the form of an aerosol spray from a pressured container or dispenser which contains a suitable propellant, for example, a gas such as carbon dioxide, or a nebulizer. In some embodiments, the delivery system can be suitable for delivering the composition into major airways (trachea and bronchi) of a subject, and/or deeper into the lung (bronchioles and/or alveoli). An RNAi-inducing entity may be delivered as a nasal spray.

For example, RNAi-inducing agents can be delivered to the lungs as a composition of the RNAi-inducing agent in dry form (e.g., dry powder) or in an aqueous medium, optionally including a salt (e.g., NaCl, a phosphate salt), buffer, and/or an alcohol.

Oral compositions may include an inert diluent or an edible carrier. They can be enclosed in gelatin capsules or compressed into tablets. For the purpose of oral therapeutic administration, the active compound can be incorporated with excipients and used in the form of so tablets, troches, or capsules. Oral compositions can also be prepared using a fluid carrier for use as a wash.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

In some embodiments, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid.

Inhalational, oral or parenteral compositions may be formulated in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

All publications, references, patents, patent publications and patent applications cited herein are each hereby specifically incorporated by reference in its entirety.

While this disclosure has been described in relation to certain embodiments, and many details have been set forth for purposes of illustration, it will be apparent to those skilled in the art that this disclosure includes additional embodiments, and that some of the details described herein may be varied considerably without departing from this disclosure. This disclosure includes such additional embodiments, modifications and equivalents. In particular, this disclosure includes any combination of the features, terms, or elements of the various illustrative components and examples provided herein.

The use herein of the terms “a,” “an,” “the,” and similar terms in describing the disclosure, and in the claims, are to be construed to include both the singular and the plural. The terms “comprising,” “having,” “including,” and “containing” are to be construed as open-ended terms which mean, for example, “including, but not limited to.” Recitation of a range of values herein refers individually to each and any separate value falling within the range as if it were individually recited herein, whether or not some of the values within the range are expressly recited. For example, the range “4 to 12” includes without limitation the values 5, 5.1, 5.35 and any other whole or fractional value greater than or equal to 4 and less than or equal to 12. Specific values employed herein will be understood as exemplary and not to limit the scope of the disclosure.

Definitions of technical terms provided herein should be construed to include without recitation those meanings associated with those terms as known to those skilled in the art, and are not intended to limit the scope of the disclosure. Definitions of technical terms provided herein shall be construed to dominate over alternative definitions in the art or definitions which become incorporated herein by reference to the extent that the alternative definitions conflict with the definition provided herein.

The examples given herein, and the exemplary language used herein are solely for the purpose of illustration, and are not intended to limit the scope of the disclosure.

When a list of examples is given, such as a list of compounds or molecules suitable for this disclosure, it will be apparent to those skilled in the art that mixtures of the listed compounds or molecules are also suitable.

### EXAMPLE 1

### Identification of Viral Nucleoproteins and siRNAs

Highly conserved sites are considered to be those sites or sequences that are found to be present in a high proportion of all the published human influenza sequences. A subsidiary goal was to identify 19-mer and 25-mer sequences in human influenza isolates that are similar to the highly conserved 19-mer and 25-mer sequences, but that differ by only one or a few nucleotide changes.

There are eight separate RNA segments that compose the influenza viral genome. All analyses were done separately for each of the viral segments. Thus, for example, a search for conserved sites was performed for viral segment #1 using only sequences obtained from segment #1.

Influenza A viral sequences from each of the eight viral segments was obtained from the Influenza Sequence Database (Macken, C., Lu, H., Goodman, J., & Boykin, L., “The value of a database in surveillance and vaccine selection.” in Options for the Control of Influenza IV. A.D.M.E. Osterhaus, N. Cox & A. W. Hampson (Eds.) Amsterdam: Elsevier Science, 2001, 103-106), abbreviated in this document as ISD. The list was screened to remove all but nearly full-length sequences (those with a length that is at least 90% of the longest observed length for a given segment), so that a failure to find a 19-mer or 25-mer fragment match within a given target sequence could not be ascribed to sequence truncation.

### EXAMPLE 2

### Synthesized siRNAs

Tables 1 and 2 list the influenza dsRNAs that were synthesized. Table 1 lists the sequences of the sense strands, while Table 2 lists the corresponding sequences of the antisense strands, in order of appearance.

The sequences listed in Table 1 are numbered SEQ ID NOS:1-58, respectively in order of appearance.

TABLE 1

Sense Strands of Influenza dsRNAs

| | | SEQ |
|---|---|---|
| | | ID |
| Compound | Sense Sequence | NO: |
| DX2844 | Cy5; rG; rG; rA; rU; rC; rU; rU; rA; rU; rU; rU; rC; rU; rU; rC; rG; rG; rA; rG; dT; dT | 1 |
| DX3003 | Cy5; rG; rG; rA; rU; rC; rU; rU; rA; rU; rU; rU; rC; rU; rU; rC; rG; rG; rA; rG; dT; dT | 2 |
| DX2852 | rA; rG; rA; rC; rA; rG; rC; rG; rA; rC; rC; rA; rA; rA; rA; rG; rA; rA; rU; rU; rC; rG; rG; | 3 |
| | dA; dT | |
| DX3044 | rA; rG; rA; rC; rA; rG; rC; rG; rA; rC; rC; rA; rA; rA; rA; rG; rA; rA; rU; rU; rC; rG; rG; | 4 |
| | dA; dT | |
| DX2855 | rA; rU; rG; rA; rA; rG; rA; rU; rC; rU; rG; rU; rU; rC; rC; rA; rC; rC; rA; rU; rU; rG; rA; | 5 |
| | dA; dG | |
| DX3046 | rA; rU; rG; rA; rA; rG; rA; rU; rC; rU; rG; rU; rU; rC; rC; rA; rC; rC; rA; rU; rU; rG; rA; | 6 |
| | dA; dG | |
| DX2858 | rG; rA; rU; rC; rU; rG; rU; rU; rC; rC; rA; rC; rC; rA; rU; rU; rG; rA; rA; rG; rA; rA; rC; | 7 |
| | dT; dC | |
| DX3048 | rG; rA; rU; rC; rU; rG; rU; rU; rC; rC; rA; rC; rC; rA; rU; rU; rG; rA; rA; rG; rA; rA; rC; | 8 |
| | dT; dC | |
| DX2861 | rU; rU; rG; rA; rG; rG; rA; rG; rU; rG; rC; rC; rU; rG; rA; rU; rU; rA; rA; rU; rG; rA; rU; | 9 |
| | dC; dC | |
| DX3050 | rU; rU; rG; rA; rG; rG; rA; rG; rU; rG; rC; rC; rU; rG; rA; rU; rU; rA; rA; rU; rG; rA; rU; | 10 |
| | dC; dC | |
| DX2871 | rG; rG; rC; rU; rC; rU; rU; rA; rU; rU; rU; rC; rU; rU; rC; rG; rG; rA; rG; dT; dT | 11 |
| DX2874 | rG; rG; rA; rU; rC; rC; rU; rA; rU; rU; rU; rC; rU; rU; rC; rG; rG; rA; rG; dT; dT | 12 |
| DX2877 | rG; rG; rA; rU; rC; rU; rU; rA; rC; rU; rU; rC; rU; rU; rC; rG; rG; rA; rG; dT; dT | 13 |
| DX2744 | rG; rG; rA; rU; rC; rU; rU; rA; rU; rU; rU; rC; rU; rU; rC; rG; rG; rA; rG; dT; dT | 14 |
| DX2880 | rG; rG; rA; rU; rC; rU; rU; rA; rU; rU; rU; rC; rU; rU; rC; rG; rG; rA; rG; dT; dT | 15 |
| DX2882 | rG; rG; rA; rU; rC; rU; rU; rA; rU; rU; rU; rC; rU; rU; rC; rG; rG; rA; rG; dT; dT | 16 |
| DX2889 | rG; rG; rA; rU; rC; rU; rU; rA; rU; rU; rU; rC; rU; rU; rC; rG; rG; rA; rG; dT; dT | 17 |
| DX2890 | rG; rG; rA; rU; rC; rU; rU; rA; rU; rU; rU; rC; rU; rU; rC; rG; rG; rA; rG; dT; dT | 18 |
| DX2891 | rG; rG; rA; rU; rC; rU; rU; rA; rU; rU; rU; rC; rU; rU; rC; rG; rG; rA; rG; dT; dT | 19 |
| DX2892 | rG; rG; rA; rU; rC; rU; rU; rA; rU; rU; rU; rC; rU; rU; rC; rG; rG; rA; rG; dT; dT | 20 |
| DX2888 | rG; rG; rA; rU; rC; rU; rU; rA; rU; rU; rU; rC; rU; rU; rC; rG; rG; rC; rG; dT; dT | 21 |
| DX2895 | rG; rA; rU; rC; rU; rU; rA; rU; rU; rU; rC; rU; rU; rC; rG; rG; rA; rG; rA; dT; dT | 22 |
| DX2906 | rG; rA; rU; rC; rU; rU; rA; rU; rU; rU; rC; rU; rU; rC; rG; rG; rA; rG; rA; dT; dT | 23 |
| DX2908 | rG; rA; rU; rC; rU; rU; rA; rU; rU; rU; rC; rU; rU; rC; rG; rG; rA; rG; rA; dT; dT | 24 |
| DX2912 | rG; rA; rU; rC; rU; rU; rA; rU; rU; rU; rC; rU; rU; rC; rG; rG; rA; rG; rA; dT; dT | 25 |
| DX2913 | rG; rA; rU; rC; rU; rU; rA; rU; rU; rU; rC; rU; rU; rC; rG; rG; rA; rG; rA; dT; dT | 26 |
| DX2914 | rG; rA; rU; rC; rU; rU; rA; rU; rU; rU; rC; rU; rU; rC; rG; rG; rA; rG; rA; dT; dT | 27 |
| DX2915 | rG; rA; rU; rC; rU; rU; rA; rU; rU; rU; rC; rU; rU; rC; rG; rG; rA; rG; rA; dT; dT | 28 |
| DX2898 | rG; rC; rU; rC; rU; rU; rA; rU; rU; rU; rC; rU; rU; rC; rG; rG; rA; rG; rA; dT; dT | 29 |
| DX2901 | rG; rA; rU; rC; rC; rU; rA; rU; rU; rU; rC; rU; rU; rC; rG; rG; rA; rG; rA; dT; dT | 30 |
| DX2904 | rG; rA; rU; rC; rU; rU; rA; rC; rU; rU; rC; rU; rU; rC; rG; rG; rA; rG; rA; dT; dT | 31 |
| DX2911 | rG; rA; rU; rC; rU; rU; rA; rU; rU; rU; rC; rU; rU; rC; rG; rG; rC; rG; rA; dT; dT | 32 |
| DX3054 | rG; rA; rU; rC; rU; rU; rA; rU; rU; rU; rC; rU; rU; rC; rG; rG; rA; rG; rA; rC; rA; rA; dT; | 33 |
| | dG | |
| DX3056 | rA; rU; rC; rU; rU; rA; rU; rU; rU; rC; rU; rU; rC; rG; rG; rA; rG; rA; rC; rA; rA; dT; dG | 34 |
| DX2956 | rG; rG; rA; rU; rC; rU; rU; rA; rU; rU; rU; rC; rU; rU; rC; rG; rG; rA; rG; rA; rC; rA; rA; | 35 |
| | dT; dG | |
| DX3030 | rG; rG; rA; rU; rC; rU; rU; rA; rU; rU; rU; rC; rU; rU; rC; rG; rG; rA; rG; rA; rC; rA; rA; | 36 |
| | dT; dG | |
| DX3052 | rG; rG; rA; rU; rC; rU; rU; rA; rU; rU; rU; rC; rU; rU; rC; rG; rG; rA; rG; rA; rC; rA; rA; | 37 |
| | dT; dG | |
| DX3058 | rG; rG; rA; rU; rC; rU; rU; rA; rU; rU; rU; rC; rU; rU; rC; rG; rG; rA; rG; rA; rC; rA; rA; | 38 |
| | dT; dG | |
| DX3060 | rG; rG; rA; rU; rC; rU; rU; rA; rU; rU; rU; rC; rU; rU; rC; rG; rG; rA; rG; rA; rC; rA; rA; | 39 |
| | dT; dG | |
| DX3161 | rG; rG; rA; rU; rC; rU; rU; rA; rU; rU; rU; rC; rU; rU; rC; rG; rG; rA; rG; rA; rC; rA; rA; | 40 |
| | dT; dG | |
| DX2819 | rG; rA; rU; rC; rU; rG; rU; rU; rC; rC; rA; rC; rC; rA; rU; rU; rG; rA; rA; dT; dT | 41 |
| DX2820 | rA; rU; rG; rA; rA; rG; rA; rU; rC; rU; rG; rU; rU; rC; rC; rA; rC; rC; rA; dT; dT | 42 |
| DX2821 | rG; rC; rA; rA; rU; rU; rG; rA; rG; rG; rA; rG; rU; rG; rC; rC; rU; rG; rA; dT; dT | 43 |
| DX2822 | rU; rU; rG; rA; rG; rG; rA; rG; rU; rG; rC; rC; rU; rG; rA; rU; rU; rA; rA; dT; dT | 44 |
| DX2823 | rC; rG; rG; rG; rA; rC; rU; rC; rU; rA; rG; rC; rA; rU; rA; rC; rU; rU; rA; dT; dT | 45 |
| DX2824 | rA; rC; rU; rG; rA; rC; rA; rG; rC; rC; rA; rG; rA; rC; rA; rG; rC; rG; rA; dT; dT | 46 |
| DX2825 | rA; rG; rA; rC; rA; rG; rC; rG; rA; rC; rC; rA; rA; rA; rA; rG; rA; rA; rU; dT; dT | 47 |
| DX2962 | rG; rG; rA; rT; rC; rT; rT; rA; rT; rT; rT; rC; rT; rT; rC; rG; rG; rA; rG; dT; dT | 48 |
| DX3078 | Cy5; rG; rG; rA; rU; rC; rU; rU; rA; rU; rU; rU; rC; rU; rU; rC; rG; rG; rA; rG; rA; rC; | 49 |
| | rA; rA; dT; dG | |
| DX3151 | rG; rG; rA; rU; rC; rU; rU; rA; rU; rU; rU; rC; rU; rU; rC; rG; rG; rA; rG; rA; rC; rA; rA; | 50 |
| | rT; dG | |
| DX3154 | rG; rG; rA; rU; rC; rU; rU; rA; rU; rU; rU; rC; rU; rU; rC; rG; rG; rA; rG; rA; rC; rA; rA; | 51 |
| | rT; rT | |
| DX3156 | rG; rG; rA; rT; rC; rT; rT; rA; rT; rT; rT; rC; rT; rT; rC; rG; rG; rA; rG; rA; rC; rA; rA; | 52 |
| | rT; dG | |
| DX3159 | rG; omeG; omeA; rU; rC; rU; rU; rA; rU; rU; rU; rC; rU; rU; rC; rG; rG; rA; rG; rA; rC; rA; | 53 |
| | rA; dT; dG | |
| DX3160 | rG; omeG; omeA; rU; rC; rU; rU; rA; rU; rU; rU; rC; rU; rU; rC; rG; rG; rA; rG; rA; rC; rA; | 54 |
| | rA; dT; dG | |
| DX3163 | rG; rG; rA; rU; rC; rU; rU; rA; rU; rU; rU; rC; rU; rU; rC; rG; rG; rA; rG; rA; rC; rA; rA; | 55 |
| | omeU; omeG | |
| DX3165 | rG; rG; rA; rU; rC; rU; rU; rA; rU; rU; rU; rC; rU; rU; rC; rG; rG; rA; rG; rA; rC; omeA; | 56 |
| | omeA; dT; dG | |
| DX3029 | rG; rG; rA; rT; rC; rT; rT; rA; rT; rT; rT; rC; rT; rT; rC; rG; rG; rA; rG; rA; rC; rA; rA; | 57 |
| | dT; dG | |
| DX3076 | rG; rG; rA; rT; rC; rT; rT; rA; rT; rT; rT; rC; rT; rT; rC; rG; rG; rA; rG; rA; rC; rA; rA; | 58 |
| | dT; dG | |

The sequences listed in Table 2 are numbered SEQ ID NOS:59-116, respectively in order of appearance.

TABLE 2

Antisense Strands of Influenza siRNAs

| | SEQ |
|---|---|
| | ID |
| Antisense Sequence | NO: |
| p; rC; rU; rC; rC; rG; rA; rA; rG; rA; rA; rA; rU; rA; rA; rG; rA; rC; rC; rU; dT; dT | 59 |
| rC; rU; rC; rC; rG; rA; rA; rG; rA; rA; rA; rU; rA; rA; rG; rA; rU; rC; rC; dT; dT | 60 |
| rA; rU; rC; rC; rG; rA; rA; rU; rU; rC; rU; rU; rU; rU; rG; rG; rU; rC; rG; rC; rU; rG; rU; rC; rU; | 61 |
| dT; dT | |
| rA; rU; rC; rC; rG; rA; rA; rU; rU; rC; rU; rU; rU; rU; rG; rG; rU; rC; rG; rC; rU; rG; rU; rC; rU; | 62 |
| rU; rU | |
| rC; rU; rU; rC; rA; rA; rU; rG; rG; rU; rG; rG; rA; rA; rC; rA; rG; rA; rU; rC; rU; rU; rC; rA; rU; | 63 |
| dT; dT | |
| rC; rU; rU; rC; rA; rA; rU; rG; rG; rU; rG; rG; rA; rA; rC; rA; rG; rA; rU; rC; rU; rU; rC; rA; rU; | 64 |
| rU; rU | |
| rG; rA; rG; rU; rU; rC; rU; rU; rC; rA; rA; rU; rG; rG; rU; rG; rG; rA; rA; rC; rA; rG; rA; rU; rC; | 65 |
| dT; dT | |
| rG; rA; rG; rU; rU; rC; rU; rU; rC; rA; rA; rU; rG; rG; rU; rG; rG; rA; rA; rC; rA; rG; rA; rU; rC; | 66 |
| rU; rU | |
| rG; rG; rA; rU; rC; rA; rU; rU; rA; rA; rU; rC; rA; rG; rG; rC; rA; rC; rU; rC; rC; rU; rC; rA; rA; | 67 |
| dT; dT | |
| rG; rG; rA; rU; rC; rA; rU; rU; rA; rA; rU; rC; rA; rG; rG; rC; rA; rC; rU; rC; rC; rU; rC; rA; rA; | 68 |
| rU; rU | |
| rC; rU; rC; rC; rG; rA; rA; rG; rA; rA; rA; rU; rA; rA; rG; rA; rI; rC; rC; dT; dT | 69 |
| rC; rU; rC; rC; rG; rA; rA; rG; rA; rA; rA; rU; rA; rI; rG; rA; rU; rC; rC; dT; dT | 70 |
| rC; rU; rC; rC; rG; rA; rA; rG; rA; rA; rI; rU; rA; rA; rG; rA; rU; rC; rC; dT; dT | 71 |
| rC; rU; rC; rC; rG; rA; rA; rG; rA; rA; rA; rU; rA; rA; rG; rA; rU; rC; rC; dT; dT | 72 |
| rC; rU; rC; rC; rG; rA; rA; rI; rA; rA; rA; rU; rA; rA; rG; rA; rU; rC; rC; dT; dT | 73 |
| rC; rU; rC; rC; rI; rA; rA; rG; rA; rA; rA; rU; rA; rA; rG; rA; rU; rC; rC; dT; dT | 74 |
| rC; rU; rC; rC; rG; rA; rA; rG; rA; rA; rA; rU; rA; rA; rG; rA; rI; rC; rC; dT; dT | 75 |
| rC; rU; rC; rC; rG; rA; rA; rG; rA; rA; rA; rU; rA; rI; rG; rA; rU; rC; rC; dT; dT | 76 |
| rC; rU; rC; rC; rG; rA; rA; rG; rA; rA; rI; rU; rA; rA; rG; rA; rU; rC; rC; dT; dT | 77 |
| rC; rI; rC; rC; rG; rA; rA; rG; rA; rA; rA; rU; rA; rA; rG; rA; rU; rC; rC; dT; dT | 78 |
| rC; rI; rC; rC; rG; rA; rA; rG; rA; rA; rA; rU; rA; rA; rG; rA; rU; rC; rC; dT; dT | 79 |
| rU; rC; rU; rC; rC; rG; rA; rA; rG; rA; rA; rA; rU; rA; rA; rG; rA; rU; rC; dT; dT | 80 |
| rU; rC; rU; rC; rC; rG; rA; rA; rI; rA; rA; rA; rU; rA; rA; rG; rA; rU; rC; dT; dT | 81 |
| rU; rC; rU; rC; rC; rI; rA; rA; rG; rA; rA; rA; rU; rA; rA; rG; rA; rU; rC; dT; dT | 82 |
| rU; rC; rU; rC; rC; rG; rA; rA; rG; rA; rA; rA; rU; rA; rA; rG; rA; rI; rC; dT; dT | 83 |
| rU; rC; rU; rC; rC; rG; rA; rA; rG; rA; rA; rA; rU; rA; rI; rG; rA; rU; rC; dT; dT | 84 |
| rU; rC; rU; rC; rC; rG; rA; rA; rG; rA; rA; rI; rU; rA; rA; rG; rA; rU; rC; dT; dT | 85 |
| rU; rC; rI; rC; rC; rG; rA; rA; rG; rA; rA; rA; rU; rA; rA; rG; rA; rU; rC; dT; dT | 86 |
| rU; rC; rU; rC; rC; rG; rA; rA; rG; rA; rA; rA; rU; rA; rA; rG; rA; rI; rC; dT; dT | 87 |
| rU; rC; rU; rC; rC; rG; rA; rA; rG; rA; rA; rA; rU; rA; rI; rG; rA; rU; rC; dT; dT | 88 |
| rU; rC; rU; rC; rC; rG; rA; rA; rG; rA; rA; rI; rU; rA; rA; rG; rA; rU; rC; dT; dT | 89 |
| rU; rC; rI; rC; rC; rG; rA; rA; rG; rA; rA; rA; rU; rA; rA; rG; rA; rU; rC; dT; dT | 90 |
| rC; rA; rU; rU; rG; rU; rC; rU; rC; rC; rG; rA; rA; rG; rA; rA; rA; rU; rA; rA; rG; rA; rU; rC; rC; | 91 |
| rU; rU | |
| rC; rA; rU; rU; rG; rU; rC; rU; rC; rC; rG; rA; rA; rG; rA; rA; rA; rU; rA; rA; rG; rA; rU; rC; rC; | 92 |
| rU; rU | |
| rC; rA; rU; rU; rG; rU; rC; rU; rC; rC; rG; rA; rA; rG; rA; rA; rA; rU; rA; rA; rG; rA; rU; rC; rC; | 93 |
| dT; dT | |
| rC; rA; rU; rU; rG; rU; rC; rU; rC; rC; rG; rA; rA; rG; rA; rA; rA; rU; rA; rA; rG; rA; rU; rC; rC; | 94 |
| rU; rU | |
| rC; rA; rU; rU; rG; rU; rC; rU; rC; rC; rG; rA; rA; rG; rA; rA; rA; rU; rA; rA; rG; rA; rU; rC; rC; | 95 |
| rT; rT | |
| rA; rU; rU; rG; rU; rC; rU; rC; rC; rG; rA; rA; rG; rA; rA; rA; rU; rA; rA; rG; rA; rU; rC; rC; rU; | 96 |
| rU | |
| rU; rU; rG; rU; rC; rU; rC; rC; rG; rA; rA; rG; rA; rA; rA; rU; rA; rA; rG; rA; rU; rC; rC; rU; rU | 97 |
| rC; rA; rU; rU; rG; rU; rC; rU; rC; rC; rG; rA; rA; rG; rA; rA; rA; rU; rA; rA; rG; rA; rU; omeC; | 98 |
| omeC; rU; rU | |
| rU; rU; rC; rA; rA; rU; rG; rG; rU; rG; rG; rA; rA; rC; rA; rG; rA; rU; rC; dT; dT | 99 |
| rU; rG; rG; rU; rG; rG; rA; rA; rC; rA; rG; rA; rU; rC; rU; rU; rC; rA; rU; dT; dT | 100 |
| rU; rC; rA; rG; rG; rC; rA; rC; rU; rC; rC; rU; rC; rA; rA; rU; rU; rG; rC; dT; dT | 101 |
| rU; rU; rA; rA; rU; rC; rA; rG; rG; rC; rA; rC; rU; rC; rC; rU; rC; rA; rA; dT; dT | 102 |
| rU; rA; rA; rG; rU; rA; rU; rG; rC; rU; rA; rG; rA; rG; rU; rC; rC; rC; rG; dT; dT | 103 |
| rU; rC; rG; rC; rU; rG; rU; rC; rU; rG; rG; rC; rU; rG; rU; rC; rA; rG; rU; dT; dT | 104 |
| rA; rU; rU; rC; rU; rU; rU; rU; rG; rG; rU; rC; rG; rC; rU; rG; rU; rC; rU; dT; dT | 105 |
| rC; rT; rC; rC; rG; rA; rA; rG; rA; rA; rA; rT; rA; rA; rG; rA; rT; rC; rC; dT; dT | 106 |
| rC; rA; rU; rU; rG; rU; rC; rU; rC; rC; rG; rA; rA; rG; rA; rA; rA; rU; rA; rA; rG; rA; rU; rC; rC; | 107 |
| rU; rU | |
| rC; rA; rU; rU; rG; rU; rC; rU; rC; rC; rG; rA; rA; rG; rA; rA; rA; rU; rA; rA; rG; rA; rU; rC; rC; | 108 |
| rU; rU | |
| rA; rA; rU; rU; rG; rU; rC; rU; rC; rC; rG; rA; rA; rG; rA; rA; rA; rU; rA; rA; rG; rA; rU; rC; rC; | 109 |
| rU; rU | |
| rC; rA; rT; rT; rG; rT; rC; rT; rC; rC; rG; rA; rA; rG; rA; rA; rA; rT; rA; rA; rG; rA; rT; rC; rC; | 110 |
| rU; rU | |
| rC; rA; rU; rU; rG; rU; rC; rU; rC; rC; rG; rA; rA; rG; rA; rA; rA; rU; rA; rA; rG; rA; rU; omeC; | 111 |
| omeC; rU; rU | |
| rC; rA; rU; rU; rG; rU; rC; rU; rC; rC; rG; rA; rA; rG; rA; rA; rA; rU; rA; rA; rG; rA; rU; rC; rC; | 112 |
| rU; rU | |
| rC; rA; rU; rU; rG; rU; rC; rU; rC; rC; rG; rA; rA; rG; rA; rA; rA; rU; rA; rA; rG; rA; rU; rC; rC; | 113 |
| rU; rU | |
| rC; rA; rU; rU; rG; rU; rC; rU; rC; rC; rG; rA; rA; rG; rA; rA; rA; rU; rA; rA; rG; rA; rU; rC; rC; | 114 |
| rU; rU | |
| rC; rA; rT; rT; rG; rT; rC; rT; rC; rC; rG; rA; rA; rG; rA; rA; rA; rT; rA; rA; rG; rA; rT; rC; rC; | 115 |
| rT; rT | |
| rC; rA; rT; rT; rG; rT; rC; rT; rC; rC; rG; rA; rA; rG; rA; rA; rA; rT; rA; rA; rG; rA; rT; rC; rC; | 116 |
| rU; rU | |

A multi-letter code shown in Table 3 has been used in describing the siRNA structures in Tables 1 and 2 to unambiguously represent each sequence. In this code, the prefix “ribo” or “r” signifies an RNA nucleoside. The prefixes “d,” “ome,” and “r” refer only to the letter immediately following the prefix.

TABLE 3
siRNA Nucleotide Structure Code

| Code | Represents |
|---|---|
| Cy5 | Cy5 fluorescent dye |
| dA | deoxyadenosine |
| dC | deoxycytidine |
| dG | deoxyguanosine |
| dT | deoxythymidine |
| omeA | 2′-O-methyl adenosine |
| omeC | 2′-O-methyl cytidine |
| omeG | 2′-O-methyl guanosine |
| omeU | 2′-O-methyl uridine |
| p | 5′ phosphate |
| rA | (ribo)adenosine |
| rC | (ribo)cytidine |
| rG | (ribo)guanosine |
| rI | (ribo)inosine |
| rT | (ribo)thymidine (5-methyluridine) |
| rU | (ribo)uridine |

In Table 4, the top nine siRNA sites identified from laboratory screening studies have been identified.

TABLE 4

Top Nine siRNA Sites as Identified From
Laboratory Screening Studies; Showing Conserved
and Minor Variant 19-mer Sequences From the
Influenza A

| | | | | |
|---|---|---|---|---|
| SEQ ID | Ref | | | |
| NO: | ID | Segment | Match Sequence | % Total |

| | | | | |
|---|---|---|---|---|
| 117 | 7 | PB2 | AGACAGCGACCAAAAGAAU | 99.1% |
| 118 | 7 | | AGACAGCGACCAAAAGgAU | 0.3% |
| 119 | 7 | | AGACAGCGACCAAAgGAAU | 0.1% |
| 120 | 7 | | AGACAGCGACCAAAAGAuU | 0.1% |
| 121 | 7 | | AGACgaCGAuCAAAAGAAU | 0.1% |
| 122 | 17 | PB2 | ACUGACAGCCAGACAGCGA | 99.0% |
| 123 | 17 | | ACUGACAGuCAGACAGCGA | 0.2% |
| 124 | 17 | | ACUGAuAGCCAGACAGCGA | 0.3% |
| 125 | 17 | | ACcGACAGCCAGACAGCGA | 0.2% |
| 126 | 17 | | ACUGACAGCCAGACgaCGA | 0.1% |
| 127 | 48 | PB2 | CGGGACUCUAGCAUACUUA | 98.0% |
| 128 | 48 | | CGGGACUCUAGCAUgCUUA | 0.1% |
| 129 | 48 | | CGGGACUuUAGCAUACUUA | 0.2% |
| 130 | 48 | | aGGGACUCUAGCAUACUUA | 0.1% |
| 131 | 48 | | CGaGACUCUAGCAUACUUA | 0.4% |
| 132 | 48 | | CGGaACUCUAGCAUACUUA | 0.6% |
| 133 | 48 | | CGGGACUaUAGCAUACUUA | 0.1% |
| 134 | 48 | | CGGGACUCcAGCAUACUUA | 0.3% |
| 135 | 48 | | CGGGACUCUAaCAUACUUA | 0.1% |
| 136 | 1187 | PB1 | GAUCUGUUCCACCAUUGAA | 90.9% |
| 137 | 1187 | | GAUCUGUUuCACCAUUGAA | 0.7% |
| 138 | 1187 | | aAUCUGUUCCACCAUUGAA | 0.1% |
| 139 | 1187 | | GAcCUGUUCCACCAUUGAA | 6.9% |
| 140 | 1187 | | GAUCUGcUCCACCAUUGAA | 0.2% |
| 141 | 1187 | | GAUCUGUUaCACCAUUGAA | 0.1% |
| 142 | 1187 | | GAUCUGUUCCACCAUUaAA | 0.1% |
| 143 | 1187 | | GAcCUGUUCuACCAUUGAA | 0.1% |
| 144 | 1187 | | GAcCUGcUCCACCAUUGAA | 0.3% |
| 145 | 1206 | PB1 | AUGAAGAUCUGUUCCACCA | 88.6% |
| 146 | 1206 | | AUGAAGAUCUGUUuCACCA | 0.7% |
| 147 | 1206 | | AUGAgGAUCUGUUCCACCA | 0.2% |
| 148 | 1206 | | AcGAAGAUCUGUUCCACCA | 0.1% |
| 149 | 1206 | | AUGAAaAUCUGUUCCACCA | 0.1% |
| 150 | 1206 | | AUGAAGAcCUGUUCCACCA | 6.8% |
| 151 | 1206 | | AUGAAGAUCUGcUCCACCA | 0.2% |
| 152 | 1206 | | AUGAAGAUCUGUUaCACCA | 0.1% |
| 153 | 1206 | | cUGAAGAUCUGUUCCACCA | 0.1% |
| 154 | 1206 | | uUGAAGAUCUGUUCCACCA | 2.0% |
| 155 | 1206 | | AUGAAGAcCUGUUCuACCA | 0.1% |
| 156 | 1206 | | AUaAAGAcCUGUUCCACCA | 0.1% |
| 157 | 1206 | | AUGAAGAcCUGcUCCACCA | 0.2% |
| 158 | 2393 | PA | UUGAGGAGUGCCUGAUUAA | 98.7% |
| 159 | 2393 | | UUGAGGAGUGCCUGgUUAA | 0.1% |
| 160 | 2393 | | UUGAGGAaUGCCUGAUUAA | 1.0% |
| 161 | 2393 | | UUGAGGAGUGCCUaAUUAA | 0.2% |
| 162 | 2394 | PA | GCAAUUGAGGAGUGCCUGA | 98.6% |
| 163 | 2394 | | GCAAUUGAGGAGUGCCUGg | 0.1% |
| 164 | 2394 | | GCAgUUGAGGAGUGCCUGA | 0.1% |
| 165 | 2394 | | GCAAUUGAGGAaUGCCUGA | 1.0% |
| 166 | 2394 | | GCAAUUGAGGAGUGCCUaA | 0.2% |
| 167 | 3560 | NP | GAUCUUAUUUCUUCGGAGA | 96.0% |
| 168 | 3560 | | GAUCUUAUUUCUUCGGgGA | 1.7% |
| 169 | 3560 | | GAUCUUAUUUCUUuGGAGA | 0.2% |
| 170 | 3560 | | GgUCUUAUUUCUUCGGAGA | 0.9% |
| 171 | 3560 | | GuUCUUAUUUCUUCGGAGA | 1.1% |
| 172 | 3561 | NP | GGAUCUUAUUUCUUCGGAG | 96.0% |
| 173 | 3561 | | GGAUCUUAUUUCUUCGGgG | 1.7% |
| 174 | 3561 | | GGAUCUUAUUUCUUuGGAG | 0.2% |
| 175 | 3561 | | GGgUCUUAUUUCUUCGGAG | 0.9% |
| 176 | 3561 | | GGuUCUUAUUUCUUCGGAG | 1.1% |

Madin-Darby canine kidney cells (MDCK) were used to test siRNAs. For electroporation, the cells were kept in serum-free RPMI 1640 medium. Virus infections were done in infection medium. Influenza viruses A/PR/8/34 (PR8) and A/WSN/33 (WSN), subtypes H1N1 were used. Sense and antisense sequences that were tested are listed in Table 5.

TABLE 5

siRNA Sequences

| | |
|---|---|
| Name | siRNA Sequence (5′-3′) |

| | | |
|---|---|---|
| PB2-2210/2230 (sense) | ggagacgugguguugguaadTdT | (SEQ ID NO: 177) |
| PB2-2210/2230 (antisense) | uuaccaacaccacgucuccdTdT | (SEQ ID NO: 178) |
| PB2-2240/2260 (sense) | cgggacucuagcauacuuadTdT | (SEQ ID NO: 179) |
| PB2-2240/2260 (antisense) | uaaguaugcuagagucccgdTdT | (SEQ ID NO: 180) |
| PB1-6/26 (sense) | gcaggcaaaccauuugaaudTdT | (SEQ ID NO: 181) |
| PB1-6/26 (antisense) | auucaaaugguuugccugcdTdT | (SEQ ID NO: 182) |
| PB1-129/149 (sense) | caggauacaccauggauacdTdT | (SEQ ID NO: 183) |
| PB1-129/149 (antisense) | guauccaugguguauccugdTdT | (SEQ ID NO: 184) |
| PB1-2257/2277 (sense) | gaucuguuccaccauugaadTdT | (SEQ ID NO: 185) |
| PB1-2257/2277 (antisense) | uucaaugguggaacagaucdTdT | (SEQ ID NO: 186) |
| PA-44/64 (sense) | ugcuucaauccgaugauugdTdT | (SEQ ID NO: 187) |
| PA-44/64 (antisense) | caaucaucggauugaagcadTdT | (SEQ ID NO: 188) |
| PA-739/759 (sense) | cggcuacauugagggcaagdTdT | (SEQ ID NO: 189) |
| PA-739/759 (antisense) | cuugcccucaauguagccgdTdT | (SEQ ID NO: 190) |
| PA-2087/2107 (G) (sense) | gcaauugaggagugccugadTdT | (SEQ ID NO: 191) |
| PA-2087/2107 (G) (antisense) | ucaggcacuccucaauugcdTdT | (SEQ ID NO: 192) |
| PA-2110/2130 (sense) | ugaucccuggguuuugcuudTdT | (SEQ ID NO: 193) |
| PA-2110/2130 (antisense) | aagcaaaacccagggaucadTdT | (SEQ ID NO: 194) |
| PA-2131/2151 (sense) | ugcuucuugguucaacuccdTdT | (SEQ ID NO: 195) |
| PA-2131/2151 (antisense) | ggaguugaaccaagaagcadTdT | (SEQ ID NO: 196) |
| NP-231/251 (sense) | uagagagaauggugcucucdTdT | (SEQ ID NO: 197) |
| NP-231/251 (antisense) | gagagcaccauucucucuadTdT | (SEQ ID NO: 198) |
| NP-390/410 (sense) | uaaggcgaaucuggcgccadTdT | (SEQ ID NO: 199) |
| NP-390/410 (antisense) | uggcgccagauucgccuuadTdT | (SEQ ID NO: 200) |
| NP-1496/1516 (sense) | ggaucuuauuucuucggagdTdT | (SEQ ID NO: 201) |
| NP-1496/1516 (antisense) | cuccgaagaaauaagauccdTdT | (SEQ ID NO: 202) |
| NP-1496/1516a (sense) | ggaucuuauuucuucggagadTdT | (SEQ ID NO: 203) |
| NP-1496/1516a (antisense) | ucuccgaagaaauaagauccdTdT | (SEQ ID NO: 204) |
| M-37/57 (sense) | ccgaggucgaaacguacgudTdT | (SEQ ID NO: 205) |
| M-37/57 (antisense) | acguacguuucgaccucggdTdT | (SEQ ID NO: 206) |
| M-480/500 (sense) | cagauugcugacucccagcdTdT | (SEQ ID NO: 207) |
| M-480/500 (antisense) | gcugggagucagcaaucugdTdT | (SEQ ID NO: 208) |
| M-598/618 (sense) | uggcuggaucgagugagcadTdT | (SEQ ID NO: 209) |
| M-598/618 (antisense) | ugcucacucgauccagccadTdT | (SEQ ID NO: 210) |
| M-934/954 (sense) | gaauaucgaaaggaacagcdTdT | (SEQ ID NO: 211) |
| M-934/954 (antisense) | gcuguuccuuucgauauucdTdT | (SEQ ID NO: 212) |
| NS-128/148 (sense) | cggcuucgccgagaucagadAdT | (SEQ ID NO: 213) |
| NS-128/148 (antisense) | ucugaucucggcgaagccgdAdT | (SEQ ID NO: 214) |
| NS-562/582 (R) (sense) | guccuccgaugaggacuccdTdT | (SEQ ID NO: 215) |
| NS-562/582 (R) (antisense) | ggaguccucaucggaggacdTdT | (SEQ ID NO: 216) |
| NS-589/609 (sense) | ugauaacacaguucgagucdTdT | (SEQ ID NO: 217) |
| NS-589/609 (antisense) | gacucgaacuguguuaucadTdT | (SEQ ID NO: 218) |

All siRNAs were synthesized by Dharmacon Research (Lafayette, Colo.) using 2′ACE protection chemistry and transfected into the cells by electroporation. Six to eight hours following electroporation, the serum-containing medium was washed away and PR8 or WSN virus at the appropriate multiplicity of infection was inoculated into the wells. Cells were infected with either 1,000 PFU (one virus per 1,000 cells; MOI=0.001) or 10,000 PFU (one virus per 100 cells; MOI=0.01) of virus. After 1 hour incubation at room temperature, 2 ml of infection medium with 4 μg/ml of trypsin was added to each well and the cells were incubated and, at indicated times, supernatants were harvested from infected cultures and the titer of virus was determined by hemagglutination of chicken erythrocytes.

Supernatants were harvested at 24, 36, 48, and 60 hours after infection. Viral titer was measured using a standard hemagglutinin assay as described in Knipe, D. M. and P. M. Howley, Fundamental Virology, 4th ed., pp. 34-35. The hemagglutination assay was done in V-bottomed 96-well plates. Serial 2-fold dilutions of each sample were incubated for 1 h on ice with an equal volume of a 0.5% suspension of chicken erythrocytes (Charles River Laboratories). Wells containing an adherent, homogeneous layer of erythrocytes were scored as positive. For plaque assays, serial 10-fold dilutions of each sample were titered for virus as described in Fundamental Virology, 4th ed., p. 32, as well known in the art.

To investigate the feasibility of using siRNA to suppress influenza virus replication, various influenza virus A RNAs were targeted. Specifically, the MDCK cell line, which is permissive to influenza infection is widely used to study influenza virus, was utilized.

Each siRNA was individually introduced into populations of MDCK cells by electroporation. The following siRNA targeted to GFP was used as control:

SEQ ID NO: 219

| | |
|---|---|
| sense: | 5′-GGCUACGUCCAGGAGCGCAUU-3′ |

SEQ ID NO: 220

| | |
|---|---|
| antisense: | 5′-UGCGCUCCUGGACGUAGCCUU-3′ |

This siRNA is referred to as GFP-949. In subsequent experiments the UU overhang at the 3′ end of both strands was replaced by dTdT with no effect on results. A mock electroporation was also performed as a control. Eight hours after electroporation cells were infected with either influenza A virus PR8 or WSN at an MOI of either 0.1 or 0.01 and were analyzed for virus production at various time points (24, 36, 48, 60 hours) thereafter using a standard hemagglutination assay. GFP expression was assayed by flow cytometry using standard methods.

The ability of individual dsRNAs to inhibit replication of influenza virus A strain A/Puerto Rico/8/34 (H1N1) or influenza virus A strain A/WSN/33 (H1N1) was determined by measuring HA titer. Thus a high HA titer indicates a lack of inhibition while a low HA titer indicates effective inhibition. MDCK cells were infected at an MOI of 0.01. For these experiments one siRNA that targets the PB1 segment (PB1-2257/2277), one siRNA that targets the PB2 segment (PB2-2240/2260), one siRNA that targets the PA segment (PA-2087/2107 (G)), and three different siRNAs that target the NP genome and transcript (NP-231/251, NP-390/410, and NP-1496/1516) were tested.

In the absence of siRNA (mock TF) or the presence of control (GFP) siRNA, the titer of virus increased over time, reaching a peak at approximately 48-60 hours after infection. In contrast, at 60 hours the viral titer was significantly lower in the presence of any of the siRNAs. For example, in strain WSN the HA titer (which reflects the level of virus) was approximately half as great in the presence of siRNAs PB2-2240 or NP-231 than in the controls. In particular, the level of virus was below the detection limit (10,000 PFU/ml) in the presence of siRNA NP-1496 in both strains. This represents a decrease by a factor of more than 60-fold in the PR8 strain and more than 120-fold in the WSN strain. The level of virus was also below the detection limit (10,000 PFU/ml) in the presence of siRNA PA-2087(G) in strain WSN and was extremely low in strain PR8. Suppression of virus production by siRNA was evident even from the earliest time point measured. Effective suppression, including suppression of virus production to undetectable levels (as determined by HA titer) has been observed at time points as great as 72 hours post-infection.

A total of twenty siRNAs, targeted to 6 segments of the influenza virus genome (PB2, PB1, PA, NP, M and NS), were tested in the MDCK cell line system. About 15% of the siRNA (PB1-2257, PA-2087G and NP-1496) tested displayed a strong effect, inhibiting viral production by more than 100-fold in most cases at MOI=0.001 and by 16 to 64 fold at MOI=0.01, regardless of whether PR8 or WSN virus was used. In particular, when siRNA NP-1496 or PA-2087 was used, inhibition was so pronounced that culture supernatants lacked detectable hemagglutinin activity. These potent siRNAs target 3 different viral gene segments: PB1 and PA, which are involved in the RNA transcriptase complex, and NP which is a single-stranded RNA binding nucleoprotein. Consistent with findings in other systems, the sequences targeted by these siRNAs are all positioned relatively close to the 3-prime end of the coding region.

Approximately 40% of the siRNAs significantly inhibited virus production, but the extent of inhibition varied depending on certain parameters. Approximately 15% of siRNAs potently inhibited virus production regardless of whether PR8 or WSN virus was used. However, in the case of certain siRNAs, the extent of inhibition varied somewhat depending on whether PR8 or WSN was used. Some siRNAs significantly inhibited virus production only at early time points (24 to 36 hours after infection) or only at lower dosage of infection (MOI=0.001), such as PB2-2240, PB1-129, NP-231 and M-37. These siRNAs target different viral gene segments, and the corresponding sequences are positioned either close to 3-prime end or 5-prime end of the coding region.

Approximately 45% of the siRNAs had no discernible effect on the virus titer, indicating that they were not effective in interfering with influenza virus production in MDCK cells. In particular, none of the four siRNAs which target the NS gene segment showed any inhibitory effect.

To estimate virus titers more precisely, plaque assays with culture supernatants were performed (at 60 hours) from culture supernatants obtained from virus-infected cells that had undergone mock transfection or transfection with NP-1496. Approximately 6×10⁵ pfu/ml was detected in mock supernatant, whereas no plaques were detected in undiluted NP-1496 supernatant. As the detection limit of the plaque assay is about 20 pfu (plaque forming unit)/ml, the inhibition of virus production by NP-1496 is at least about 30,000 fold. Even at an MOI of 0.1, NP-1496 inhibited virus production about 200-fold.

To determine the potency of siRNA, a graded amount of NP-1496 was transfected into MDCK cells followed by infection with PR8 virus. Virus titers in the culture supernatants were measured by hemagglutinin assay. As the amount of siRNA decreased, virus titer increased in the culture supernatants. However, even when as little as 25 pmol of siRNA was used for transfection, approximately 4-fold inhibition of virus production was detected as compared to mock transfection, indicating the potency of NP-1496 siRNA in inhibiting influenza virus production.

In a typical influenza virus infection, new virions are released beginning at about 4 hours after infection. To determine whether siRNA could reduce or eliminate infection by newly released virus in the face of an existing infection, MDCK cells were infected with PR8 virus and then transfected with NP-1496 siRNA. Virus titer increased steadily over time following mock transfection, whereas virus titer increased only slightly in NP-1496 transfected cells. Thus administration of siRNA after virus infection is effective.

Together, these results show that (i) certain siRNAs can potently inhibit influenza virus production; (ii) influenza virus production can be inhibited by siRNAs specific for different viral genes, including those encoding NP, PA, and PB1 proteins; and (iii) siRNA inhibition occurs in cells that were infected previously in addition to cells infected simultaneously with or following administration of siRNAs.

### EXAMPLE 3

### siRNAs that Target Viral RNA Polymerase or Nucleoprotein Inhibit Influenza A Virus Production in Chicken Embryos

For siRNA-oligofectamine complex formation and chicken embryo inoculation, siRNAs were prepared as described above. Chicken eggs were maintained under standard conditions. 30 μl of Oligofectamine (product number: 12252011 from Life Technologies, now Invitrogen) was mixed with 30 μl of Opti-MEM I (Gibco) and incubated at RT for 5 min. 2.5 nmol (10 μl) of siRNA was mixed with 30 μl of Opti-MEM I and added into diluted oligofectamine. The siRNA and oligofectamine was incubated at RT for 30 minutes. 10-day old chicken eggs were inoculated with siRNA-oligofectamine complex together with 100 μl of PR8 virus (5000 pfu/ml). The eggs were incubated at 37° C. for indicated time and allantoic fluid was harvested. Viral titer in allantoic fluid was tested by HA assay as described above.

To confirm the results in MDCK cells, the ability of siRNA to inhibit influenza virus production in fertilized chicken eggs was also assayed. Because electroporation cannot be used on eggs, Oligofectamine, a lipid-based agent that has been shown to facilitate intracellular uptake of DNA oligonucleotides as well as siRNAs in vitro was used (25). Briefly, PR8 virus alone (500 pfu) or virus plus siRNA-oligofectamine complex was injected into the allantoic cavity of 10-day old chicken eggs. Allantoic fluids were collected 17 hours later for measuring virus titers by hemagglutinin assay. When virus was injected alone (in the presence of Oligofectamine), high virus titers were readily detected. Co-injection of GFP-949 did not significantly affect the virus titer. (No significant reduction in virus titer was observed when Oligofectamine was omitted.)

The injection of siRNAs specific for influenza virus showed results consistent with those observed in MDCK cells: The same siRNAs (NP-1496, PA2087 and PB1-2257) that inhibited influenza virus production in MDCK cells also inhibited virus production in chicken eggs, whereas the siRNAs (NP-231, M-37 and PB1-129) that were less effective in MDCK cells were ineffective in fertilized chicken eggs. Thus, siRNAs are also effective in interfering with influenza virus production in fertilized chicken eggs.

### EXAMPLE 4

### siRNA Inhibits Influenza Virus Production at the mRNA Level

siRNA preparation was performed as described above. For RNA extraction, reverse transcription and real time PCR, 1×10⁷ MDCK cells were electroporated with 2.5 nmol of NP-1496 or mock electroporated (no siRNA). Eight hours later, influenza A PR8 virus was inoculated into the cells at MOI=0.1. At times 1, 2, and 3-hour post-infection, the supernatant was removed, and the cells were lysed with Trizol reagent (Gibco). RNA was purified according to the manufacturer's instructions. Reverse transcription (RT) was carried out at 37° C. for 1 hour, using 200 ng of total RNA, specific primers (see below), and Omniscript Reverse transcriptase kit (Qiagen) in a 20-μl reaction mixture according to the manufacturer's instructions. Primers specific for either mRNA, NP vRNA, NP cRNA, NS vRNA, or NS cRNA were as follows:

mRNA; dT₁₈:

SEQ ID NO: 221

5′-TTTTTTTTTTTTTTTTTT-3′
NP vRNA, NP-367:

SEQ ID NO: 222

5′-CTCGTCGCTTATGACAAAGAAG-3′
NP cRNA, NP-1565R:

SEQ ID NO: 223

5′-ATATCGTCTCGTATTAGTAGAAACAAGGGTATTTTT-3′
NS vRNA, NS-527:

SEQ ID NO: 224

5′-CAGGACATACTGATGAGGATG-3′
NS cRNA, NS-890R:

SEQ ID NO: 225

5′-ATATCGTCTCGTATTAGTAGAAACAAGGGTGTTTT-3′

1 μl of RT reaction mixture (i.e, the sample obtained by performing reverse transcription) and sequence-specific primers were used for real-time PCR using SYBR Green PCR master mix (AB Applied Biosystems) including SYBR Green I double-stranded DNA binding dye. PCRs were cycled in an ABI PRISM 7000 sequence detection system (AB applied Biosystem) and analyzed with ABI PRISM 7000 SDS software (AB Applied Biosystems). The PCR reaction was carried out at 50° C., 2 minutes, 95° C., 10 minutes, then 95° C., 15 seconds and 60° C., 1 minute for 50 cycles. Cycle times were analyzed at a reading of 0.2 fluorescence units. All reactions were done in duplicate. Cycle times that varied by more than 1.0 between the duplicates were discarded. The duplicate cycle times were then averaged and the cycle time of β-actin was subtracted from them for a normalized value.

PCR primers were as follows:

| | |
|---|---|
| For NP RNAs | |
| NP-367: | |
| 5′-CTCGTCGCTTATGACAAAGAAG-3′. | (SEQ ID NO: 226) |
| NP-460R: | |
| 5′-AGATCATCATGTGAGTCAGAC-3′. | (SEQ ID NO: 227) |
| For NS RNAs: | |
| NS-527: | |
| 5′-CAGGACATACTGATGAGGATG-3′. | (SEQ ID NO: 228) |
| NS-617R: | |
| 5′-GTTTCAGAGACTCGAACTGTG-3′. | (SEQ ID NO: 229) |

As described above, during replication of influenza virus, vRNA is transcribed to produce cRNA, which serves as a template for more vRNA synthesis, and mRNA, which serves as a template for protein synthesis (1). Although RNAi is known to target the degradation of mRNA in a sequence-specific manner (16-18), there is a possibility that vRNA and cRNA are also targets for siRNA since vRNA of influenza A virus is sensitive to nuclease (1). To investigate the effect of siRNA on the degradation of various RNA species, reverse transcription using sequence-specific primers followed by real time PCR was used to quantify the levels of vRNA, cRNA and mRNA. The cRNA is the exact complement of vRNA, but mRNA contains a polyA sequence at the 3′ end, beginning at a site complementary to a site 15-22 nucleotides downstream from the 5′ end of the vRNA segment. Thus compared to vRNA and cRNA, mRNA lacks 15 to 22 nucleotides at the 3′ end. To distinguish among the three viral RNA species, primers specific for vRNA, cRNA and mRNA were used in the first reverse transcription reaction. For mRNA, poly dT18 was used as primer. For cRNA, a primer complementary to the 3′ end of the RNA that is missing from mRNA was used. For vRNA, the primer can be almost anywhere along the RNA as long as it is complementary to vRNA and not too close to the 5′ end. The resulting cDNA transcribed from only one of the RNAs was amplified by real time PCR.

Following influenza virus infection, new virions are starting to be packaged and released by about 4 hours. To determine the effect of siRNA on the first wave of mRNA and cRNA transcription, RNA was isolated early after infection. Briefly, NP-1496 was electroporated into MDCK cells. A mock electroporation (no siRNA) was also performed). Six to eight hours later, cells were infected with PR8 virus at MOI=0.1. The cells were then lysed at 1, 2 and 3 hours post-infection and RNA was isolated. The levels of mRNA, vRNA and cRNA were assayed by reverse transcription using primers for each RNA species, followed by real time PCR.

One hour after infection, there was no significant difference in the amount of NP mRNA between samples with or without NP siRNA transfection. As early as 2 hours post-infection, NP mRNA increased by 38 fold in the mock transfection group, whereas the levels of NP mRNA did not increase (or even slightly decreased) in cells transfected with siRNA. Three hours post-infection, mRNA transcript levels continued to increase in the mock transfection whereas a continuous decrease in the amount of NP mRNA was observed in the cells that received siRNA treatment. NP vRNA and cRNA displayed a similar pattern except that the increase in the amount of vRNA and cRNA in the mock transfection was significant only at 3 hours post-infection.

These results indicate that, consistent with the results of measuring intact, live virus by hemagglutinin assay or plaque assay, the amounts of all NP RNA species were also significantly reduced by the treatment with NP siRNA.

### EXAMPLE 5

### Inhibition of Influenza Virus Production in Mice by siRNAs

This example describes experiments showing that administration of siRNAs targeted to influenza virus NP or PA transcripts inhibit production of influenza virus in mice when administered either prior to or following infection with influenza virus. The inhibition is dose-dependent and shows additive effects when two siRNAs each targeted to a transcript expressed from a different influenza virus gene were administered together.

### Materials and Methods:

siRNA preparation was performed as described above. For siRNA delivery, siRNAs (30 or 60 μg of GFP-949, NP-1496, or PA-2087) were incubated with jetPEI™ for oligonucleotides cationic polymer transfection reagent, N/P ratio=5 (Qbiogene, Inc., Carlsbad, Calif.; Cat. No. GDSP20130; N/P refers to the number of nitrogens per nucleotide phosphate in the jetPEI/siRNA mixture) or with poly-L-lysine (MW (vis) 52,000; MW (LALLS) 41,800, Sigma Cat. No. P2636) for 20 minutes at room temperature in 5% glucose. The mixture was injected into mice intravenously, into the retro-orbital vein, 200 μl per mouse, 4 mice per group. 200 μl 5% glucose was injected into control (no treatment) mice. The mice were anesthetized with 2.5% Avertin before siRNA injection or intranasal infection.

For viral infection, B6 mice were intranasally infected with PR8 virus by dropping virus-containing buffer into the mouse's nose with a pipette, 301 (12,000 pfu) per mouse.

For determination of viral titer, mice were sacrificed at various times following infection, and lungs were harvested. Lungs were homogenized, and the homogenate was frozen and thawed twice to release virus. PR8 virus present in infected lungs was titered by infection of MDCK cells. Flat-bottom 96-well plates were seeded with 3×10⁴ MDCK cells per well, and 24 hours later the serum-containing medium was removed. 25 μl of lung homogenate, either undiluted or diluted from 1×10⁻¹ to 1×10⁻⁷, was inoculated into triplicate wells. After 1 hour incubation, 175 μl of infection medium with 4 μg/ml of trypsin was added to each well. Following a 48 h incubation at 37° C., the presence or absence of virus was determined by hemagglutination of chicken RBC by supernatant from infected cells. The hemagglutination assay was carried out in V-bottom 96-well plates. Serial 2-fold dilutions of supernatant were mixed with an equal volume of a 0.5% suspension (vol/vol) of chicken erythrocytes (Charles River Laboratories) and incubated on ice for 1 hour. Wells containing an adherent, homogeneous layer of erythrocytes were scored as positive. The virus titers were determined by interpolation of the dilution end point that infected 50% of wells by the method of Reed and Muench (TCID₅₀), thus a lower TCID₅₀ reflects a lower virus titer. The data from any two groups were compared by Student t test, which was used throughout the experiments described herein to evaluate significance.

siRNA targeted to viral NP transcripts inhibits influenza virus production in mice when administered prior to infection. 30 or 60 μg of GFP-949 or NP-1496 siRNAs were incubated with jetPEI and injected intravenously into mice as described above in Materials and Methods. Three hours later mice were intranasally infected with PR8 virus, 12000 pfu per mouse. Lungs were harvested 24 hours after infection. The average log₁₀TCID₅₀ of the lung homogenate for mice that received no siRNA treatment or received an siRNA targeted to GFP was 4.2. In mice that were pretreated with 30 μg siRNA targeted to NP and jetPEI, the average log₁₀TCID₅₀ of the lung homogenate was 3.9. In mice that were pretreated with 60 μg siRNA targeted to NP and jetPEI, the average log₁₀TCID₅₀ of the lung homogenate was 3.2. The difference in virus titer in the lung homogenate between the group that received no treatment and the group that received 60 μg NP siRNA was significant with P=0.0002. Data for individual mice are presented in Table 6.

siRNA targeted to viral NP transcripts inhibits influenza virus production in mice when administered intravenously prior to infection in a composition containing the cationic polymer PLL. 30 or 60 μg of GFP-949 or NP-1496 siRNAs were incubated with PLL and injected intravenously into mice as described above in Materials and Methods. Three hours later mice were intranasally infected with PR8 virus, 12000 pfu per mouse. Lungs were harvested 24 hours after infection. The average log₁₀TCID₅₀ of the lung homogenate for mice that received no siRNA treatment (NT) or received an siRNA targeted to GFP (GFP 60 μg) was 4.1. In mice that were pretreated with 60 μg siRNA targeted to NP (NP 60 μg) and PLL, the average log₁₀TCID₅₀ of the lung homogenate was 3.0. The difference in virus titer in the lung homogenate between the group that received 60 μg GFP and the group that received 60 μg NP siRNA was significant with P=0.001. Data for individual mice are presented in Table 6. These data indicate that siRNA targeted to the influenza NP transcript reduced the virus titer in the lung when administered prior to virus infection. They also indicate that a mixtures of an siRNA with a cationic polymer effectively inhibits influenza virus in the lung when administered by intravenous injection, not requiring techniques such as hydrodynamic transfection.

TABLE 6
Inhibition of Influenza Virus Production
in Mice by siRNA With Cationic Polymers

| | |
|---|---|
| Treatment | log₁₀TCID50 |

| | | | | |
|---|---|---|---|---|
| NT (jetPEI experiment) | 4.3 | 4.3 | 4.0 | 4.0 |
| GFP (60 μg) + jetPEI | 4.3 | 4.3 | 4.3 | 4.0 |
| NP (30 μg) + jetPEI | 4.0 | 4.0 | 3.7 | 3.7 |
| NP (60 μg) + jetPEI | 3.3 | 3.3 | 3.0 | 3.0 |
| NT (PLL experiment) | 4.0 | 4.3 | 4.0 | 4.0 |
| GFP (60 μg) + PLL | 4.3 | 4.0 | 4.0 | (not done) |
| NP (60 μg) + PLL | 3.3 | 3.0 | 3.0 | 2.7 |

siRNA targeted to viral NP transcripts inhibits influenza virus production in mice when administered prior to infection and demonstrates that the presence of a cationic polymer significantly increases the inhibitory efficacy of siRNA. 60 μg of GFP-949 or NP-1496 siRNAs were incubated with phosphate buffered saline (PBS) or jetPEI and injected intravenously into mice as described above in Materials and Methods. Three hours later mice were intranasally infected with PR8 virus, 12000 pfu per mouse. Lungs were harvested 24 hours after infection. The average log₁₀TCID₅₀ of the lung homogenate for mice that received no siRNA treatment was 4.1, while the average log₁₀TCID₅₀ of the lung homogenate for mice that received an siRNA targeted to GFP in PBS was 4.4. In mice that were pretreated with 60 μg siRNA targeted to NP in PBS the average log₁₀TCID₅₀ of the lung homogenate was 4.2, showing only a modest increase in efficacy relative to no treatment or treatment with an siRNA targeted to GFP. In mice that were pretreated with 60 μg siRNA targeted to GFP in jetPEI, the average log₁₀TCID₅₀ of the lung homogenate was 4.2. However, in mice that received 60 μg siRNA targeted to NP in jetPEI, the average log₁₀TCID₅₀ of the lung homogenate was 3.2. The difference in virus titer in the lung homogenate between the group that received GFP siRNA in PBS and the group that received NP siRNA in PBS was significant with P=0.04, while the difference in virus titer in the lung homogenate between the group that received GFP siRNA with jetPEI and the group that received NP siRNA with jetPEI was highly significant with P=0.003. Data for individual mice are presented in Table 7.

TABLE 7
Inhibition of Influenza Virus Production in
Mice by siRNA Showing Increased Efficacy With Cationic Polymer

| | |
|---|---|
| Treatment | log₁₀TCID50 |

| | | | | |
|---|---|---|---|---|
| NT | 4.3 | 4.3 | 4.0 | 3.7 |
| GFP (60 μg) + PBS | 4.3 | 4.3 | 4.7 | 4.3 |
| NP (60 μg) + PBS | 3.7 | 4.3 | 4.0 | 4.0 |
| GFP (60 μg) + jetPEI | 4.3 | 4.3 | 4.0 | 3.0 |
| NT (60 μg) + jetPEI | 3.3 | 3.0 | 3.7 | 3.0 |

Additional experiments were performed to assess the ability of siRNA to inhibit influenza virus production at various times after infection, when administered at various time points prior to or following infection.

siRNA was administered as described above except that 120 μg siRNA was administered 12 hours before virus infection. Table 8 shows the results expressed as log₁₀TCID₅₀. The P value comparing NP-treated with control group was 0.049.

TABLE 8

| | Mouse 1 | Mouse 2 | Mouse 3 | Mouse 4 |
|---|---|---|---|---|
| NT | 4.3 | 4 | 4 | 4 |
| GFP-949 | 4.3 | 4 | 4 | 4 |
| NP-1496 | 4 | 3.7 | 3.7 | 3.3 |

In another experiment, siRNA (60 μg) was administered 3 hours before infection. 1500 pfu of PR8 virus was administered intranasally. The infected lung was harvested 48 hours after infection. Table 9 shows the results expressed as log₁₀TCID₅₀. The P value comparing NP-treated with control group was 0.03.

TABLE 9

| | Mouse 1 | Mouse 2 | Mouse 3 | Mouse 4 |
|---|---|---|---|---|
| NT | 4 | 4 | 4 | 4 |
| GFP-949 | 4.3 | 4 | 4 | 3.7 |
| NP-1496 | 3 | 3.7 | 3.7 | 3.3 |

In another experiment, siRNA (120 μg) was administered 24 hours after PR8 (1500 pfu) infection. 52 hours post-infection, the lung was harvested and virus titer was measured. Table 10 shows the results expressed as log₁₀TCID₅₀. The P value comparing NP-treated with control group was 0.03.

TABLE 10

| | Mouse 1 | Mouse 2 | Mouse 3 | Mouse 4 |
|---|---|---|---|---|
| GFP-949 | 2.3 | 2.7 | 2 | 2.7 |
| NP-1496 | 2 | 2 | 1.7 | 2 |

siRNAs targeted to different influenza virus transcripts exhibit an additive effect. Sixty μg of NP-1496 siRNA, 60 μg PA-2087 siRNA, or 60 μg NP-1496 siRNA+60 μg PA-2087 siRNA were incubated with jetPEI and injected intravenously into mice as described above. Three hours later mice were intranasally infected with PR8 virus, 12000 pfu per mouse. Lungs were harvested 24 hours after infection. The average log₁₀TCID₅₀ of the lung homogenate for mice that received no siRNA treatment was 4.2. In mice that received 60 μg siRNA targeted to NP, the average log₁₀TCID₅₀ of the lung homogenate was 3.2. In mice that received 60 μg siRNA targeted to PA, the average log₁₀TCID₅₀ of the lung homogenate was 3.4. In mice that received 60 μg siRNA targeted to NP+60 μg siRNA targeted to PA, the average log₁₀TCID₅₀ of the lung homogenate was 2.4. The differences in virus titer in the lung homogenate between the group that received no treatment and the groups that received 60 μg NP siRNA, 60 μg PA siRNA, or 60 μg NP siRNA+60 μg PA siRNA were significant with P=0.003, 0.01, and 0.0001, respectively. The differences in lung homogenate between the groups that received 60 μg NP siRNA or 60 μg NP siRNA and the group that received 60 μg NP siRNA+60 μg PA siRNA were significant with p=0.01. Data for individual mice are presented in Table 11. These data indicate that pretreatment with siRNA targeted to the influenza NP or PA transcript reduced the virus titer in the lungs of mice subsequently infected with influenza virus. The data further indicate that a combination of siRNA targeted to different viral transcripts exhibit an additive effect, suggesting that therapy with a combination of siRNAs targeted to different transcripts may allow a reduction in dose of each siRNA, relative to the amount of a single siRNA that would be needed to achieve equal efficacy.

TABLE 11
Additive Effect of siRNA Against Influenza Virus in Mice

| | |
|---|---|
| Treatment | log₁₀TCID50 |

| | | | | |
|---|---|---|---|---|
| NT | 4.3 | 4.3 | 4.0 | 4.0 |
| NP (60 μg) | 3.7 | 3.3 | 3.0 | 3.0 |
| PA (60 μg) | 3.7 | 3.7 | 3.0 | 3.0 |
| NP + PA (60 μg each) | 2.7 | 2.7 | 2.3 | 2.0 |

siRNA targeted to viral NP transcripts inhibits influenza virus production in mice when administered following infection. Mice were intranasally infected with PR8 virus, 500 pfu. Sixty μg of GFP-949 siRNA, 60 μg PA-2087 siRNA, 60 μg NP-1496 siRNA, or 60 μg NP siRNA+60 μg PA siRNA were incubated with jetPEI and injected intravenously into mice 5 hours later as described above. Lungs were harvested 28 hours after administration of siRNA. The average log₁₀TCID₅₀ of the lung homogenate for mice that received no siRNA treatment or received the GFP-specific siRNA GFP-949 was 3.0. In mice that received 60 μg siRNA targeted to PA, the average log₁₀TCID₅₀ of the lung homogenate was 2.2. In mice that received 60 μg siRNA targeted to NP (NP 60 μg), the average log₁₀TCID₅₀ of the lung homogenate was 2.2. In mice that received 60 μg NP siRNA+60 μg PA siRNA, the average log₁₀TCID₅₀ of the lung homogenate was 1.8. The differences in virus titer in the lung homogenate between the group that received no treatment and the groups that received 60 μg PA, NP siRNA, or 60 μg NP siRNA+60 μg PA siRNA were significant with P=0.09, 0.02, and 0.003, respectively. The difference in virus titer in the lung homogenate between the group that received NP siRNA and PA+NP siRNAs had a P value of 0.2. Data for individual mice are presented in Table 12. These data indicate that siRNA targeted to the influenza NP and/or PA transcripts reduced the virus titer in the lung when administered following virus infection.

TABLE 12
Inhibition of Influenza Virus Production in Infected Mice by siRNA

| | |
|---|---|
| Treatment | log₁₀TCID50 |

| | | | | |
|---|---|---|---|---|
| NT | 3.0 | 3.0 | 3.0 | 3.0 |
| GFP (60 μg) | 3.0 | 3.0 | 3.0 | 2.7 |
| PA (60 μg) | 2.7 | 2.7 | 2.3 | 1.3 |
| NP (60 μg) | 2.7 | 2.3 | 2.3 | 1.7 |
| NP + PA (60 μg each) | 2.3 | 2.0 | 1.7 | 1.3 |

### EXAMPLE 6

### Inhibition of Luciferase Activity in the Lung by Delivery of siRNA to the Vascular System or the Respiratory Tract

siRNAs were obtained from Dharmacon and were deprotected and annealed as described above. siRNA sequences for NP (NP-1496), PA (PA-2087), PB1 (PB1-2257), and GFP were as given above. Luc-specific siRNA was as described in McCaffrey, A P, et al., Nature 418:38-39.

pCMV-luc DNA (Promega) was mixed with PEI (Qbiogene, Carlsbad, Calif.) at a nitrogen/phosphorus molar ratio (N/P ratio) of 10 at room temperature for 20 minutes. For I.V. administration, 200 μl of the mixture containing 60 μg of DNA was injected retroorbitally into 8 week old male C57BL/6 mice (Taconic Farms). For intratracheal (I.T.) administration, 50 μl of the mixture containing 30 μg or 60 μg of DNA was administered into the lungs of anesthetized mice using a Penn Century Model IA-IC insufflator.

siRNA-PEI compositions were formed by mixing 60 μg of luc-specific or GFP-specific siRNA with jetPEI at an N/P ratio of 5 at room temperature for 20 min. For I.V. administration, 200 μl of the mixture containing the indicated amounts of siRNA was injected retroorbitally. For pulmonary administration, 50 μl was delivered intratracheally.

At various times after pCMV-luc DNA administration, lungs, spleen, liver, heart, and kidney were harvested and homogenized in Cell Lysis Buffer (Marker Gene Technologies, Eugene, Oreg.). Luminescence was analyzed with the Luciferase Assay System (Promega) and measured with an Optocomp® I luminometer (MGM Instruments, Hamden, Conn.). The protein concentrations in homogenates were measured by the BCA assay (Pierce).

To determine the tissue distribution of PEI-mediated nucleic acid delivery in mice, pCMV-luc DNA-PEI complexes were injected i.v., and 24 hours later, Luc activity was measured in various organs. Activity was highest in the lungs, where Luc activity was detected for at least 4 days, whereas in heart, liver, spleen, and kidney, levels were 100-1,000 times lower and were detected for a shorter time after injection. When DNA-PEI complexes were instilled I.T., significant Luc activity was also detected in the lungs, although at a lower level than after I.V. administration.

To test the ability of PEI to promote uptake of siRNAs by the lungs following I.V. administration, mice were first given pCMV-luc DNA-PEI complexes I.T., followed by I.V. injection of Luc-specific siRNA complexed with PEI, control GFP-specific siRNA complexed with PEI, or the same volume of 5% glucose. Twenty-four hours later, Luc activity in the lungs was 17-fold lower in mice that received Luc siRNA than in those given GFP siRNA or no treatment. Because Luc siRNA can inhibit Luc expression only in the same lung cells that were transfected with the DNA vector, these results indicate that I.V. injection of a siRNA-PEI mixture achieves effective inhibition of a target transcript in the lung.

To test the ability of PEI to promote uptake of siRNAs by the lungs following pulmonary administration, mice were first given pCMVDNA-PEI complexes I.V., followed immediately by I.T. administration of Luc-specific siRNA mixed with PEI, control GFP-specific siRNA mixed with PEI, or the same volume of 5% glucose. Twenty-four hours later, luciferase activities were assayed in lung homogenates. Luciferase activity was 6.8-fold lower in mice that were treated with luciferase siRNA than those treated with GFP siRNA. These results indicate that pulmonary administration of an siRNA-PEI mixture achieves effective inhibition of a target transcript in lung cells.

### EXAMPLE 7

### Inhibition of Cyclophilin B in the Lung by Delivery of siRNA to the Respiratory System

Cyclophilin B is an endogenous gene that is widely expressed in mammals. To assess the ability of siRNA delivered directly to the respiratory system to inhibit expression of an endogenous gene, outbred Blackswiss mice (around 30 g or more body weight) were anesthetized by isofluorane/oxygen, and siRNA targeted to cyclophilin B (Dharmacon, D-001136-01-20 siCONTROL Cyclophilin B siRNA (Human/Mouse/Rat) or control GFP-949 siRNA (2 mg/kg) was administered intranasally to groups of 2 mice for each siRNA. Lungs were harvested 24 hours after administration. RNA was extracted from the lung and reverse transcription was done using a random primer. Real time PCR was then performed using cyclophilin B and GAPDH Taqman gene expression assay (Applied Biosystems). Results (Table 13) showed 70% silencing of cyclophilin B by siRNA targeted to cyclophilin B.

TABLE 13
Inhibition of Cyclophilin B in the Lung

| Average | Normalized | Ave normal | Silencing % |
|---|---|---|---|
| PBS-1 | 5.395406 | 4.288984 | |
| PBS-2 | 3.182562 | | |
| GFP-1 | 2.547352 | 3.752446 | 12.50968 |
| GFP-2 | 4.957539 | | |
| Cyclo-1 | 1.173444 | 1.256672 | 70.7 |
| Cyclo-2 | 1.339901 | | |

TABLE 14

Target Portions in NP Gene

| | | |
|---|---|---|
| | | Nucleo- |
| ID | | tide |
| Number | Sequence | Position |

| | | | |
|---|---|---|---|
| 1 | agcaaaagcaggguagaua | (SEQ ID NO: 230) | 1-19 |
| 2 | gcaaaagcaggguagauaa | (SEQ ID NO: 231) | 2-20 |
| 3 | caaaagcaggguagauaau | (SEQ ID NO: 232) | 3-21 |
| 4 | aaaagcaggguagauaauc | (SEQ ID NO: 233) | 4-22 |
| 5 | aaagcaggguagauaauca | (SEQ ID NO: 234) | 5-23 |
| 6 | aagcaggguagauaaucac | (SEQ ID NO: 235) | 6-24 |

### EXAMPLE 8

### Inhibition of Influenza Virus by Direct Delivery of Naked siRNA to the Respiratory System

siRNA preparation, viral infection, lung harvests, and influenza virus titer assays were performed as described above. Mice were anesthetized using isofluorane (administered by inhalation). siRNA was delivered in a volume of 50 μl by intranasal drip. P values were computed using Student's T test.

siRNA (NP-1496) in phosphate buffered saline (PBS) was administered to groups of mice (5 mice per group). Mice were infected with influenza virus (2000 PFU) 3 hours after siRNA administration. Lungs were harvested 24 hours post-infection and virus titer measured. In a preliminary experiment mice were anesthetized with avertin and 2 mg/kg siRNA was administered by intranasal drip. A reduction in virus titer relative to controls was observed, although it did not reach statistical significance (data not shown). In a second experiment, Black Swiss mice were anesthetized using isofluorane/O₂. Various amounts of siRNA in PBS was intranasally administered into the mice., 50 μl each mouse. Three different groups (5 mice per group) received doses of 2 mg/kg, 4 mg/kg, or 10 mg/kg siRNA in PBS by intranasal drip. A fourth group that received PBS alone served as a control. Three hours later, the mice were anesthetized again using isofluorane/O₂, 30 μl of PR8 virus (2000 pfu=4× lethal dose) was intranasally administered into the mice. 24 hours after infection, the mouse lungs were harvested, homogenized and virus titer was measured by evaluation of the TCID₅₀ as described above. Serial 5-fold dilutions of the lung homogenate were performed rather than 10-fold dilutions.

A significant and dose-dependent difference in virus titer was seen between mice in each of the three treated groups and the controls (Table 15). The reduction in virus titer relative to controls was 3.45-fold (p=0.0125), 4.16-fold (p=0.0063), and 4.62-fold (p=0.0057) in the groups that received doses of 2 mg/kg, 4 mg/kg, and 10 mg/kg respectively. In summary, these results demonstrate the efficacy of siRNA delivered to the respiratory system in an aqueous medium in the absence of specific agents to enhance delivery.

TABLE 15
Intranasal Delivery of Naked siRNA Inhibits Influenza Virus Production

| | | | |
|---|---|---|---|
| Treatment | log₁₀TCID50 | Average | P value |

| | | | | | | | |
|---|---|---|---|---|---|---|---|
| PBS | 26718.37 | 45687.78 | 45687.78 | 15625 | 26718.37 | 32087.46 | |
| NP (2 mg/kg) | 15625 | 15625 | 3125 | 3125 | 9137.56 | 9327.51 | 0.008 |
| NP (4 mg/kg) | 9137.56 | 9137.56 | 5343.68 | 9137.56 | 5343.68 | 7620 | 0.004 |
| NP (10 mg/kg) | 9137.56 | 9137.56 | 9137.56 | 3125 | 3125 | 6732.53 | 0.003 |

### EXAMPLE 9

### Inhibition of Influenza Virus Production in Mice by Direct Delivery of Naked siRNA to the Respiratory System

This example confirms results above and demonstrates inhibition of influenza virus production in the lung by administration of siRNA targeted to NP to the respiratory system in an aqueous medium in the absence of delivery-enhancing agents. Six μg, 15 μg, 30 μg, and 60 μg of NP-1496 siRNAs or 60 μg of GFP-949 siRNAs in PBS were intranasally instilled into mice essentially as described above, except that mice were intranasally infected with PR8 virus, 1000 pfu per mouse, two hours after siRNA delivery. Lungs were harvested 24 hours after infection. NP-specific siRNA was effective for the inhibition of influenza virus when administered by intranasal instillation in an aqueous medium in the absence of delivery agents. A significant and dose-dependent difference in virus titer was seen between mice in each of the three treated groups and the controls (Table 16).

TABLE 16
Inhibition of Influenza Virus Production in the Lung Using
Naked siRNA

| Treatment | TCID50 | | | | | Average | P value |
|---|---|---|---|---|---|---|---|
| PBS | 125 | 365.5 | 213.7 | 365.5 | 125 | 239.95 | |
| GFP (60 μg) | 125 | 213.7 | 213.7 | 213.7 | 365.5 | 226.32 | |
| NP (6 μg) | 213.7 | 213.7 | 125 | 213.7 | 42.7 | 161.8 | 0.263 |
| NP (15 μg) | 125 | 125 | 42.7 | 25 | 73.1 | 78.17 | 0.024 |
| NP (30 μg) | 8.5 | 125 | 42.7 | 125 | 14.6 | 63.18 | 0.019 |
| NP (60 μg) | 73.1 | 14.6 | 25 | 25 | 25 | 32.54 | 0.006 |

### EXAMPLE 10

### Inhibition of Influenza Virus by Oraltracheal Delivery of Naked siRNA to the Respiratory System

siRNA preparation, viral infection, lung harvests, and influenza virus titer assays were performed as described above. Mice were anesthetized using avertin (administered by intraperitoneal injection). 1 mg/kg siRNA was delivered in a volume of 175 μl by oraltracheal injection.

siRNA (NP-1496), 1 mg/kg, and 30 μl Infasurf in 5% glucose was administered to groups of mice (5 mice per group). Mice were infected with influenza virus (2000 PFU) 3 hours after siRNA administration. Lungs were harvested 24 hours post-infection and virus titer measured.

In a second experiment, Black Swiss mice were anesthetized using intraperitoneally administered avertin. NP-1496 siRNA and GFP-949 siRNA in PBS was intratracheally administered into the mice, 50 μl each mouse. A third group that received PBS alone served as a control. Three hours later, the mice were anesthetized again using isofluorane/O₂, 30 μl of PR8 virus (2000 pfu=4× lethal dose) was intranasally administered into the mice. Twenty-four hours after infection, the mouse lungs were harvested, homogenized and virus titer was measured by evaluation of the TCID₅₀ as described above. Serial 5-fold dilutions of the lung homogenate were performed rather than 10-fold dilutions.

In summary, these results demonstrate the efficacy of siRNA delivered to the respiratory system in an aqueous medium in the absence of specific agents to enhance delivery.

### EXAMPLE 11

### Intranasal Delivery of siRNA Inhibits Influenza Production in Mice

The present example demonstrates that prophylactic intranasal administration of siRNA targeted to viral NP transcripts inhibited influenza virus replication and reduced viral RNA levels in a dose-dependent manner in the mouse.

Influenza normally infects and replicates in the upper respiratory tract and lungs. Therefore, due to accessibility, topical administration, i.e., intranasal and/or pulmonary delivery of drug should be ideal for influenza prophylaxis and therapy. Specifically, intranasal and/or pulmonary delivery of siRNAs is advantageous in treating influenza virus infection, because, (1) high local siRNA concentration are easily achieved when local delivery route is used and thus less siRNA is required compared to systemic delivery and (2) intranasal and/or pulmonary delivery methods are non-invasive. Thus, an intranasal delivery of siRNA in the influenza mouse model was pursued.

Intranasal administration of siRNA (unmodified, in PBS or saline) can be detected in the lungs and is able to silence endogenous gene expression or inhibit virus production in lung tissue. To test the efficacy of non-invasive delivery of influenza targeting siRNA, the NP-1496 siRNA (in PBS) was delivered intranasally. BALB/c mice were treated intranasally with indicated amounts of NP specific siRNA in PBS or PBS control. Two hours later, all mice were infected intranasally (1000 pfu/mouse) with the PR8 serotype. The lungs were harvested 24 hours post-infection and viral titer was measured from lung homogenates by MDCK-HA assay. P values between PBS and siRNA groups indicated statistical significance with 0.5, 1 and 2 mg/kg siRNA treated groups.

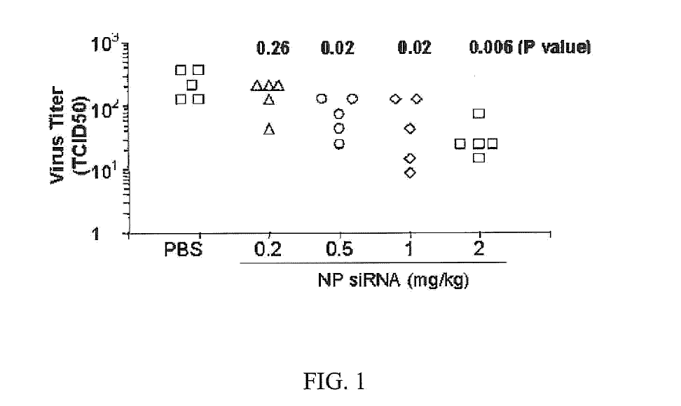

As shown in FIG. 1, in the absence of a carrier, naked NP targeting siRNA was effective in suppressing viral production in the mouse lung 24 hours post-infection. Suppression was dose dependent, with a 7-fold reduction being observed when 2 mg/kg of siRNA was delivered two hours prior to infection.

The effects of intranasal delivery of NP-targeting siRNA were also investigated at higher concentrations (10 mg/kg, delivered 3 hours prior to infection) using target mRNA expression (quantitative RT-PCR) and viral titer (MDCK-HA) to measure efficacy. BALB/c mice were administered control and NP-targeting siRNA intranasally (10 mg/kg, in PBS). Three hours later, all the mice were infected intranasally with PR8 virus (50 pfu/mouse). The lungs were harvested at 24 and 48 hours post-infection and total RNA was isolated from the left lung. Total mRNA was reverse transcribed to cDNA using dT18 primers (SEQ ID NO: 221). Real time PCR was carried out using PB1 specific primers to quantify viral mRNA levels. GAPDH was used as an internal control. The right and middle lungs were homogenized and the viral titer was measured by MDCK-HA assay.

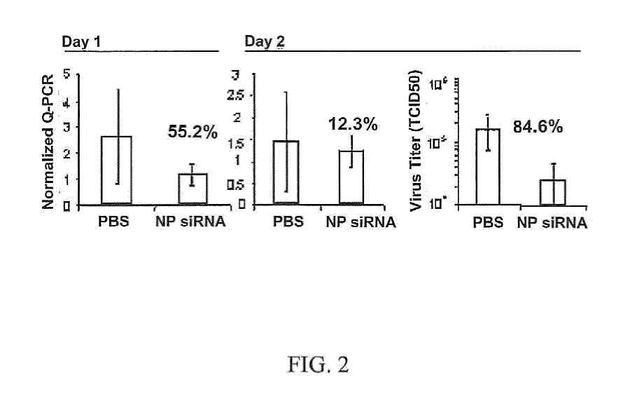

The results are shown in FIG. 2, which compares the normalized quantitative PCR results and the viral titer assay results. Viral mRNA level measured at 24 hours post-infection show a 55.2% inhibition, but by 48 hours post-infection only minimal inhibition was observed. In contrast, the MDCK-HA assay of mouse lung samples indicated 84.6% viral titer suppression on Day 2. Compared to the MDCK-HA assay that measures live virus particles, viral mRNA quantification is probably more sensitive in reflecting the early changes in viral replication. Thus, the decrease in viral mRNA suppression on Day 2 is probably due to the decreased RNAi effect in the mouse lung by that time.

Also, the effect on influenza viral titer in mouse between naked siRNA targeting the NP transcript delivered intranasally and the influenza treatment, an oseltamivir drug was compared. Relative to the level of viral titer observed with the GFP control siRNA, both the intranasally delivered naked siRNA and oseltamivir drug treatments reduced influenza viral titers.

The effect on viral titer by NP-viral transcript targeting in mouse after intranasal delivery the siRNA G1498 (INFsi-8) was also addressed. The G1498 siRNA exhibited significant ability to reduce viral titers in vitro and thus was chosen for further characterization in vivo. The control for this study was an unmodified siRNA targeted against luciferase (Dharmacon; Luc). Ten week old female BALB/c (Taconic) mice with a weight range of 18-22 grams were used in the study. There were ten mice per study group. The mice were dosed with G1498 siRNA in PBS at 2 mg/kg, 5 mg/kg, 10 mg/kg, 20 mg/kg and 30 mg/kg. The control groups were dosed the same, except no controls received a 2 mg/kg dose. Both the G1498 and Luc siRNA control groups were infected with PR8 influenza virus at 30 pfu in 30 μl in PBS four hours post-siRNA administration. Forty-eight hours post-infection, the mouse lungs were harvested and viral titers measured therefrom in MDCK cells with a TCID₅₀ assay.

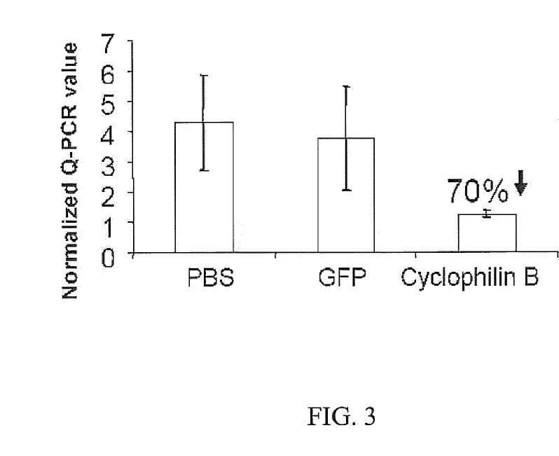

As shown in FIG. 3, the results of the TCID₅₀ assay indicate that the G1498 siRNA at 2 mg/kg suppressed influenza production in the mouse lung by 86%, at 5 mg/kg and 10 mg/kg by 90.6%, at 20 mg/kg by 96.6% and at 30 mg/kg by 95.2%. In relation to PBS alone or the Luc control siRNA study groups, the mice administered the G1498 siRNA intranasally, as a whole, showed significant differences (P<0.001). The mice that received PBS did not exhibit significant difference compared to the mice group that received the Luc siRNA, as a whole, (P>0.05). Each study group that received a dose of the G1498 siRNA significantly differed from the PBS group or Luc siRNA control study group at 30 mg/kg (P<0.05). Finally, no significant dose response was observed with mice that received the range of G1498 siRNA doses.

### EXAMPLE 12

### Use of dsRNA Therapeutics Against Drug Resistant Influenza

In this example, it is shown that dsRNA RNAi therapeutics are active against a drug resistant influenza strain. More specifically, the influenza strains were oseltamivir-resistant variant viruses. The dsRNA therapeutic is active against the drug resistant virus and therefore advantageously lacks cross-resistance due to oseltamivir.

Oseltamivir-resistant variant viruses were generated from A/WSN/33 (WSN) subtype H1N1 using the reverse genetics system. A mutation H274Y was introduced by site-directed mutagenesis into the NA gene. See Abed et al., Antiviral Therapy 2004; 9:577-581. The viral segments were then transfected into 293T cells followed by rescue and amplification in MDCK cells. They were not passaged under drug pressure.

The viruses were grown in the allantoic cavity of 10-day-old embryonated chicken eggs (Charles River Laboratories, Wilmington, Mass.) at 37° C. Virus titers were measured using plaque assays. For plaque assays, serial 10-fold dilutions of the virus samples were added onto a monolayer of Madin-Darby canine kidney (MDCK) cells in 1% semisolid agar. Two days after infection, plaques were visualized by staining with crystal violet. Vero and MDCK cells were obtained from American type culture collection (ATCC) and were grown in DMEM containing 10% heat-inactivated FCS, 2 mM L-glutamine, 100 units/ml penicillin, and 100 μg/ml streptomycin at 37° C. under a 5% CO2/95% air atmosphere.

For the viral infectivity assay, Vero cells were Seeded at 6.5×10⁴ cells/well the day before transfection in 500 μl 10% FBS/DMEM media per well. Samples of 100, 10, 1, 0.1, and 0.01 nM stock of each dsRNA were complexed with 1.0 μl (1 mg/mL stock) of LIPOFECTAMINE 2000 (Invitrogen) and incubated for 20 minutes at room temperature in 150 μl OPTIMEM (total volume) (Gibco). Vero cells were washed with OPTIMEM, and 150 μl of the transfection complex in OPTIMEM was then added to each well containing 150 μl of OPTIMEM media. Triplicate wells were tested for each condition. An additional control well with no transfection condition was prepared. Three hours post transfection, the media was removed. Each well was washed 1× with 200 μl 1×PBS containing 0.3% BSA/10 mM HEPES/PS. Cells in each well were infected with WSN strain of Influenza virus at a MOI 0.01 in 200 μl of infection media containing 0.3% BSA/10 mM Hepes/PS and 4 μg/ml trypsin. The plate was incubated for 1 hour at 37° C. Unadsorbed virus was washed off with the 200 μl of infection media and discarded. 400 μl DMEM containing 0.3% BSA/10 mM Hepes/PS and 4 μg/ml trypsin was added to each well and gently on the side of the well, since the cells start to detach following infection. The plate was incubated at 37° C., 5% CO2, for 48 hours. 50 μl supernatant from each well was tested in duplicate by TCID50 assays (Tissue-Culture Infective Dose 50, WHO protocol) in MDCK cells and titers were estimated using Spearman and Karber formula.

Oseltamivir-resistant strains were tested in a viral infectivity assay in Vero cells as described above. As shown in Table 17, the maximum reduction in viral titer ranged from about 20-fold to over 100-fold with dicer substrate dsRNAs. For this assay, gene knockdown efficiencies were ˜80-99% for the dicer substrate dsRNAs. Moreover, the dicer substrate dsRNAs were active against the oseltamivir-resistant strains even at concentrations as low as about 1 pM.

TABLE 17
Anti-viral Activity of Dicer Substrate
dsRNAs Against Oseltamivir-resistant Influenza

| Viral titer | | | | | | |
|---|---|---|---|---|---|---|
| (PFU/ml) (×10³) | Concentration (nM) | | | | | |
| dsRNA | 100 | 10 | 1 | 0.1 | 0.01 | 0.001 |
| DX3030 | 4.12 | 2.18 | 2.21 | 2.32 | 7.79 | 3.36 |
| DX3044 | 1.89 | 2.46 | 4.80 | 1.57 | 0.779 | 1.23 |
| DX4046 | 13.9 | 3.94 | 8.25 | 3.94 | 0.438 | 2.70 |
| DX3048 | 0.598 | 2.88 | 1.00 | 0.722 | 0.677 | 1.68 |
| DX3050 | 2.80 | 2.21 | 0.779 | 3.07 | 2.21 | 4.96 |
| G1498 | 33.6 | 15.2 | 12.2 | 12.2 | 7.22 | 2.54 |
| Virus only | 44.7 | — | — | — | — | — |

The structure of some dsRNAs are shown in Table 18.

TABLE 18

Double-stranded RNAs

| | |
|---|---|
| RNAi Agent | SEQUENCES |

| | |
|---|---|
| DX3030 | (SEQ ID NO: 236) |

| | | |
|---|---|---|
| Influenza | Sense | 5′-GGAUCUUAUUUCUUCGGAGACAAdTdG-3′ |

(SEQ ID NO: 237)

| | |
|---|---|
| Antisense | 5′-CAUUGUCUCCGAAGAAAUAAGAUCCUU-3′ |

| | |
|---|---|
| DX2816 | (SEQ ID NO: 238) |

| | | |
|---|---|---|
| Non-target | Sense | 5′-UUCUCCGAACGUGUCACGUdTdT-3′ |

| | |
|---|---|
| Qneg | (SEQ ID NO: 239) |

| | |
|---|---|
| Antisense | 5′-ACGUGACACGUUCGGAGAAdTdT-3′ |

| | |
|---|---|
| DX2940 | (SEQ ID NO: 240) |

| | | |
|---|---|---|
| LacZ | Sense | 5′-CUACACAAAUCAGCGAUUUdTdT-3′ |

(SEQ ID NO: 241)

| | |
|---|---|
| Antisense | 5′-AAAUCGCUGAUUUGUGUAGdTdC-3′ |

| | |
|---|---|
| DX2742 | (SEQ ID NO: 242) |

| | | |
|---|---|---|
| PPIB | Sense | 5′-GGAAAGACUGUUCCAAAAAUU-3′ |

| | |
|---|---|
| MoCypB | (SEQ ID NO: 243) |

| | |
|---|---|
| Antisense | 5′-UUUUUGGAACAGUCUUUCCUU-3′ |

| | |
|---|---|
| DX 2744 | (SEQ ID NO: 244) |

| | | |
|---|---|---|
| G1498 | Sense | 5′-GGAUCUUAUUUCUUCGGAGdTdT-3′ |

| | |
|---|---|
| influenza | (SEQ ID NO: 245) |

| | |
|---|---|
| Antisense | 5′-CUCCGAAGAAAUAAGAUCCdTdT-3′ |

| | |
|---|---|
| DX3044 | (SEQ ID NO: 246) |

| | | |
|---|---|---|
| Influenza | Sense | 5′-AGACAGCGACCAAAAGAAUUCGGdAdT-3′ |

(SEQ ID NO: 247)

| | |
|---|---|
| Antisense | 5′-AUCCGAAUUCUUUUGGUCGCUGUCUUU-3′ |

| | |
|---|---|
| DX 4046 | (SEQ ID NO: 248) |

| | | |
|---|---|---|
| Influenza | Sense | 5′-CGGGACrTCrTAGCArTACrTrTACrTGAdCdA-3′ |

| | |
|---|---|
| modified | (SEQ ID NO: 249) |

| | |
|---|---|
| Antisense | 5′-rTGrTCAGrTAAGrTArTGCrTAGAGrTCCCGUU-3′ |

| | |
|---|---|
| DX 3048 | (SEQ ID NO: 250) |

| | | |
|---|---|---|
| Influenza | Sense | 5′-GAUCUGUUCCACCAUUGAAGAACdTdC-3′ |

(SEQ ID NO: 251)

| | |
|---|---|
| Antisense | 5′-GAGUUCUUCAAUGGUGGAACAGAUCUU-3′ |

| | |
|---|---|
| DX 3050 | (SEQ ID NO: 252) |

| | | |
|---|---|---|
| Influenza | Sense | 5′-UUGAGGAGUGCCUGAUUAAUGAUdCdC-3′ |

(SEQ ID NO: 253)

| | |
|---|---|
| Antisense | 5′-GGAUCAUUAAUCAGGCACUCCUCAAUU-3′ |

| | |
|---|---|
| DX 3046 | (SEQ ID NO: 254) |

| | | |
|---|---|---|
| Influenza | Sense | 5′-AUGAAGAUCUGUUCCACCAUUGAdAdG-3′ |

(SEQ ID NO: 255)

| | |
|---|---|
| Antisense | 5′-CUUCAAUGGUGGAACAGAUCUUCAUUU-3′ |

### EXAMPLE 13

### Efficacy of RNAi Agents for Influenza

In Table 19, it is shown that RNAi agents are active against subclinical influenza strain Influenza A/Texas/91 in HeLa cells.

TABLE 19
Reduction of Average TCID50/ml titers of
Influenza/A/Texas/91 in HeLa cells by RNAi Agents

Concentration

| RNAi Agent | 100 nM | 10 nM | 1 nM | 0.1 nM |
|---|---|---|---|---|
| G1498 | 426 | 472 | 460 | 1980 |
| DX3030 | 544 | 685 | 544 | 2700 |
| DX3050 | 544 | 1110 | 1350 | 985 |
| DX3048 | 1490 | 480 | 626 | 1370 |
| DX3046 | 2540 | 326 | 886 | 3060 |
| DX3044 | 1980 | 1720 | 3120 | 1110 |
| DX3148 | | 1450 | | |
| Qneg | | 4810 | | |
| Virus Only | 22100 | | | |

The results in Table 19 show that RNAi agents reduced viral titer of subclinical influenza strain Influenza A/Texas/91 in HeLa cells by up to 50-fold relative to virus-only infection.

### EXAMPLE 14

### Efficacy of RNAi Agents Against Highly Pathogenic Influenza

To assess the ability of H5N1 virus to grow in Vero cells, cells were infected with H5N1 strain of influenza virus at an MOI 0.01 and virus in the supernatant was collected at 2, 12, 24, 36, 48 and 72 hours post-infection. H5N1 virus reached peak titers 5×10⁷ pfu/ml by 24 hours post-infection. H5N1 replicates faster than most of the commonly used laboratory strains of Influenza virus with half time to peak titers only in 6 hours post-infection.

As a control, H5N1 growth kinetics was compared to a H3N2 strain A/Wyoming/3/03 and the viral growth was followed up to 72 hours post-infection in the absence of trypsin. H5N1 continued to grow and reached high titers, unlike H3N2 strain which grew to relatively low titers in the absence of trypsin.

In vitro efficacy studies with influenza H5N1 (A/Vietnam strain) were performed in Vero cells using Lipofectamine 2000™ at an MOI of 0.01 and 24 hours post-infection. The RNAi agents tested were NP-specific dicer substrates DX3030 and DX3029, the PB2-specific dicer substrate DX3044, and the PB1-specific dicer substrate DX3046. Transfections were run in triplicate and the plaque assay was done in duplicate. As shown in Table 20, these RNAi agents exhibited activity against a highly pathogenic strain of Influenza H5N1, Influenza A/VietNam/1203/04, as determined by reduction of viral titers in Vero cells.

TABLE 20
Reduction of Viral Titers of Influenza
A/VietNam/1203/04 in Vero Cells With RNAi Agents

| RNAi | Gene | Viral titers | Fold | % KD relative | % KD relative |
|---|---|---|---|---|---|
| Agent | target | (pfu/ml) | change | to Virus ctrl | to Qneg |
| DX3029 | NP | 2.24E+05 | 23.2 | 95.7% | 88.6% |
| DX3030 | NP | 1.98E+05 | 26.2 | 96.2% | 90.1% |
| DX3044 | PB2 | 4.78E+05 | 10.9 | 90.8% | 79.1% |
| DX3046 | PB1 | 5.88E+05 | 8.8 | 88.7% | 70.7% |
| DX3048 | PB1 | 1.55E+06 | 3.4 | 70.2% | 28.3% |
| DX3050 | PA | 1.34E+06 | 3.9 | 74.2% | 37.4% |
| DX3148 | — | 1.75E+06 | 3.0 | 66.3% | 20.3% |
| Dicer | | | | | |
| Qneg | | | | | |
| G1498 | NP | 5.80E+05 | 9.0 | 72.2% | 74.9% |
| 21mer | — | 2.30E+06 | 2.3 | 55.8% | 0.0% |
| Qneg | | | | | |
| Untreated | — | 5.22E+06 | 0 | 0.0% | 0.0% |
| virus | | | | | |
| control | | | | | |

In vitro efficacy studies with influenza H5N1 (A/Vietnam strain) were also performed in Vero cells at various concentrations of these RNAi agents. In Table 21, it is shown that up to about 80-fold reduction of viral titers was observed against the highly pathogenic strain Influenza A/VietNam/1203/04.

TABLE 21
Fold-decrease of Viral Titers of
Influenza A/VietNam/1203/04 in Vero Cells

| | Concentration | | | | |
|---|---|---|---|---|---|
| RNAi Agent | 100 nm | 10 nm | 1 nm | 0.1 nM | 0.01 nM |
| G1498 | 7.38 | 6.76 | 7.83 | 5.48 | 2.78 |
| DX3029 | 79.73 | 76.29 | 49.72 | 14.94 | 22.73 |
| DX3030 | 61.89 | 20.16 | 19.67 | 11.64 | 17.18 |
| DX3044 | 4.84 | 5.98 | 4.44 | 4.04 | 3.51 |
| DX3046 | 5.55 | 4.05 | 4.47 | 3.60 | 3.13 |
| DX3050 | 3.06 | 3.50 | 2.35 | 2.60 | 2.90 |
| Qneg avg | 2.11 | | | | |

### EXAMPLE 15

### Efficacy Determined by of RNAi Agents Against Influenza H3N2

In vitro efficacy studies with influenza H3N2 (A/Wyoming strain) were performed in Vero cells at various concentrations for several RNAi agents. The MOI was 0.01 and supernatants were harvested at 48 h post-infection. In Table 22, it is shown that up to about 28-fold reduction of viral titers was observed against Influenza H3N2/A/Wyoming.

TABLE 22
Average TCID50 for RNAi Agents against Influenza A/Wyoming

| | Concentration | | | |
|---|---|---|---|---|
| RNAi Agent | 10 nM | 1 nM | 0.1 nM | 0.01 nM |
| G1498 | 2.40E+05 | 3.26E+05 | 5.80E+05 | 5.44E+05 |
| DX3030 | 2.80E+05 | 3.70E+05 | 3.98E+05 | 8.86E+05 |
| DX3044 | 2.47E+05 | 2.80E+05 | 3.16E+05 | 1.57E+05 |
| DX3046 | 1.74E+05 | 6.26E+04 | 1.26E+05 | 2.70E+05 |
| DX3048 | 3.16E+05 | 9.68E+05 | 1.11E+06 | 1.03E+06 |
| DX3050 | 6.58E+05 | 1.00E+06 | 6.58E+05 | 8.04E+05 |
| virus only | 1.75E+06 | | | |

### EXAMPLE 16

### Cytokine Response Profile of RNAi Agents Against Influenza

In vitro cytokine response profile studies were performed in peripheral blood mononuclear cells (PBMC) from ferret blood. PBMCs isolated from pooled whole blood of ferrets were treated with dicer substrates DX3030 or DX3050 (Qneg served as a negative control). Untreated PBMCs (PBS alone) served as a negative control while PBMCs treated with either lipopolysaccahride (LPS) or pokeweed mitogen (PWM) served as positive cytokine induced controls.

Cytokine levels of INF-α, IFN-γ, TNF-α, IL-2, IL-4, IL-6, IL-10, IL-12p40 were measured by SYBR Green based quantitative RT-PCR with PCR primers specific to each cytokine. Briefly, ferret PBMCs were isolated by Ficoll gradient from pooled blood and plated at 5×10⁵ cells per well on a 24-well cell culture plate in 1 mL growth media. Transfections were carried out in triplicate with RNAiMAX (0.25 μL/150 μL growth media) and 100 nM DX3030, DX3050, or Qneg. Cells were incubated with the transfection mixture for 22 hours at 37° C. Positive control PBMCs were treated for 24 hours with 15 μg of either LPS or PWM.

The LPS and PWM treated PBMCs had an elevated cytokine profile response compared to the untreated (PBS) negative control. In contrast, PBMC transfected with DX3030 or DX3050 showed minimal to no cytokine profile response (i.e., gene expression levels as measured by RT-PCR of the aforementioned cytokines was minimal to none) relative to the LPS and PWM treated PBMCs indicating influenza viral titer and/or gene knockdown is due to RNAi.

### EXAMPLE 17

### In Vivo Efficacy of RNAi Agents Against Influenza

The efficacy of RNAi agents to reduce influenza viral titers in ferrets following repeated dosing with RNAi agents either in PBS or a liposomal formulation was evaluated. Twenty-eight male ferrets were separated into 7 groups of 4 ferrets per group. The study design for each group is shown below in Table 23.

TABLE 23
Study Design for RNAi Agents against Influenza A/Wyoming

| | |
|---|---|
| | RNAi Agent Dose |
| Delivery | (per kg/day) |

| Group | RNAi Agent | Composition | (mg) | (nmol) |
|---|---|---|---|---|
| 1 | DX3030 | PBS | 10 | N/A |
| 2 | DX3050 | PBS | 10 | N/A |
| 3 | DX3030 | Liposomal | 0.86 | 51.8 |
| | | Formulation | | |
| 4 | DX3050 | Liposomal | 0.86 | 51.8 |
| | | Formulation | | |
| 5 | DX2816 | Liposomal | 0.69 | 51.8 |
| | (Qneg) | Formulation | | |
| 6 | Liposomal | Liposomal | N/A | N/A |
| | Formulation | Formulation | | |
| | Alone | | | |
| 7 | PBS | PBS | N/A | N/A |

All animals were challenged on Day 0 by intranasal administration of influenza virus (A/Panama/2007/99; H3N2 subtype) at 1.0×10⁶ EID₅₀. Animals were administered intranasally 1 mL of a delivery composition described in Table 23 on Day-2, Day-1, Day 0 (prior to challenge with virus), Day 1, and Day 2. Animals were observed beginning on Day-3 to end of study (Day 7) for activity, weight, temperature, sneezing, lethargy, anorexia, dyspnea, nasal and ocular discharge, diarrhea, neurological signs, and other abnormalities.

To measure viral load, nasal washes (NW) from all animals were collected on Day-3 (pre-wash), Day 0 (prior to dosing and infection), Day 1, Day 2, Day 3, Day 4, Day 5, and Day 7 post challenge. All samples were frozen at ≦−70° C. until titration in 9-11 day-old embryonated chicken eggs. Viral load analysis was performed from the nasal washes using MDCK based TCID₅₀ assay with incubation of nasal washes on MDCK cells for 48 h and performing TCID50-HA assays after 48 hours. The viral titer results are shown below in Table 24.

TABLE 24
Average TCID₅₀ for RNAi Agents Against Influenza A/Wyoming

| | RNAi | Delivery | Viral Titer (TCID₅₀/mL) | | | | |
|---|---|---|---|---|---|---|---|
| Group | Agent | Composition | Day 1 | Day 2 | Day 3 | Day 4 | Day 5 |
| 1 | DX3030 | PBS | 7.53E+02 | 6.58E+03 | 2.80E+03 | 4.68E+04 | 2.48E+04 |
| 2 | DX3050 | PBS | 8.43E+02 | 3.85E+03 | 2.30E+03 | 5.87E+03 | 6.10E+03 |
| 3 | DX3030 | Liposomal | 2.53E+02 | 1.37E+04 | 1.13E+04 | 4.79E+04 | 8.53E+03 |
| | | Formulation | | | | | |
| 4 | DX3050 | Liposomal | 6.78E+02 | 1.34E+04 | 4.72E+04 | 3.57E+03 | 1.02E+04 |
| | | Formulation | | | | | |
| 5 | DX2816 | Liposomal | 1.91E+04 | 3.12E+04 | 1.03E+02 | 1.37E+04 | 1.42E+04 |
| | (Qneg) | Formulation | | | | | |
| 6 | Liposomal | Liposomal | 3.43E+05 | 4.00E+04 | 1.13E+03 | 9.70E+03 | 7.64E+02 |
| | Formulation | Formulation | | | | | |
| | Alone | | | | | | |
| 7 | PBS | PBS | 6.26E+03 | 3.26E+05 | 7.57E+03 | 1.03E+03 | 3.26E+03 |

Unformulated RNAi agents DX3030 (Group 1) and DX3050 (Group 2) reduced viral load by 7-fold and 8-fold, respectively, at Day 1 relative to PBS alone (Group 7), and 50-fold and 85-fold, respectively, at Day 2 relative to PBS alone. Liposomal formulated DX3030 (Group 3) reduced viral titer by about 25-fold at Day 1 and Day 2 post-infection relative to PBS alone. Liposomal formulated DX3050 (Group 4) reduced viral titer by about 10-fold at Day 1 and 25-fold at Day 2 relative to PBS alone. Liposomal formulated DX2816 (Qneg; negative control RNAi agent) did not reduce viral load at Day 1, but reduced viral titer by about 10-fold at Day 2 post-infection relative to PBS alone.

Overall, the percent body weight loss of the treatment groups was about 3 to 4% compared to the untreated virus control that showed 8% body weight loss as a result of influenza infection. There was no direct correlation between the body weight loss and efficacy; however, the body weight decrease of the treated groups was compared to the body weight decrease for the untreated virus control. Body temperature of the treated groups were found to be higher (e.g., 104-105° F.) especially at Day 2 post-infection compared to the virus control, vehicle alone and the liposomal formulated Qneg.

The cytokine expression profile of ferrets treated with DX3030, DX3050 and Qneg in PBS or a liposomal formulation was measured. The cytokine profile of ferret nasal washes collected at Day-3 and Day 0 were compared to the cytokine profile post-dosing. A panel of cytokines, including those reflecting early innate immune response activation were profiled, including tumor necrosis factor alpha (TNF-α), Interferon alpha (IFN-α), and interleukin-6 (IL-6), and to those indicating Th1 polarization, including IFN-γ, IL-2 and ILp12p40, and the Th2 cytokines IL-4 and IL-10. Since antibody reagents for ferrets are not available, published PCR primer sequences were used for semi-quantitative real-time RT-PCR (Svitek and Messling, 2007).

The early innate immune responses cytokines TNF-α, IFN-α and IL-6 were not upregulated at Day-3 and Day 0 (administration of RNAi agents began on Day-2) relative to PBS alone. Further, the cellular immune cytokine response, which indicates a Th1 polarization were also not upregulated as shown by similar levels of IFN-γ, IL-2 levels at Day-3 and Day 0 compared to PBS alone. IL12p40 expression levels were not upregulated compared to the PBS alone control. The cellular immune responses indicating a Th2 type of immune induction (IL-4 and IL-10) was not upregulated at Day-3 or Day 0, compared to PBS alone, after the administration of RNAi agent to ferrets.

What is claimed is:

1. A method for preventing or treating an influenza infection in a subject caused by a drug resistant strain of influenza comprising administering to the subject a therapeutically-effective amount of one or more RNAi-inducing agents having efficacy against the drug resistant strain.

2. The method of claim 1, wherein the drug resistant strain is resistant to an anti-viral drug.

3. The method of claim 2, wherein the anti-viral drug is a neuramidase inhibitor.

4. The method of claim 3, wherein the neuramidase inhibitor is selected from the group consisting of oseltamivir, zanamivir, and peramivir.

5. The method of claim 2, wherein the anti-viral drug is an M2 inhibitor.

6. The method of claim 5, wherein the M2 inhibitor is amantadine or rimantadine.

7. The method of claim 2, wherein the anti-viral drug is a amantadine or ribavarin.

8. The method of claim 1, wherein the one or more RNAi-inducing agents are selected from the group consisting of DX3030, DX3044, DX4046, DX3048, DX3050, and peptide conjugates thereof.

9. The method of claim 1, wherein the one or more RNAi-inducing agents are administered by intranasal delivery to a subject.

10. The method of claim 9, wherein the one or more RNAi-inducing agents are administered at a dose of from about 0.001 mg/kg to about 2 mg/kg.

11. The method of claim 9, wherein the one or more RNAi-inducing agents are administered at a dose of from about 0.006 mg/kg to about 0.6 mg/kg.

12. The method of claim 1, wherein the one or more RNAi-inducing agents are administered by pulmonary delivery to a subject.

13. The method of claim 12, wherein the one or more RNAi-inducing agents are administered at a dose of from about 0.001 mg/kg to about 5 mg/kg.

14. The method of claim 12, wherein the one or more RNAi-inducing agents are administered at a dose of from about 1 mg/kg to about 4 mg/kg.

15. A method for preventing or treating an influenza infection in a subject in need thereof comprising administering to the subject a therapeutically-effective amount of one or more RNAi-inducing agents in combination with a neuramidase inhibitor.

16. The method of claim 15, wherein the one or more RNAi-inducing agents and the neuramidase inhibitor are administered in series.

17. The method of claim 15, wherein the neuramidase inhibitor is administered to the subject within 24 hours of the administration of the one or more RNAi-inducing agents.

18. The method of claim 15, wherein the one or more RNAi-inducing agents are selected from the group consisting of DX3030, DX3044, DX4046, DX3048, DX3050, and peptide conjugates thereof.

19. A method for preventing or treating an influenza infection in a subject in need thereof comprising administering to the subject a therapeutically-effective amount of one or more RNAi-inducing agents having therapeutic efficacy against at least 90% of influenza viruses.

20. The method of claim 19, wherein the one or more RNAi-inducing agents have therapeutic efficacy against influenza A, influenza B, and highly pathogenic influenza viruses.

21. The method of claim 19, wherein the one or more RNAi-inducing agents have efficacy against H1N1, H3N2, and H5N1.

22. The method of claim 19, wherein the one or more RNAi-inducing agents are selected from the group consisting of DX3030, DX3044, DX4046, DX3048, DX3050, and peptide conjugates thereof.

23. A method for preventing or treating an influenza infection in a subject in need thereof comprising administering to the subject a therapeutically-effective amount of one or more RNAi-inducing agents which delay the emergence of a resistant influenza strain that is resistant to an RNAi-inducing agent, which is different from the one or more RNA-inducing agents.

24. The method of claim 23, wherein the one or more RNAi-inducing agents delay the emergence of the influenza strain by at least one or more passages in vitro.

25. The method of claim 23, wherein the one or more RNAi-inducing agents delay the emergence of the influenza strain by at least two or more passages in vitro.

26. The method of claim 23, wherein the one or more RNAi-inducing agents are selected from the group consisting of DX3030, DX3044, DX4046, DX3048, DX3050, and peptide conjugates thereof.

27. A method for preventing or treating an influenza infection in a subject in need thereof comprising administering to the subject a therapeutically-effective amount of two or more RNAi-inducing agents, wherein the two or more RNAi-inducing agents are targeted to different portions of the influenza genome and are administered in series.

28. The method of claim 27, wherein the two or more RNAi-inducing agents are targeted to a portion of an NP influenza gene or a PA influenza gene.

29. The method of claim 27, wherein the two or more RNAi-inducing agents are targeted to different portions of an NP influenza gene or a PA influenza gene.

30. The method of claim 27, wherein the two or more RNAi-inducing agents are targeted to a portion of an NP influenza gene and at least one of the RNAi-inducing agents is targeted to a portion of a PA influenza gene or a PB1 influenza gene.

31. The method of claim 27, wherein the two or more RNAi-inducing agents are selected from the group consisting of DX3030, DX3044, DX4046, DX3048, DX3050, and peptide conjugates thereof.

32. A method for preventing or treating an influenza infection in a subject in need thereof comprising administering to the subject a therapeutically-effective amount of one or more RNAi-inducing agents in combination with a neuramidase inhibitor.

33. The method of claim 32, wherein the one or more RNAi-inducing agents and the neuramidase inhibitor are administered in series.

34. The method of claim 32, wherein the neuramidase inhibitor is administered to the subject within 24 hours of the administration of the one or more RNAi-inducing agents.

35. The method of claim 32, wherein the amount of the neuramidase inhibitor drug administered to the subject is less than that amount that would have been indicated for treating or preventing the influenza infection in the subject by use of the neuramidase inhibitor drug alone in the absence of the one or more RNAi-inducing agents.

36. The method of claim 32, wherein the one or more RNAi-inducing agents are selected from the group consisting of DX3030, DX3044, DX4046, DX3048, DX3050, and peptide conjugates thereof.
